(19) United States
(12) Patent Application Publication
(10) Pub. No.: US 2024/0188886 A1
(43) Pub. Date: Jun. 13, 2024

(54) SYSTEM, METHOD AND APPLICATION TO CONVERT TRANSDERMAL ALCOHOL CONCENTRATION TO BLOOD OR BREATH ALCOHOL CONCENTRATION

(71) Applicant: UNIVERSITY OF SOUTHERN CALIFORNIA, Los Angeles, CA (US)

(72) Inventors: Gary Rosen, Los Angeles, CA (US); Susan Luczak, Los Angeles, CA (US); Chunming Wang, Los Angeles, CA (US); Jay Bartroff, Los Angeles, CA (US); Larry Goldstein, Los Angeles, CA (US)

(73) Assignee: UNIVERSITY OF SOUTHERN CALIFORNIA, Los Angeles, CA (US)

(21) Appl. No.: 18/275,108

(22) PCT Filed: Feb. 4, 2022
(86) PCT No.: PCT/US2022/015371

§ 371 Date: Jul. 31, 2023

Related U.S. Application Data
(60) Provisional application No. 63/146,299, filed on Feb. 5, 2021.

(51) Int. Cl. A61B 5/00; G16H 40/67; G16H 50/30

(52) U.S. Cl. CPC A61B 5/4845; A61B 5/6801; A61B 5/7264; G16H 40/67; G16H 50/30

(57) ABSTRACT

System, method and application that obtains, consolidates, and integrates multiple sources of data including Transdermal Alcohol Concentration (TAC) along with drinking diary, photo/video, breath analyzer, other biological data (e.g., heart rate, skin conductance. blood flow, person-level biometrics), and environmental data (e.g., ambient temperature, humidity, GPS) and uses models described herein to convert TAC obtained from a wearable biosensor into estimated Blood Alcohol Concentration (BAC) or Breath Alcohol Concentration (BrAC).

### CROSS-REFERENCE TO RELATED APPLICATIONS

This application is based upon and claims priority to U.S. provisional patent application 63/146,299 entitled “SYSTEM, METHOD AND APPLICATION TO CONVERT TRANSDERMAL ALCOHOL CONCENTRATION TO BLOOD OR BREATH ALCOHOL CONCENTRATION” and filed on Feb. 5, 2021, the entire content of which is incorporated herein by reference.

### STATEMENT AS TO FEDERALLY SPONSORED RESEARCH

This invention was made with government support under contract numbers R01-AA-026368 and R21-AA-017711 awarded by the National Institutes of Health (NIH). The government has certain rights in this invention.

### BACKGROUND

### 1. Field

This disclosure relates generally to measuring blood alcohol concentration, and more specifically, to measuring transdermal alcohol concentration (TAC) and calculating blood or breath alcohol concentration.

### 2. Description of the Related Art

Alcohol concentration is frequently measured via blood or breath tests that evaluate the blood alcohol concentration (BAC) or breath alcohol concentration (BrAC) respectively. However, such tests require a high degree of cooperation from a test subject, interrupt a test subject's ongoing activities, and must be administered by a trained person under certain conditions to provide accurate results. Efforts to develop a wearable sensor that provides for continuous monitoring, monitoring without interrupting the test subject's ongoing activity, or more convenient monitoring include measurement of transdermal alcohol concentration (TAC). However, TAC is not readily converted to BAC or BrAC and a relationship between TAC and BAC or BrAC may change based on various factors. Thus, there is a need for a system, method, and device for monitoring TAC and reliably converting TAC to BAC or BrAC.

### SUMMARY

This invention develops and provides a software/mobile application (app) that obtains, consolidates, and integrates multiple sources of data including Transdermal Alcohol Concentration (TAC) along with drinking diary, photo/video, breathalyzer (BrAC), other biological data (e.g., heart rate, skin conductance, blood flow, person-level biometrics), and environmental data (e.g., ambient temperature, humidity, GPS) and uses models developed to convert TAC obtained from a wearable biosensor into estimated Blood Alcohol Concentration (BAC) or Breath Alcohol Concentration (BrAC). Sensor(s) and/or biosensor(s) are used to measure the biological data and the environmental data, and the processor(s) is/are used to combine or process this data and produce the estimated BAC and/or BrAC. The one or more drinking curves from a population of humans, the biological data, the environmental data, the static characteristics, and the physiological characteristics may be stored in a memory for use by the processor. The invention utilizes processors, computers, computer programs and/or software to incorporate the data via models and algorithms to produce the estimated BAC and/or BrAC.

As mentioned, a method is provided. The method is for converting transdermal alcohol concentration (TAC) to blood or breath alcohol concentration (BAC/BrAC). The method may include measuring, using a biosensor, the TAC of a human. The method may include receiving, by a processor, data corresponding to one or more drinking curves for a population of humans. The method may also include receiving, by the processor, data corresponding to at least one of (i) static characteristics of the human, (ii) physiological characteristics of the human, and (iii) current environmental conditions. Finally, the method may include, converting, using the processor, the TAC to BAC/BrAC using the data from one or more drinking carves, and the at least one of (i) the static characteristics of the human, (i) the physiological characteristics of the human, and (iii) the current environmental conditions.

In various embodiments, the data corresponding to the one or more drinking curves includes a measurement of TAC and a measurement of at least one of BAC and BrAC. The data corresponding to the one or more drinking curves may include a time sequence of measurements of TAC and a time sequence of measurements of BAC or BrAC and may be performed in real time. The data corresponding to the static characteristics may include a measurement of at least one of age, sex, ethnicity, height, weight, body fat and muscle, skin color, skin thickness, and skin tortuosity. The data corresponding to the physiological characteristics may include a measurement of at least one of sweat, skin conductance, skin hydration, exercise, heart rate, blood pressure, blood flow, and stomach content. The data corresponding to the current environmental conditions may include a measurement of at least one of ambient temperature, humidity, pressure, GPS, weather, and climate. The converting may be performed using a deterministic or stochastic finite dimensional autoregressive moving average with exogenous input (ARMAX) input/output model. The converting may be performed using a blind or Bayesian deconvolution scheme. The converting may be performed using a lattice filter-based recursive identification scheme. The converting may be performed using an artificial neural network (ANN) by the processor, wherein the processor is remote from the biosensor and connected to the biosensor by a network. The converting may be performed using a physics-informed neural network (PNN) The network may be a wireless connection to the internet. The converting may be performed using a deconvolution filter based on output feedback linear quadratic Gaussian tracking gain computed by the processor. The converting may be performed using first principles physics-based forward model(s) with random parameters having distributions fit to population BrAC/TAC data. The fitting the distributions may be based on a naïve pooled or mixed effects statistical model using either maximum likelihood, method of moments, or Bayesian techniques. The converting may be performed in real-time with progressive forecasting and modeling techniques and recursive updating methods.

A system for converting transdermal alcohol concentration (TAC) to blood or breath alcohol concentration (BAC/BrAC) may be provided. The converting may be in real-time. The converting may be with progressive forecasting and modeling techniques and recursive updating methods. The system may include a biosensor for measuring the TAC of a human. The system may include a processor. The processor may be configured to receive data from one or more drinking curves from a population of humans. The processor may be configured to receive data corresponding to at least one of (i) static characteristics of the human. (ii) physiological characteristics of the human, and (iii) the current environmental conditions. The processor may be configured to convert in real-time the TAC to BAC/BrAC using the data from one or more drinking curves and the at least one of (i) the static characteristics of the human, (ii) the physiological characteristics of the human, and (iii) the current environmental conditions. In various embodiments, the processor is remote from the biosensor and is connected to the biosensor via a network.

In various embodiments, the system includes a remote database containing the one or more drinking curves from the population of humans connected to the processor via a network. The system may include a plurality of further biosensors connected to the processor via a network, wherein the processor coverts, in real-time the TAC to BAC/BrAC for each of the plurality of further biosensors. The data corresponding to the one or more drinking curves may include a measurement of TAC and a measurement of at least one of BAC and BrAC. The data corresponding to the static characteristics may include a measurement of at least one of age, sex, ethnicity, height, weight, body fat and muscle, skin color, thickness, and tortuosity The data corresponding to the physiological characteristics may include a measurement of at least one of sweat, skin conductance, skin hydration, exercise, heart rate, blood pressure, blood flow, and stomach content. The data corresponding to the current environmental conditions may include a measurement of at least one of ambient temperature, humidity, pressure, GPS location data, weather, and climate.

The converting may be performed using a deterministic or stochastic finite dimensional autoregressive moving average with exogenous input (ARMAX) input/output model. The converting may be performed using a blind or Bayesian deconvolution scheme. The converting may be performed using a lattice filter-based recursive identification scheme. The converting may be performed using an artificial neural network (ANN) by the processor, wherein the processor is remote from the biosensor and connected to the biosensor by a network. The converting may be performed using a physics-informed neural network (PNN) The network may be a wireless connection to the internet. The converting may be performed using a deconvolution filter based on output feedback linear quadratic Gaussian tracking gain computed by the processor. The converting may be performed using first principles physics-based forward model(s) with random parameters having distributions fit to population BrAC/TAC data. The fitting the distributions may be based on a naïve pooled or mixed effects statistical model using either maximum likelihood, method of moments, or Bayesian techniques. The converting may be performed in real-time with progressive forecasting and modeling techniques and recursive updating methods.

In various embodiments, a biosensor device is provided. The device may be for converting transdermal alcohol concentration (TAC) to blood or breath alcohol concentration (BAC/BrAC). The device may include a wearable sensor contactable to a human skin to measure the TAC of the human. The device may include a processor connected to the wearable sensor and connectable to a network. The processor may be configured to receive, via the network, data corresponding to one or more drinking curves for a population of humans. The processor may be configured to convert TAC to BAC/BrAC using (i) the data from one or more drinking curves and (ii) the measured TAC.

### BRIEF DESCRIPTION OF THE DRAWINGS

Other systems, methods, features, and advantages of the present invention will be or will become apparent to one of ordinary skill in the art upon examination of the following figures and detailed description.

FIG. 1A depicts a system for converting transdermal alcohol concentration (TAC) to blood or breath alcohol concentration (BAC/BrAC), in accordance with various embodiments;

FIG. 1B depicts a system for converting multiple TACs to BAC/BrAC for multiple biosensors, in accordance with various embodiments;

FIG. 1C depicts a method for converting transdermal alcohol concentration (TAC) to blood or breath alcohol concentration (BAC/BrAC), in accordance with various embodiments;

FIG. 2 depicts values of the \hat{q} estimators calculated from the simulated data for 20 observations, in accordance with various embodiments;

FIG. 3 depicts values of the \hat{q} estimators calculated from the simulated data for 60 observations, in accordance with various embodiments;

FIG. 4 depicts values of the \hat{q} estimators calculated from the simulated data for 100 observations, in accordance with various embodiments;

FIG. 5 depicts a range and distribution of BrAC observations, in accordance with various embodiments;

FIG. 6 depicts a range and distribution of TAC observations, in accordance with various embodiments;

FIG. 7 illustrates a chart 700 of BrAC, TAC observations and estimated BrAC that results from using the minimizer \hat{q}=(0.6341,0.7826), in accordance with various embodiments;

FIG. 8A shows the values for the loss functions over a number of iterations, in accordance with various embodiments;

FIG. 8B shows the of q₁ and q₂ over the number of iterations over the number of iterations, in accordance with various embodiments;

FIG. 9A shows a distribution for 88 selected drinking episodes using a standard normal distribution for the prior of the latent variable, in accordance with various embodiments;

FIG. 9B shows a distribution using the posterior over the latent variable, in accordance with various embodiments;

FIG. 9C displays the distribution for the full set of 126 drinking episodes with a standard normal prior for the latent variable, in accordance with various embodiments;

FIG. 9D shows the corresponding distribution using the posterior distribution for the latent variable, in accordance with various embodiments;

FIG. 10A shows a distribution of the posterior latent variable with a historgram for 88 drinking sessions used as training data, in accordance with various embodiments;

FIG. 10B shows the histogram for 126 drinking sessions used as training data, in accordance with various embodiments;

FIGS. 11A-D show results for four selected drinking episodes using the parameter distribution from training the GAN with all 126 drinking episodes, in accordance with various embodiments;

FIGS. 12A-F show a comparison of predicted BAC/BrAC signals using different drinking episodes, in accordance with various embodiments;

FIG. 13 depicts functional control gains, in accordance with various embodiments;

FIG. 14 depicts observer gains, in accordance with various embodiments; and

FIG. 15 depicts a chart with a shaded region is the 90% credible band centered at the mean for the optimal functional control gains ƒ₃ computed using the disclosed method, in accordance with various embodiments.

### DETAILED DESCRIPTION

A system, method and/or a mobile application (app) that converts Transdermal Alcohol Concentration (TAC) to estimated Blood or Breath Alcohol Concentration (BAC/BrAC) in real-time and post-drinking, by using a novel collection of data from biosensors, self-report, and the environment.

With the goal of well-founded statistical inference on an individual's blood alcohol level based on noisy measurements of their skin alcohol content, this disclosure develops M-estimation methodology in a general setting. Discussions herein then apply it to a diffusion equation-based model for the blood/skin alcohol relationship thereby establishing existence, consistency, and asymptotic normality of the nonlinear least squares estimator of the diffusion model's parameter and the resulting estimated blood alcohol curve. Simulation studies show agreement between the performance of these estimators and their asymptotic distributions, and the results are applied to a real skin alcohol data set collected via biosensor.

A goal is to model and estimate a human subject's alcohol concentration in the blood (BAC) or breath (BrAC) as a function of the alcohol level measured at the skin, i.e., the transdermal alcohol concentration (TAC), via a biosensor. Approximately 1% of the alcohol ingested in the human body is metabolized through the skin. For decades it has been recognized that the levels of TAC are connected to those of BAC/BrAC, but also that there are challenges in modeling this relationship. Because alcohol has to pass from the blood through the skin to be captured by a TAC sensor placed on the surface of the skin, it is subject to variation across individuals (e.g., skin layer thickness, porosity, tortuosity, etc.) and drinking episodes (e.g., ambient temperature, humidity, subject activity level, skin hydration, vasodilation, etc.). These effects result in a TAC-BAC/BrAC relationship that can be highly variable. Thus, TAC devices to date have typically been primarily used only in legal and research settings as abstinence monitors (e.g., in court mandated monitoring of DUI offenders) because of difficulties researchers have found translating raw TAC to the quantity of alcohol in the blood.

Still, TAC measured by a wearable biosensor device has great potential as a tool to improve personal and public health. It provides a passive, unobtrusive way to collect naturalistic data for extended periods of time. The same is not true about BrAC, which typically must be measured by trained research staff in the laboratory under controlled conditions using a breath analyzer, and thus is less practical for capturing alcohol levels in the field under real-world conditions. Moreover, the breath analyzer requires a user to be compliant, potentially interferes with naturalistic drinking patterns, and is subject to inaccuracy (e.g., readings too high due to mouth alcohol, or too low due to not properly taking a deep lung breath for a reading). Thus, creating a system that reliably converts TAC data into estimates of BAC (or BrAC) would greatly benefit the alcohol research and clinical communities who, along with public health institutes, have been quite interested in such models. Such a tool would dramatically improve the accuracy of field data and the validity of naturalistic studies of alcohol-related health outcomes, disease progression, treatment efficacy, and recovery. A wearable alcohol monitoring device could have consumer appeal as well, helping individuals monitor their own alcohol levels and make better health choices.

Work on the TAC-BAC/BrAC relationship begins with deterministic models for the “forward process” of the propagation of alcohol from the blood, through the skin, and its measurement by the sensor. Other approaches reverse the forward process to estimate BrAC based on the TAC. These efforts show unaccounted for variation in the TAC-BAC/BrAC relationship and subsequent work began to incorporate uncertainty into the models via a random diffusion equation. Other statistical modeling approaches include a regression model for peak BrAC using peak TAC, time of peak TAC, and gender using controlled laboratory data. Other efforts examine time delays from peak BrAC to peak TAC. Further efforts use physics-based statistical models for the TAC-BAC/BrAC relationship.

In this disclosure, systems, methods, and devices are presented to meet this challenge by using a physics-based statistical model that allows individual, device, and drinking episode level variation by treating the data from each person/device/episode triple as resulting from its own model parameters. Discussions herein determine the large sample behavior of estimates of these parameters and give conditions under which these estimates are consistent and have a limiting normal distribution. These discussions then use those results to give a statistically rigorous asymptotic characterization of the properties of the BrAC/BAC curve estimates obtained from measured TAC, including information on estimation error. As these estimates are made on an individualized basis, they will not be adversely affected when used in a study of a population whose characteristics vary widely. On the other hand, these estimates—in some embodiments—require individualized calibration over subject, device and environmental conditions

While the discussion includes calibration aspects, in further embodiments, the key model parameters depend on measurable subject and environmental covariates which may be measured, and which eliminates some or all calibration.

It may, in some embodiments be desirable to quantitatively estimate BAC/BrAC from TAC to within the desired degree of accuracy after first calibrating the underlying models to each individual subject and situation, thus accounting for confounding person-level, environmental, and physiological factors that differ across the population of subjects and across situations. The forward and inversion models included in the app are sophisticated mathematical systems that include deterministic and population models and supervised learning algorithms.

First, the forward model captures the dynamics of the transport of ethanol molecules from the blood through the skin and its measurement(s) by the biosensor. The app includes the option to calibrate the forward model based on individualized data obtained from a real-time drink diary, retrospective drink diary, or pre-set drinking paradigm, or based on population-based models alone or combined with individualized personal data (e.g., age, sex, ethnicity, skin, height, weight, body fat, etc.)

Then, in the inversion process the model is used to deconvolve estimated BAC/BrAC in future drinking episodes from measured TAC and all other available data. This means the BAC/BrAC in subsequent drinking episodes is estimated from the TAC provided by the biosensor without any further action by the user.

The real-time deconvolution scheme to estimate BAC/BrAC uses novel models that incorporate adaptive real-time data driven model refinement/learning, autoregressive moving average with exogenous input (ARMAX), and lattice filter-based recursive identification schemes to produce estimates in real-time, and which can be continuously updated with new data. An additional approach to recovering BAC/BrAC from TAC includes a real-time deconvolution scheme based on a technique from linear control and estimation theory. Farther mechanisms are also discussed.

Once drinking is complete for an episode and TAC has returned to or established a baseline, the app uses the full set of data to update the model BAC/BrAC estimates using the entire set of data for the episode. In addition, over time individuals can update their personalized model fits with data obtained through the app and paired biosensors in additional drinking sessions. As data accumulates for an individual subject, Bayesian techniques are used to improve the accuracy of the estimated BAC/BrAC. Finally, the app also includes components for capturing subjective responses to alcohol (e.g., feeling flushed, intoxicated) and drinking context (e.g., vis photos, video, GPS location) beyond alcohol consumption, using automated reminders, random prompts, and/or self-timed diary entries options, and this data can then be paired to estimated BAC/BrAC and other biosensor measurements.

The output includes TAC and estimated BAC/BrAC curves with credible bands, additional biosensor data and subjective ratings of alcohol response displayed alone and in conjunction with estimated BAC/BrAC, and summary scores of drinking events along with correlations with subjective ratings of alcohol response and drinking contexts. Summary scores will also be retained and displayed in a calendar format, which will also allow for retrospective recording of drinking sessions when not wearing a TAC biosensor. This multi-faceted app provides comprehensive assessment and result options for capturing drinking in real-time and consolidating this data into meaningful metrics. This multifaceted app provides a comprehensive system that incorporates all available data, utilizes self-report through a novel web application, and includes real-time forecasting of estimated BAC/BrAC curves and scores. This app is the first effective tool for non-experts to produce quantitative estimates of BAC/BrAC from TAC and other data.

A wearable biosensor (e.g., a digital watch, fuel cell, Fitbit®) may be used to measure or sense ethanol molecules from the blood via the skin. The system is based on a fit forward model in the form of a partial differential (diffusion) equation that captures the dynamics of the transport of ethanol molecules from the blood through the skin and its measurement by the biosensor. The system then uses the estimated model to deconvolve estimated BAC/BrAC from the biosensor measured TAC. The accuracy of the estimated BAC/BrAC is significantly improved by correcting for environmental and physiological factors that differ across the population of subjects and situations. Therefore, it is important that the underlying models be, in some form, calibrated to each subject, device, and situation The system utilizes sophisticated mathematical population models and supervised learning algorithms together with the capability to optionally enter drinking diary, breathalyzer, and other biosensor data to tune the underlying models to the physiological characteristics of the person wearing the device and the current environmental conditions. The BAC/BrAC for all drinking episodes can then be estimated from the TAC passively provided by the biosensor without any active participation by the user.

This invention extends the scope of application of TAC to BAC/BrAC conversion software and adjusts for variations (i) between subjects, (ii) within subjects, (iii) in environmental conditions, (iv) across hardware devices, and/or (v) in repeated measurements over time, when applying the diffusion model, and (vi) can be fit in real-time. In particular, the invention utilizes statistical models for the low dimensional input parameters to the diffusion equations that depend on covariate information that describe characteristics of the subjects and their environment. The end result is personalized, real-time BAC/BrAC estimates with accompanying statistical accuracy measures, such as credible intervals and margins of error. In addition, the invention provides a theoretical, asymptotic analysis of the performance of the new estimation methods that result upon embedding the models in the underlying diffusion equation.

The invention utilizes adaptive real-time data driven model refinement/learning For example, the invention has the ability to incorporate real-time drink diary data into one or more of the underlying physics-based models described earlier to construct an adaptive/recursive data assimilation, estimation, and prediction system. The models are continuously updated with newly available real-time individual-level data to produce revised/estimated BAC/BrAC based on TAC in real-time. Even though the underlying state equation that forms the basis of this invention is, in general, infinite dimensional, end-to-end, it is a single input/single output linear time invariant system. Thus, BAC/BrAC can be approximated using a deterministic or stochastic finite dimensional autoregressive moving average with exogenous input (ARMAX) input/output model. The invention further includes lattice filter-based recursive identification schemes, which allow for the efficient modification of both the order of the model and the parameters when new data is introduced into the system. The invention takes advantage of the wealth of real-time adaptive parameter estimation, filtering, prediction, and deconvolution schemes available for systems described by these types of models. The invention accounts for the introduction of nonlinearities into these schemes through the use of artificial neural networks (ANNs) and trains them using a variant of back propagation. This scheme yields a somewhat delayed estimated BAC/BrAC, which can then be augmented by a prediction scheme to yield preliminary real-time estimated BAC/BrAC, and afterwards update estimated BAC/BrAC for the entire episode.

The invention incorporates new innovations that serve to improve the efficiency and accuracy of the estimated BAC/BrAC. In particular, two approaches to deconvolving the BAC/BrAC signal from the TAC signal have been included. One approach used to recover BAC/BrAC from TAC is based directly on our physiological model for the diffusion of ethanol through the epidermal layer of the skin. While this approach provides an effective low rank parameterization of the relationship between BAC/BrAC and TAC when there was extremely limited experimental data, a more empirical model can offer more flexibility when a relatively rich pool of laboratory collected contemporaneous matched BAC/BrAC-TAC data is available.

In an empirical linear model, we assume that the measured TAC is the convolution of two random signals, the convolution kernel K(s; ω) and the measurement error θ(s; ω). That is,

y_{TAC}(t; ω)=∫_{-∞}^{t}K(t−s; ω)v_{BrAC}(s)ds+θ(s; ω).

The process of training the model given in the above equation consists of identify reliable distributions for the functions K and θ based on available matched BAC/BrAC-TAC pairs. Since both functions belong to an infinite-dimensional space of random functions, effective parameterization of these function spaces is crucial to ensure stability of the training process. Inspired by the physiological model, a family of cubic spline functions defined on a strategically selected non-uniform grid is chosen. Analysis of the optimally determined kernel functions from a set of BAC/BrAC-TAC pairs exhibited an encouraging level of consistency among test subjects and data from different sessions for the same test subject.

Using the resulting distributions for K and θ obtained through analysis of data for an appropriate cohort or population, the retrieval of BAC/BrAC from TAC is done in bear real-time by calculating statistically consistent and efficient estimators for BAC/BrAC. One example of such an estimator is given by

$${\overline{v}}_{BrAC} = arg {min}_{{v}_{BrAC}} ( {\sum }_{k = 1}^{n} {\vert {\overline{y}}_{TAC} ( {t}_{k} ) - {\int }_{- ∞}^{{t}_{k}} K ( {t}_{k} - s ; ω ) {v}_{BrAC} ( s ) ds \vert}^{2} + {‖ K ( \cdot ; ω ) - \overline{K} ( \cdot ) ‖}^{2} ) ,$$

where y̅_{TAC}(tₖ) represents the measured TAC value at time tₖ and K̅ corresponds to the population mean for the kernel functions. Note that in the optimization above, the calculation obtains an optimal pair of estimators. v̅_{BrAC} and K(⋅; ω) As data accumulates for an individual subject, Bayesian techniques are used to improve the accuracy of the retrieved BAC/BrAC signal.

Another approach to recovering BAC/BrAC from TAC includes a real-time deconvolution scheme based on a linear control and estimation theory technique. By formulating the deconvolution problem as a linear quadratic Gaussian tracking problem, the estimated BAC/BrAC signal is obtained in the form of a linear output feedback law. More precisely, the estimated BAC/BrAC signal is given as a real-time linear function of the measured TAC signal. Undesirable non-physical oscillations in the estimates which result from the underlying ill-posedness of the filtering problem being solved to determine the BAC/BrAC signal are mitigated by including an appropriate penalty term in the quadratic performance index. This approach also yields credible bands and error bars along with the estimated BAC/BrAC signal.

Beyond the mathematical models, this software invention includes real-time and retrospective self-report data collection mobile app for recording drinking diary, breathalyzer, other biosensor data, drinking context, and other factors that vary over a drinking episode (e.g., stomach contents, mood, behavior). The app includes the option to add calibration data from individualized data obtained from a real-time drink diary, retrospective drink diary, pre-set drinking paradigm, or based on population-based models combined with individualized personal data (e.g., age, sex, ethnicity, skin, height, weight, body fat, etc.). The app also includes components for capturing subjective responses to alcohol (e.g., feeling flushed, intoxicated) and drinking context (e.g., via photos, video, GPS location) beyond alcohol consumption, using automated reminders, random prompts, and/or self-timed diary entries options, and these data can then be paired to estimated BAC/BrAC and other biosensor measurements. Summary scores of drinking events along with correlations with subjective ratings of alcohol response and drinking contexts will be calculated and displayed in episode-level figures and charts These summary scores will also be retained and displayed for multiple drinking episodes in a calendar format, which also will allow for retrospective recording of drinking sessions.

The invention is implemented using a combination of hardware and software. The hardware includes the TAC biosensor, processors, memories, displays, and environmental sensors. The software includes computer code that can run on the hardware. The invention allows the user the option to select which method(s) they would like to use through a set of menus, based on what the user prioritizes to optimize, similar to factor analyses options in commercially available statistical software where the user can select different matrix rotations or fit indices to emphasize in the model runs. The invention produces both estimated BAC/BrAC curves, credible bands, and summary scores such as maximum estimated BAC/BrAC, time of maximum BAC/BrAC and area under the BAC/BrAC curve.

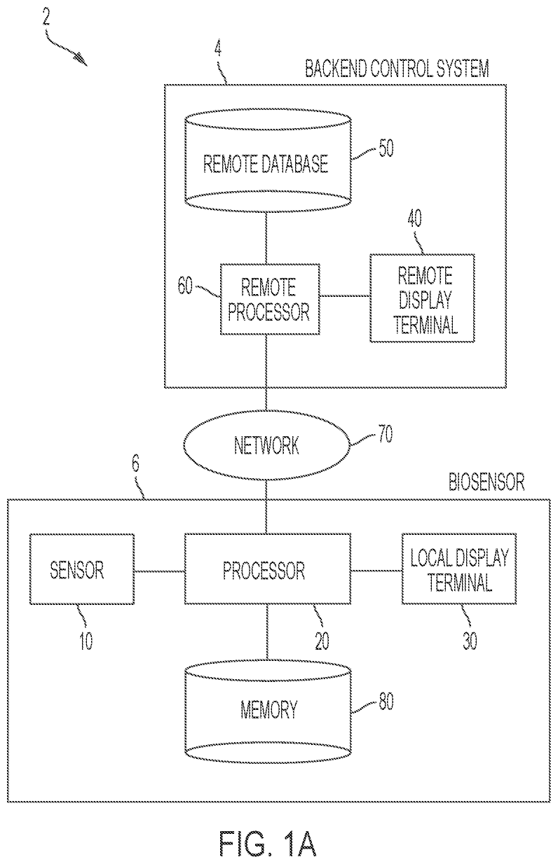

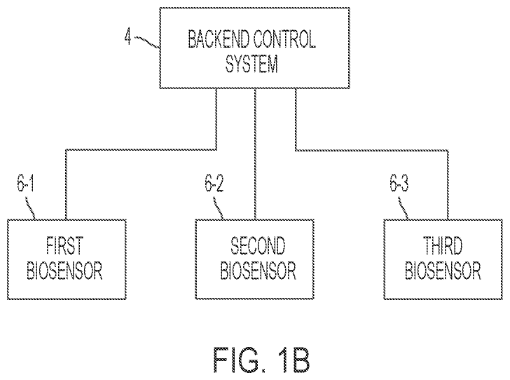

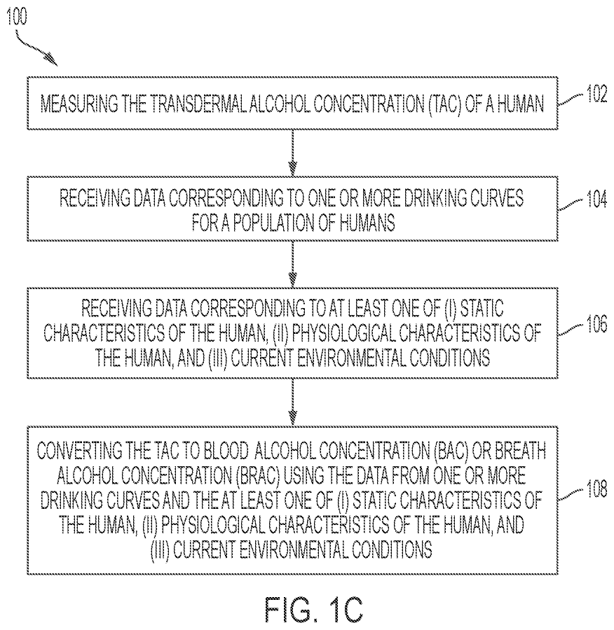

With reference to FIG. 1A, system 2 for converting transdermal alcohol concentration (TAC) to blood or breath alcohol concentration (BAC/BrAC) in real-time may include a biosensor 6 connected to a backend control system 4. In various embodiments, the biosensor is a wearable device. In further instances, the biosensor is a combination of devices interconnected by a body area network. For instance, aspects of the biosensor may be worn adjacent a user's skin and other aspects of the biosensor may be carried in a pocket, a purse, or otherwise near or about the person of a user. The biosensor 6 measures a biological indicator, such as ethanol present in sweat or on/in a user's skin. The biosensor 6 may provide data representative of the biological indicator to a backend control system 4 for processing and may receive in return, an indication of the user's BAC/BrAC. In further instances, the biosensor 6 is not connected to a backend control system 4 and instead performs calculations on a local processor to determine an indication of the user's BAC/BrAC. In various embodiments, the biosensor 6 is selectably connectable to the backend control system 4. For instance, the biosensor 6 may perform calculations locally when disconnected from the backend control system 4 or may provide data to the backend control system 4 for the performance of calculations by the backend control system when connected to the back end control system 4. In further embodiments, the biosensor 6 stores data representative of the biological indicator when disconnected from the backend control system 4 and provides this data to the backend control system 4 when a connection is established. In this manner, the biosensor 6 may measure a TAC and the biosensor 6 and/or a backend control system 4 may calculate a corresponding BAC/BrAC. In various embodiments, the biosensor 6 and/or the backend control system 4 may display the corresponding BAC/BrAC in human readable form, such as on a display terminal.

The biosensor 6 may include a sensor 10. The sensor 10 may include an element configured to measure a TAC. For instance, a fuel cell device may process ethanol present on a user's skin or in a user's sweat to generate electricity, which may be measured. Because the voltage, current, power, and/or other measurable aspect of the generated electricity may be quantified, the corresponding amount of ethanol responsible for generating the electricity may be quantified. Various references to sensor 10 elsewhere herein provide example sensors for various embodiments.

The biosensor 6 may include a processor 20. The processor may be a computer, of a microcontroller, or a low power embedded microprocessor, or a single-board computer, an application-specific integrated circuit (ASIC) or any other electronic data processing device as desired. In various embodiments, the processor 20 is connected to a memory 80. The memory 80 may be a working memory, providing for data storage during calculation by the processor 20 of BAC/BrAC from TAC. The memory 80 may be a storage memory, such as for storage of data corresponding to TAC prior to transmission of this data to a backend control system 4. The memory maybe both a storage memory and a working memory.

The biosensor 6 may have a local display terminal connected to the processor 20. The local display terminal 30 may be a human-readable interface. For instance, the local display terminal 30 may be one or more LED, audio annunciator, tactile feedback device, LCD or other text or graphic display, or any other apparatus configured to provide information in human-readable form. In various embodiments, the local display terminal provides menu structures and other interface elements of an application as described herein. In various embodiments, the local display terminal displays a TAC measurement. In further embodiments, the local display terminal displays a calculated BAC/BrAC measurement calculated by the biosensor 6, the backend control system 4, or a combination of the biosensor 6 and the backend control system 4 that is calculated from a measured TAC.

The biosensor 6 may be connectable to a network 70. The backend control system 4 may also be connectable to the network 70. The network 70 may permit electronic communication between the biosensor 6 and the network 70. In various embodiments, the network 70 comprises the internet. In further embodiments, the network 70 may be a private network, or a virtual private network, or an RF data link, or an optical data link, or a wired link, or any electronic connection. The network 70 may include wireless aspects, such as cellular connections, or Wi-Fi connections or other aspects.

Having discussed the biosensor 6 and a network 70, attention is now directed to a backend control system 4. The backend control system 4 may comprise a server, or a cloud computing resource, or any other computing system as desired. In various instances, the backend control system 4 provides greater processing power than the biosensor 6 and facilitates calculation of BAC/BrAC from TAC by remotely handling calculations and other processing tasks. In various instances, the backend control system 4 collects and aggregates data from the biosensor 6 with data from other resources, such as user inputs, stored or laboratory research data, previously collected data such as prior TAC data, user-specific data such as weight, height, and other aspects, training data, and/or the like. In various instances, the backend control system 4 collects and aggregates data from multiple different biosensors 6. Various data, factors, and relevant variables are discussed throughout, each of which may be processed. stored, or otherwise received by the backend control system 4 and/or the biosensor 6.

The backend control system 4 may include a remote database 50. The remote database 50 may store the aforementioned data, TAC calculations, BAC/BrAC calculations and/or the like. The remote database 50 may provide both working memory and/or storage memory.

The backend control system may include a remote processor connected to the remote database 50 and the network 70. The remote processor may a computer, or a microcontroller, or a low power embedded microprocessor, or a single-board computer, an application-specific integrated circuit (ASIC) or any other electronic data processing device as desired. The remote processor may be a distributed or cloud computing resource.

The backend control system 4 may have a remote display terminal 40 connected to the remote processor 60. The remote display terminal 40 may be a human-readable interface. For instance, the remote display terminal 40 may be one or more LED, audio annunciator. tactile feedback device, LCD or other text or graphic display, or any other apparatus configured to provide information in human-readable form. In various embodiments, the remote display terminal 40 provides menu structures and other interface elements of an application as described herein. In various embodiments, the remote display terminal 40 displays a TAC measurement. In further embodiments, the remote display terminal 40 displays a calculated BAC/BrAC measurement calculated by the biosensor 6, the backend control system 4, or a combination of the biosensor 6 and the backend control system 4 that is calculated from a measured TAC. The remote display terminal 40 may be separate from the backend control system 4 and connected to the network 70. The remote display terminal 40 may be browser session of a user accessing the backend control system 4, such as via a website login interface on an internet browser running on a commodity personal computer.

Previously, it was mentioned that the backend control system 4 may collect and aggregate data from multiple different biosensors 6. In addition, the backend control system 4 may provide processing resources to multiple different biosensors for calculating a BAC/BrAC from a measured TAC. With reference to FIG. 1B, in various instances, a backend control system 4 is connected to a first biosensor 6-1, a second biosensor 6-2, and a third biosensor 6-3. The backend control system 4 may be connected to any number of biosensors. While not illustrated in FIG. 1B, in various embodiments, the biosensors and the backend control system 4 may be connected via a network 70.

Thus, in various instances, the system 2 for converting transdermal alcohol concentration (TAC) to blood or breath alcohol concentration (BAC/BrAC) in real-time may include a biosensor 6 for measuring the TAC of a human. The system may include a processor. The processor may be a processor 20, a remote processor 60, or a combination of the processor 20 and remote processor 60 such that certain processes are conducted on processor 20 and other processes are conducted on remote processor 60. As such, one or more of the processors may receive data from one or more drinking curves from a population of humans. One or more of the processors may receive data corresponding to at least one of (i) static characteristics of the human, (ii) physiological characteristics of the human, and (iii) the current environmental conditions. One or more of the processors may convert in real-time the TAC to BAC/BrAC using the data from one or more drinking curves and the at least one of (i) the static characteristics of the human, (ii) the physiological characteristics of the human, and (iii) the current environmental conditions.

Moreover, the biosensor 6 for converting transdermal alcohol concentration (TAC) to blood or breath alcohol concentration (BAC/BrAC) may include a wearable sensor 10 contactable to a human skin to measure the TAC of the human and a processor (processor 20, remote processor 60, and/or a combination of processor 20 and processor 60) connected to the wearable sensor 10 and connectable to a network 70, the processor configured to receive, via the network 70, data corresponding to one or more drinking curves for a population of humans. One or more of the processor may be configured to convert TAC to BAC/BrAC using (i) the data from one or more drinking curves and (ii) the measured TAC.

Turning now to FIG. 1C, a method 100 of calculating a BAC/BrAC from TAC may be provided. One may appreciate that the various calculations discussed elsewhere herein may be implemented by such a method 100 and such a method 100 may be implemented by the embodiments of FIG. 1A-B. A method 100 for converting transdermal alcohol concentration (TAC) to blood or breath alcohol concentration (BAC/BrAC) may include multiple steps. For instance, the method may include measuring, using a biosensor, the TAC of a human (block 102). The method may include receiving by a processor data corresponding to one or more drinking curves for a population of humans (block 104). Notably, the processor may be a processor local to the biosensor 6 (FIG. 1A, processor 20) or may be a remote processor (FIG. 1A, remote processor 60).

The method may include receiving, by a processor, data corresponding to at least one of (i) static characteristics of the human, (ii) physiological characteristics of the human, and (iii) current environmental conditions (block 104). Again, the processor may be a processor local to the biosensor 6 (FIG. 1A, processor 20) or may be a remote processor (FIG. 1A, remote processor 60). This processor may be a different processor than that referred to in block 104.

Finally the method may include converting, using a processor, the TAC to BAC/BrAC using the data from one or more drinking curves, and the at least one of (i) the static characteristics of the human, (ii) the physiological characteristics of the human, and (iii) the current environmental conditions (block 106). This processor may be the processor 20 (FIG. 1A) or remote processor 60 (FIG. 1B) and may be a same or different processor as that of blocks 104 and/or 106.

Various methods for such converting are discussed at length throughout this disclosure. For instance, the converting may be performed using a deterministic or stochastic finite dimensional autoregressive moving average with exogenous input (ARMAX) input/output model. The converting may be performed using a blind or Bayesian deconvolution scheme. The converting may be performed using a lattice filter-based recursive identification scheme. The converting may be performed using an artificial neural network (ANN). The converting may be performed using a hidden Markov model (HMM) or a physics-informed bidden Markov model (PIHMM). The converting may be performed using a deconvolution filter based on output feedback linear quadratic gaussian tracking gain. Moreover, the converting may be performed using first principle physics-based forward models with random parameters having distributions fit to population BrAC/TAC data. The fitting the distributions may be based on a naive pooled or mixed effects statistical model using either maximum likelihood, method of moments, or Bayesian techniques. The converting may be performed in many different ways. The converting may be performed in real-time with progressive forecasting and modeling techniques and recursive updating.

Thus, one may appreciate that the method may have various additional aspects. For instance, the data corresponding to the one or more drinking curves may be different types of data. The data may be a measurement of TAC. The data may be a measurement of BAC. The data may be a measurement of BrAC. The data may include comparisons of TAC to BAC and/or BrAC.

The data that corresponds to the static characteristics may include a variety of different measurements. For instance, the measurements may relate to aspects of a specific human for whom TAC is being measured. The measurements may include a measurement of at least one of age, sex, ethnicity, height, weight, body fat and muscle, skin color, skin thickness, and skin tortuosity.

The data that corresponds to the one or more physiological characteristics may include a variety of different measurements. For example, the measurements may relate to aspects of a specific human for whom TAC is being measured but which may be dynamic. For instance, the measurements may include a measurement of at least one of sweat, skin conductance, skin hydration, exercise, heart rate, blood pressure, blood flow, and stomach content.

The data that corresponds to the current environmental conditions may include a variety of different measurements. For example, the measurements may relate to aspects of an environment that the human for whom TAC is being measured is exposed to. The measurements may include a measurement of at least one of ambient temperature, humidity, pressure, GPS, weather, and climate.

Having provided an overview of the system, method, and device above, attention is now directed to a discussion of a diffusion model to characterize ethanol diffusion across skin so that correspondingly the subject's BAC/BrAC may be model as a function of TAC.

The following discussion will include various types of models. For instance, a partial differential equation diffusion model may characterize alcohol transfusion across the skin. A least squares approach may be provided for estimating an unknown vector. M-estimation is provided and basic examples of its use, as well as an application of M-estimation to the mentioned model. Yet further, the application of the M-estimation to the partial differential equation diffusion model may be implemented to obtain results on the performance of resulting BrAC curves estimated from TAC. The discussion will also include an evaluation of theoretical results in simulations and an illustration using BrAC/TAC relationships measured experimentally.

Diffusion Model (Section 1). Although a goal is to model a human subject's BAC/BrAC as a function of TAC, the ethanol molecules themselves move in the other direction: from the blood, through the skin, to ultimately be measured by the sensor on the surface of the skin. Thus the relevant physics describe the TAC as a function of BAC/BrAC. Consider a specific model for this transport based on Fick's law of diffusion which depends on an unknown, 2-dimensional parameter q=(q₁, q₂). The result is TAC expressed as a convolution of BAC/BrAC with a kernel or filter, and as a function of the unknown q which may then be estimated via nonlinear least squares as described and whose properties are considered in Section 3. These properties determine the inferential consequences for BAC/BrAC estimation, and in particular have a large impact on the accuracy of the estimated BrAC curve, as studied in Section 3.

Let x(t, η) denote the concentration of ethanol at time t≥0 and depth η∈ [0,1] from the skin surface through epidermis, choosing units so that μ(t)=x(t, 1), t≥0 is the BAC at time t. A Fick's law-based model has been developed and used successfully to model data of this type. The model specifies x(t, η) as the solution to the partial differential equation, with boundary condition

$$\frac{\partial x}{\partial t} = {q}_{1} \frac{{\partial }^{2} x}{\partial {η}^{2}} , {q}_{1} \frac{\partial x}{\partial η} {\vert}_{η = 1} = {q}_{2} μ ( t ) , {q}_{1} \frac{\partial x}{\partial η} {\vert}_{η = 0} = x {\vert}_{η = 0} , \qquad ( 1 )$$

depending on the parameter q=(q₁, q₂). The TAC at skin level is then x(t, 0). When we want to emphasize dependency on the parameter q we will write, for instance, μ(t; q).

The system with its boundary conditions can be solved in continuous time in terms of unbounded linear operators, with solution

x(t)=e^{A(q)t}x(0)+∫₀^{t}e^{A(q)(t-s)}B(q)μ(s)ds. (2)

In cases we consider, x(0) will be the zero function, that is, observation begins at, or before, the time of first intake of alcohol. By taking a discretization of the distance η from skin level into k steps for some k sufficiently large, the operators in (2) can be approximated by k dimensional linear operators (i.e., matrices) yielding the approximation to the solution given by

x^{(k)}(t)=∫₀^{t}e^{A(k)}^{_{(q)(t−s)}}B^{(k)}(q)μ(s)ds. (3)

Now fixing, and suppressing in the notation, the level of discretization k, an observation taken at time t can be represented as the linear function of x(t) given by

ds, (4)

plus an additive error term. For observations taken at skin level, the vector C will have a one in its first component, and zeros elsewhere.

The matrices in (4) depend on the unknown parameter q as

A(q)=q₁D+E and B(q)=q₂F, (5)

where C, D, E, and F are known matrices that result from making the finite-dimensional approximation discussed. More precise assumptions and properties of these matrices, and the domain of q, will be specified in Section 3.

Non-Linear Least Squares Estimation (Section 1.2). To estimate the parameter q, we assume that TAC data {yᵢⱼ, 1≤i≤n, 1≤j≤mᵢ} is collected on a single individual over n different drinking episodes at the my times 0≤t_{i,1}< . . . <t_{i,m}_{ᵢ}≤Tᵢ, for given BrAC curves μᵢ on [0, Tᵢ]. With m=(m₁, . . . , mₙ), the estimator minimizes

$${J}_{n , m} ( q ) = \frac{1}{2 {\sum }_{i = 1}^{n} {m}_{i}} {\sum }_{i = 1}^{n} {\sum }_{j = 1}^{{m}_{i}} {( {f}_{{μ}_{i}} ( {t}_{ij} ; q ) - {y}_{ij} )}^{2} , \qquad ( 6 )$$

where ƒ_{μ}_{ᵢ}(tᵢⱼ; q) is given by the right hand side of (4) with μ replaced by μᵢ, the BrAC curve for drinking episode i. The model specified by (4) and (5) is deterministic, but to account for measurement variability, we include additive, homoscedastic errors on the observed values TAC values. The constant variance condition implies that all TAC observations are ‘equally reliable’, and that the error variances, in particular, do not depend on the length of time elapsed since the last observation. For that reason, the least squares objective functions give equal weight to their summands, and when appropriate, weights, inversely proportional to variance, could be included. We may also allow the length of the time interval Tᵢ of the i^{th} episode, and the location of the sampling times, to be stochastic.

In Section 2 below, we consider the existence, consistency, and limiting distribution of our least squares estimators in a general M-estimation context, and present some examples. In Section 3 we apply the results in Section 2 to the diffusion model of Section 1, and present Theorems 3.1 and 3.3, which contain our main results on inference for the main parameter q of interest, and also for the error variance σ². In Section 4 we apply the results of Section 3 for making inference on the BrAC curve, and in particular for the construction of uniform error bounds on the resulting curve estimate. We validate our theoretical work via simulation and real data analysis in Section 5.

M-Estimation (Section 2)—Existence, Consistency, and Limiting Distribution. In this section we consider M-estimation in a general setting that contains what we will require to handle the diffusion model we consider. Prior discussions of M-estimation tend to focus on the case of a univariate parameter, whereas ours covers the multivariate case. Prior efforts cover only least squares estimation whereas our results apply to the more general estimating equation (7). Also, previous results only apply to approximate normality and require i.i.d. error terms, whereas our Theorem 2.2 can be applied to other limiting distributions and relaxed conditions on the error terms, although our main application is to limiting normality. Finally, previous results are more restrictive in terms of a number of technical conditions, such as compactness of the parameter space Θ which our results do not require, and the existence of “tail products” of vectors of observation means and error terms, which our results eschew in favor of more conventional regularity conditions on the score type function Uₙ.

After establishing the notation and setup in Section 2.1, we state our main results in Section 2.2. In Section 2.3 we provide some general examples of the applications of our results to least squares and maximum likelihood estimation.

Set Up and Summary of Results (Section 2.1). For n≥1, observed data X⁽ⁿ⁾ in a space χ⁽ⁿ⁾, a parameter space Θ⊂ [symbol] ^{p} having non-empty interior, and a function Uₙ:Θ×χ⁽ⁿ⁾→ [symbol] ^{p}, consider the estimating equation

Uₙ(θ)=0, θ∈Θ, (7)

where the dependence of [symbol] ₙ on the data is suppressed. In our examples χ⁽ⁿ⁾ will a Euclidean space endowed with a family of densities pₙ(x⁽ⁿ⁾; θ), θ∈Θ which generate the data from this family with θ=θ₀. Two important situations in which the solutions of such equations arise are maximum likelihood and least squares estimation.

For maximum likelihood, under smoothness conditions on the densities, the maximizer of the log likelihood Lₙ(θ)=log pₙ(x⁽ⁿ⁾; θ) is given as a solution to (7) with

[symbol]

ₙ(θ)=∂_{θ}Lₙ(θ; X⁽ⁿ⁾), (8)

where ∂_{θ} denotes taking derivative with respect to θ, resulting in a column vector of partial derivatives when θ itself is a vector. When the data X⁽ⁿ⁾ consists of n independent random vectors X₁, . . . , Xₙ in [symbol] ^{d}, each with distribution p(x; θ₀), the space χ⁽ⁿ⁾ can be identified with [symbol] ^{d×n}, and pₙ(x⁽ⁿ⁾, θ) is the product of the marginal densities p(xᵢθ) for i=1, . . . , n.

To introduce least squares estimation, suppose that pairs (xᵢ, yᵢ)∈ [symbol] ^{d}× [symbol] , i=1, . . . , n, are observed with distribution depending on θ for which [symbol] _{θ}[yᵢ|xᵢ]=ƒᵢ(xᵢ; θ) for ƒᵢ(x; θ) in some parametric class of functions. With x⁽ⁿ⁾=(x₁, . . . , xₙ), the least squares estimate of θ is given as the minimizer of

$$J ( θ ; {x}^{( n )} ) = \frac{1}{2 n} {\sum }_{i = 1}^{n} {( {y}_{i} - {f}_{i} ( {x}_{i} ; θ ) )}^{2} ,$$

which under smoothness conditions can be obtained via (7) with

$${𝒰}_{n} ( θ ) = {\partial }_{θ} J ( θ ; {x}^{( n )} ) = \frac{1}{n} {\sum }_{i = 1}^{n} ( {f}_{i} ( {x}_{i} ; θ ) - {y}_{i} ) {\partial }_{θ} {f}_{i} ( {x}_{i} ; θ ) , \qquad ( 9 )$$

The aim of the estimating equation Uₙ(θ)=0 is to provide a value close to the one where the function Uₙ(θ) takes the value of 0 in some expected, or asymptotic, sense. In particular, in Theorem 2.1 we will show, under that when Uₙ(θ₀) is, under an appropriate scaling, close to zero as n→∞, then the sequence of estimates obtained via the estimating equations will be consistent for the true parameter.

In Theorem 2.2, we will also provide a corresponding limiting distribution for solutions to the estimating equation (7). Let Uₙ(, θ) have components

Uₙ(θ)=(U_{n,j}(θ))_{1≤j≤p} where U_{n,j}: [symbol] ⁿ×Θ→ [symbol] .

In the case of maximum likelihood estimation, where we have (8), under the assumption of the existence and continuity of second derivatives of Lₙ for θ∈Θ, writing U_{n′}/(θ) as short for the observed information matrix ∂_{θ}Uₙ^{T}(θ)∈ [symbol] ^{p×p}, its k, j^{th} component is given by

$$\frac{\partial {U}_{n , j} ( θ )}{\partial {θ}_{k}} = \frac{{\partial }^{2} {L}_{n} ( θ )}{\partial {θ}_{k} \partial {θ}_{j}} = \frac{{\partial }^{2} {L}_{n} ( θ )}{\partial {θ}_{j} \partial {θ}_{k}} = \frac{\partial {U}_{n , k} ( θ )}{\partial {θ}_{j}} .$$

And in this case, the third condition in (11) below is equivalent to the condition that the limiting information matrix l is positive definite. Tolerating a slight abuse of notation, we may also write ∂ⱼ rather than ∂_{θ}_{ⱼ} when taking a partial with respect to the j^{th} coordinate variable, and ∂ⱼ^{m} for the m^{th} order derivative, for instance, denoting the k, j^{th} entry of U_{n′}(θ) by ∂ₖU_{n,j}(θ).

Over each coordinate j=1, . . . , p, under second order differentiabilty conditions, we will make use of the second order Taylor expansion of U_{n,j}(θ) around some θ₀∈Θ,

$${U}_{n , j} ( θ ) = {U}_{n , j} ( {θ}_{0} ) + {\sum }_{k = 1}^{p} {\partial }_{k} {U}_{n , j} ( {θ}_{0} ) ( {θ}_{k} - {θ}_{k , 0} ) + \frac{1}{2} {\sum }_{1 \leq k , l \leq p} ( {θ}_{k} - {θ}_{k , 0} ) {\partial }_{k , l} {U}_{n , j} ( {θ}_{n , j}^{*} ) ( {θ}_{l} - {θ}_{l , 0} ) , \qquad ( 10 )$$

where each 0*_{n,j} lies on the line segment connecting θ and θ₀. In the following, we let ∥⋅∥ denote the Euclidean norm of a vector, the operator norm of a matrix, and the supremum norm of a function.

Estimating equations, consistency, and asymptotic normality (Section 2.2). We now present results that provide conditions for the consistency and existence of a non-trivial limiting distribution for a properly centered and scaled sequence of estimating equation solutions. We also include results on the consistent estimation of parameters on which the asymptotic distribution of our estimate may depend.

Theorem 2.1 Suppose that Uₙ:Θ×χ⁽ⁿ⁾→ [symbol] ^{p} is twice continuously differentiable in an open set Θ₀⊂Θ containing θ₀, and that there exist a sequence of real members aₙ, a matrix Γ∈ [symbol] ^{p×p} and γ>0 such that

$${a}_{n} {U}_{n} ( {θ}_{0} ) {\to }_{p} 0 and {a}_{n} {U}_{{n}^{′}} ( {θ}_{0} ) {\to }_{p} Γ as \qquad ( 11 )$$

$$n \to ∞ with {inf}_{‖ θ ‖ = 1} {θ}^{T} Γθ = γ .$$

Suppose further that for any η∈(0,1), that there exists a K such that for all n sufficiently large,

P(|aₙ∂_{k,l}U_{n,j}(θ)|≤K, 1≤k,l,j≤p, θ∈Θ₀)≥1−η. (12)

Then for any given ⊂>0 and η⊂(0,1), for all n sufficiently large, with probability at least 1−η there exists \hat{θ}ₙ∈Θ satisfying Uₙ(\hat{θ}ₙ=0 and ∥\hat{θ}ₙ−θ₀∥≤ϵ, that is, a sequence of roots to the estimating equation (7) consistent for θ₀.

In addition, for any sequence \hat{θ}ₙ→ₚθ₀, we have

aₙU_{n′}(\hat{θ}ₙ)→_{pΓ,} (13)

that is, Γ can be consistently estimated by aₙU_{n′}(\hat{θ}ₙ) from any sequence consistent for θ₀.

Proof: By replacing Uₙ by aₙUₙ and θ by θ−θ₀, we may assume that the conditions of Theorem 2.1 hold with aₙ=1 and θ₀=0. For δ>0 let

B_{δ}={θ:∥θ∥≤δ}.

For the given η∈(0,1), let K and n₀ be such that (12) holds with η replaced by η/2 for n≥n₀. For the given ϵ>0, take δ∈(0, ϵ) such that B_{δ}⊂Θ₀ and Cδ<γ where

$$C = 2 + \frac{{Kp}^{3 / 2}}{2} .$$

By (11) there exists n₁≥n₀ such that for n≥n₁

P(∥Uₙ(0)∥<δ²)≥1−η/3 P(∥U_{n′}(0)−Γ<δ)≥1−η3, (14)

and also taken large enough so that (12) holds with η replaced by η/3. By the union bound, all three events hold with probability at least 1−η. For θ∈B_{δ} and θ*_{n,j} given by (10), the components of Rₙ(θ)=(R_{n,1}(θ), . . . , R_{n,p}(θ))^{T} as defined by

$${R}_{n , j} ( θ ) = {\sum }_{1 \leq k , l \leq p} {θ}_{k} {\partial }_{k , l} {U}_{n , j} ( {θ}_{n , j}^{*} ) {θ}_{l} satisfy \vert {R}_{n , j} ( θ ) \vert \leq {K ( {\sum }_{i = 1}^{p} \vert {θ}_{i} \vert )}^{2} \leq Kp {‖ θ ‖}^{2} .$$

Then, for n≥n₁, with probability at least 1−η, from (10), (14) and (12),

$$‖ {U}_{n} ( θ ) - Γ ( θ ) ‖ \leq ‖ {U}_{n} ( θ ) - {U}_{{n}^{′}} ( 0 ) θ ‖ + ‖ {U}_{{n}^{′}} ( 0 ) θ - Γθ ‖ = ‖ {U}_{n} ( 0 ) + \frac{1}{2} {R}_{n} ( θ ) ‖ + ‖ ( {U}_{{n}^{′}} ( 0 ) - Γ ) θ ‖ < {δ}^{2} + \frac{{Kp}^{3 / 2}}{2} {‖ θ ‖}^{2} + δ ‖ θ ‖ \leq C {δ}^{2} . \qquad ( 15 )$$

$$So ‖ {θ}^{T} {U}_{n} ( θ ) - {θ}^{T} Γθ ‖ < C {δ}^{3} .$$

$$Hence , if ‖ θ ‖ = δ , {θ}^{T} {U}_{n} ( θ ) > {θ}^{T} Γθ - C {δ}^{3} \geq {γδ}^{2} - C {δ}^{3} = {δ}^{2} ( γ - C δ ) > 0.$$

Assume for the sake of contradiction that Uₙ(θ) does not have a root in B_{δ}. Then for θ∈B_{δ}, the function ƒ(θ)=−δUₙ(θ)/∥Uₙ(θ)∥ continuously maps B_{δ} to itself. By the Brouwer fixed point theorem, there exists ϑ∈B_{δ}, with ƒ(ϑ)=ϑ. Since ∥ƒ(θ)∥=δ for all θ∈B_{δ}, we have ∥ƒ(ϑ)∥=∥ϑ∥=δ, contradicting (15) via δ²=∥ϑ∥²=ϑ^{T}ϑ=ϑ^{T}ƒ(ϑ)<0. Hence Uₙ(θ) has a root within δ of 0, and since δ<ϵ, therefore within ϵ, with probability at least 1−η, as required.

To prove (13), taking \hat{θ}ₙ to be any consistent sequence for θ₀, a first order Talyor expansion yields, for all 1≤j, k≤p,

$$\begin{matrix}{\partial }_{k} {U}_{n , j} ( {\hat{θ}}_{n} ) = {\partial }_{k} {U}_{n , j} ( 0 ) + {\sum }_{i = 1}^{p} {\partial }_{k , l} {U}_{n , j} ( {θ}_{n , j}^{*} ) {\hat{θ}}_{n , i} \\ = {\partial }_{k} {U}_{n , j} ( 0 ) + {Q}_{k , n , j}^{r} {\hat{θ}}_{n}\end{matrix}$$

$$where {Q}_{k , n , j}^{T} := ( {\partial }_{k , 1} {U}_{n , j} ( {θ}_{n , j}^{*} ) , … , {\partial }_{k , p} {U}_{n , j} ( {θ}_{n , j}^{*} ) ) ,$$

where θ*_{n,j} lies along the line segment connecting \hat{θ}ₙ and 0. Writing this identity in matrix notation, we have

U_{n′}(\hat{θ}ₙ)−U_{n′}(0)=Qₙ(\hat{θ}ₙ) where (Qₙ(θ))_{k,j}=Q_{n,k,j}^{T}θ.

Let η∈(0,1) and ϵ>0 be given, choose δ∈(0, ϵ/K \sqrt{p}) so that B_{δ}⊂Θ₀, and let n₂ be such that for all n≥n₂, with probability at least 1−η, |∂_{k,l}Uₙ(θ)|≤K for all 1≤k,l≤p and ∥\hat{θ}ₙ∥≤δ.

Then, for n≥n₂ with probability at least 1−η we have

$$\vert {Q}_{n , k , j}^{T} {\hat{θ}}_{n} \vert \leq K \sqrt{p} δ < ϵ , orequivalently , {‖ {U}_{{n}^{′}} ( {\hat{θ}}_{n} ) - {U}_{{n}^{′}} ( 0 ) ‖}_{∞} < ϵ$$

where ∥A∥_{∞}=max_{i,j}|A_{i,j}| for A∈ [symbol] ^{p×p}. The claim follows, since ϵ and η are arbitrary, and U_{n′}(0)→ₚΓ by assumption. Our next result provides conditions under which a consistent estimator sequence, properly centered and scaled, converges in distribution.

Theorem 2.2 Suppose the sequence of solutions \hat{θ}ₙ, n≥1 to (7) is consistent for θ₀, that (12) and the second condition of (11) hold for some sequence aₙ, n≥1 of real numbers, that the matrix Γ in (11) is non-singular and that Uₙ(θ) is twice continuously differentiable in an open set Θ₀⊂Θ containing θ₀. Further, let bₙ be a sequence of real numbers such that for some random variable Y,

$${b}_{n} {U}_{n} ( {θ}_{0} ) {\to }_{d} Y . \qquad ( 16 )$$

$$Then$$

$$\frac{{b}_{n}}{{a}_{n}} ( {\hat{θ}}_{n} - {θ}_{0} ) {\to }_{d} - {Γ}^{- 1} Y .$$

Proof: As in the proof of Theorem 2.1, by replacing aₙ [symbol] ₙ by [symbol] ₙ we may without loss of generality take aₙ=1, and also as done there, take θ₀=0. Since a limit in distribution does not depend on events of vanishingly small probability, by the consistency of \hat{θ}ₙ and (12) we may assume that for each n, sufficiently large, that \hat{θ}ₙ∈Θ₀, and for some K that |∂_{k,j}Uₙ(θ)|≤K for all 1≤j, k≤p and θ∈Θ₀. For such n the expansion (10) holds, and substituting \hat{θ}ₙ for θ and using Uₙ(\hat{θ}ₙ)=0 yields

$$- {U}_{n} ( 0 ) = ( {U}_{{n}^{′}} ( 0 ) + {ϵ}_{n} ) {\hat{θ}}_{n} := {Γ}_{n} {\hat{θ}}_{n} where$$

$${( {ϵ}_{n} )}_{j , l} = \frac{1}{2} {\sum }_{k = 1}^{p} {\hat{θ}}_{n , k} {\partial }_{k , l} {U}_{n , j} ( {θ}_{n , j}^{*} ) .$$

By the Cauchy-Schwarz inequality,

$$\vert {( {ϵ}_{n} )}_{j , l} \vert \leq \frac{K \sqrt{p}}{2} ‖ {\hat{θ}}_{n} ‖ {\to }_{p} 0.$$

Hence Γₙ→ₚ so that Γₙ⁻¹ exists with probability tending to 1, and converges in probability to Γ⁻¹. Now using (16), Slutsky's theorem, on an event of probability tending to one as n tends to infinity,

bₙ\hat{θ}ₙ=Γₙ⁻¹(bₙΓₙ\hat{θ}ₙ)=−Γₙ⁻¹(bₙUₙ(0))→_{d}−Γ⁻¹Y.

In the most common case the distributional convergence in (16) is to the normal, and shown by applying the Central Limit Theorem to a sum of independent random vectors. This situation is illustrated in the following lemma, in which we include distributional limits that may have covariance matrices of less than full rank. For a given vector μ and non-negative definite matrix Σ,

we say X˜N(μ, Σ) when E[e^{t}^{^{T}}^{X}]=exp(½t^{T}Σt+t^{T}μ).

In particular, in one dimension N(μ, 0) is unit mass at μ.

Lemma 2.1 Let [symbol] =1,2, . . . be a sequence of arbitrary index sets satisfying | [symbol] |→∞ as [symbol] →∞, and let { [symbol] , a∈ [symbol] } be a collection of [symbol] ^{d} valued independent, mean zero random vectors such that for some matrix Σ and some η>0

$${lim}_{ℓ \to ∞} {\sum }_{a ∈ {𝒜}_{ℓ}} Var ( {X}_{ℓ , a} ) = Σ and {lim}_{ℓ \to ∞} {\sum }_{a ∈ {𝒜}_{ℓ}} 𝔼 {‖ {X}_{ℓ , a} ‖}^{2 + η} = 0. \qquad ( 17 )$$

$$Then {S}_{ℓ} = {\sum }_{a ∈ {𝒜}_{ℓ}} {X}_{ℓ , a} satisfies {S}_{ℓ} \to N ( 0 , Σ ) as ℓ \to ∞ .$$

Proof: We first prove the result in [symbol] . By the Lindeberg theorem, (e.g. Theorem 3.4.5, [Durrett, 2019]) if for all [symbol] ≥1 the random variables { [symbol] , a∈ [symbol] ᵢ} are independent, mean zero, and satisfy

$${lim}_{ℓ \to ∞} {\sum }_{a ∈ {𝒜}_{ℓ}} Var ( {X}_{ℓ , a} ) = {σ}^{2} > 0 , and for all ϵ > 0 \qquad ( 18 )$$

$${lim}_{ℓ \to ∞} {\sum }_{a ∈ {𝒜}_{ℓ}} ‖ {X}_{ℓ , a}^{2} 1 ( \vert {X}_{ℓ , a} \vert \geq ϵ ) ] = 0 ,$$

then [symbol] →_{d}N(0, σ²) where [symbol] = [symbol] X_{n,i}. In [symbol] the second condition in (17) implies the second condition in (18), as for any ϵ>0, with p=1+η/2 and q=1+2/η, using Hölder's inequality followed by Markov's,

$$E [ {X}_{ℓ , a}^{2} 1 ( \vert {X}_{ℓ , a} \vert \geq ϵ ) ] \leq {E [ {X}_{ℓ , a}^{2 p} ]}^{1 / p} {P ( \vert {X}_{ℓ , a} \vert \geq ϵ )}^{1 / q} \leq {E [ {X}_{ℓ , a}^{2 p} ]}^{1 / p} {( \frac{E [ {X}_{ℓ , a}^{2 p} ]}{{ϵ}^{2 p}} )}^{1 / p} = \frac{E [ {X}_{ℓ , a}^{2 p} ]}{{ϵ}^{2 p / q}} = \frac{E [ {X}_{ℓ , a}^{2 + η} ]}{{ϵ}^{2 p / q}} .$$

Hence, the claim holds in [symbol] when the limiting variance is positive. When this limit is zero, Chebyshev's inequality yields that [symbol] →ₚ0, and hence [symbol] converges as well to zero in distribution, which is the normal distribution with mean and variance 0. Hence the conclusion of the lemma holds for d=1.

In general, given a collection of random vectors satisfying the given hypotheses, taking v to be of norm 1, the variables [symbol] =v^{T} [symbol] for a∈ [symbol] are independent and mean zero for each [symbol] , and satisfy the first condition of (17) with Σ replaced by v^{T}Σv, and the second condition of (17) by virtue of this condition holding by assumption for the vector array [symbol] . and that | [symbol] |^{2+η}=|v^{T} [symbol] |^{2+η}≤∥ [symbol] ∥^{2+η}. As the claim holds in d=1 for linear combinations given by any v of norm 1, the general result follows by the Cramer-Wold device.

Examples (Section 2.3). In the section we demonstrate the scope of the results in Section 2.2 by presenting two applications, one to least squares and the other to maximum likelihood.

The following lemma, a direct application of the dominated convergence theorem, is used to handle the technical matter of interchanges between integration and differentiation with respect to θ∈Θ⊂ [symbol] ^{p}.

Lemma 2.2 Let ƒ: [symbol] ^{m}×Θ→ [symbol] be differentiable with respect to θ in an open set Θ₀⊂Θ, and suppose that there exists g: [symbol] ^{m}→ [symbol] such that

$$\vert \frac{\partial }{{\partial }_{θ}} f ( x ; θ ) \vert \leq g ( x ) for all θ ∈ {Θ}_{0} and {\int }_{{ℝ}^{m}} g ( x ) dx < ∞ .$$

Then for all θ∈Θ₀,

$$\frac{\partial }{{\partial }_{θ}} {\int }_{{ℝ}^{m}} f ( x ; θ ) dx = {\int }_{{ℝ}^{m}} \frac{\partial }{{\partial }_{θ}} f ( x ; θ ) dx . \qquad ( 19 )$$

Example 2.1 Least squares estimation. Suppose we observe

yᵢ=ƒ(xᵢ, θ₀)+ϵᵢ i=1, . . . , n

where ƒ(xᵢ, θ), θ∈Θ⊂ [symbol] is some specified parametric family of functions; we take a one dimensional parameter here for simplicity. We estimate θ₀ via least squares, minimizing

$${J}_{n} ( θ , {x}^{( n )} ) = \frac{1}{2 n} {\sum }_{i = 1}^{n} {( f ( {x}_{i} , θ ) - {y}_{i} )}^{2} = \frac{1}{2 n} {\sum }_{i = 1}^{n} {( f ( {x}_{i} , θ ) - f ( {x}_{i} , {θ}_{0} ) - {ϵ}_{i} )}^{2} .$$

We assume that ƒ(x, θ) has three continuous derivatives with respect to θ that are uniformly bounded, say by K, over some open subset Θ₀ of Θ that contains θ₀, and that ϵ₁, ϵ₂, . . . are independent random variable distributed as ϵ, a mean zero, variance σ² random variable E|ϵ|^{2+η}=τ^{2+η}<∞ for some η>0.

Taking one derivative with respect to θ, we obtain the estimating equation [symbol] ₙ(θ)=0 where

$${𝒰}_{n} ( θ ) = \frac{1}{n} {\sum }_{i = 1}^{n} ( f ( {x}_{i} , θ ) - f ( {x}_{i} , {θ}_{0} ) - {ϵ}_{i} ) {\partial }_{θ} f ( {x}_{i} , θ ) \qquad ( 20 )$$

$$so in particular {𝒰}_{n} ( {θ}_{0} ) = - \frac{1}{n} {\sum }_{i = 1}^{n} {ϵ}_{i} {\partial }_{θ} f ( {x}_{i} , {θ}_{0} ) .$$

The first condition of (11) of Theorem 2.1 is satisfied with aₙ=1, as the errors have zero mean, are uncorrelated and have uniformly bounded variances, implying that E_{θ}_{₀}[ [symbol] ₙ(θ₀)]=0 and Var_{θ}_{₀}[ [symbol] ₙ(θ₀)]→0. Regarding the second condition of (11) taking another derivative, we obtain

$$\begin{matrix}{𝒰}_{{n}^{′}} ( {θ}_{0} ) = \frac{1}{n} {\sum }_{i = 1}^{n} ( {( {\partial }_{θ} f ( {x}_{i} , θ ) )}^{2} + ( f ( {x}_{i} , θ ) - f ( {x}_{i} , {θ}_{0} ) - {ϵ}_{i} ) {\partial }_{θ}^{2} f ( {x}_{i} , θ ) ) \\ = \frac{1}{n} {\sum }_{i = 1}^{n} {( {\partial }_{θ} f ( {x}_{i} , {θ}_{0} ) )}^{2} - \frac{1}{n} {\sum }_{i = 1}^{n} {ϵ}_{i} {\partial }_{θ}^{2} f ( {x}_{i} , {θ}_{0} ) .\end{matrix} \qquad ( 21 )$$

Arguing as for (20), the second sum tends to zero in probability. If we now take xᵢ, i=1,2, . . . to be independent random vectors distributed as some x, then the law of large numbers yields that

$$\frac{1}{n} {\sum }_{i = 1}^{n} {( {\partial }_{θ} f ( {x}_{i} , {θ}_{0} ) )}^{2} {\to }_{p} γ = {{E}_{{θ}_{0}} ( {\partial }_{θ} f ( {x}_{i} , {θ}_{0} ) )}^{2} , \qquad ( 22 )$$

showing the second condition of (11), and this limit will be positive when ∂_{θ}ƒ(x, θ₀) is a non-degenerate random variable, thus verifying the final condition in (11) in that case.

It is easy to see that taking another derivative in (21) yields an average of functions that are bounded over Θ₀, plus a weighted average of the error variables, each one multiplied by some bounded function. As the second weighted average can be seen to be bounded in probability by applying reasoning similar to that used for the score [symbol] ₙ(θ₀), condition (12) holds.

The only remaining verification needed to invoke Theorem 2.2 is to show the properly scaled score at θ₀ has a limiting distribution. Taking bₙ=\sqrt{n}, we have

$$Var ( \frac{1}{\sqrt{n}} {𝒰}_{n} ( {θ}_{0} ) ) \to {σ}^{2} γ ,$$

by (22), and in addition using the representation of [symbol] ₙ(θ₀) from (20),

$${\sum }_{i = 1}^{n} 𝔼 {\vert \frac{{ϵ}_{i} {\partial }_{θ} f ( {x}_{i} , {θ}_{0} )}{\sqrt{n}} \vert}^{2 + η} \leq {K}^{2 + η} {n}^{- η / 2} {τ}^{2 + η} \to 0.$$

Hence, invoking Lemma 2.1, for any consistent sequence of roots,

\sqrt{n}(\hat{θ}₀−θ₀)→_{d}N(0, σ²γ⁻¹).

Example 2.2 Maximum likelihood. Let p(x, θ), θ∈Θ₀ be a family of density functions for Θ⊂ [symbol] ^{p}, and for some θ₀∈Θ, let X₁, . . . , Xₙ be independent random vectors with density p(x, θ₀). Let p(x, θ) be three times continuously differentiable in θ with the first two derivatives of p(x, θ), and the third derivative of q(x,θ)=log p(x,θ), dominated by an integrable function in some neighborhood Θ₀ of θ₀. Assume further that the Fisher information matrix at θ₀ is positive definite.

The maximum likelihood estimate of θ₀ is obtained by maximizing the log likelihood of the data, and hence given by a solution to the estimating equation (7) with

$${𝒰}_{n} ( θ ) = \frac{1}{n} {\sum }_{i = 1}^{n} \frac{{\partial }_{θ} p ( {X}_{i} , θ )}{p ( {X}_{i} , θ )}$$

By Lemma 2.2, for θ∈Θ₀ we have

[symbol]

_{θ}[∂_{θ} log p(X, θ)]= [symbol] ∂_{θ}p(x, θ)dx=∂_{θ} [symbol] p(x, θ)dx=0, (23)

and likewise that the Fisher information I(θ) satisfies

l(θ=− [symbol] _{θ}[∂_{θ}²log p(X, θ)]=Var_{θ}(∂_{θ}log p(X, θ)).

Hence, by the law of large numbers the first two conditions of (11) are satisfied with aₙ=1 and Γ=(θ₀), and the last holds by our assumption on the Fisher information. Next we show (12) is satisfied. Writing ∂ⱼ short for ∂_{θ}_{ⱼ}, we may write

$${𝒰}_{n} ( θ ) = {𝒰}_{n} ( θ ) = {\partial }_{θ} q ( {X}_{i} , θ ) andhence {\partial }_{k , l} {𝒰}_{n , j} ( θ ) = \frac{1}{n} {\sum }_{i = 1}^{n} {\partial }_{k , l , j} q ( {X}_{i} , θ ) .$$

Condition (12) can be verified by invoking the following uniform strong law of large numbers with h(x, θ) applied to the components ∂_{k,i,j}q(x, θ).

Theorem 2.3 Let Θ be a compact metric space and χ a space on which a probability distribution F is defined. Let h(x, θ) be measurable in x for each θ∈Θ and continuous in θ for almost every x. Assume there exists K(x) such that E[K(X)]<∞ and |h(x, θ)|≤K(x) for all x and θ. Then, with m(θ)=E[h(X, θ)].

$$P ( {lim}_{n \to ∞} {sup}_{θ ∈ Θ} \vert \frac{1}{n} {\sum }_{i = 1}^{n} h ( {X}_{i} , θ ) - m ( θ ) \vert = 0 ) = 1 ,$$

where X₁, X₂, . . . are independent with distribution F.

Lastly, under the given assumptions, the classical central limit theorem yields

\sqrt{n} [symbol] ₙ(θ₀)→_{d} [symbol] (0, I(θ₀))

so that, via Theorem 2.2.

\sqrt{n}(\hat{θ}₀−θ₀)→_{d} [symbol] (0, I(θ₀)⁻¹).

For the exponential family

p(x; θ)=h(x)exp(η(θ)T(x)−A(θ)) we have q(x; θ) =log h(x)+η(θ)T(x)−A(θ).

Hence, the needed conditions are satisfied if A(θ) and η(θ) have three bounded derivatives in some neighborhood of θ₀, and E_{θ}_{₀}[T(X)] exists.

Application to a diffusion equation model (Section 3). To more fully specify the output function of the diffusion model arising from PDE as described herein, consider the parameter space

[symbol]

={(q₁, q₂)∈ [symbol] ²:q₂>0}, (24)

and for given matrices D, E∈ [symbol] ^{k×k}, a vector F∈ [symbol] ^{k} and q∈ [symbol] , recall from (5) that

A=A(q)=q₁D+E, B=B(q)=q₂F, (25)

and that the TAC at time t is given by

ƒ_{μ}(t; q)=∫₀^{t}Ce^{A(t-s)}Bμ(s)ds, (26)

where C^{T}∈ [symbol] ^{k}, and μ(s) is the BrAC/BAC at time s. Though our methods work in the given generality, in the physics based model the matrix A will have eigenvalues with negative real parts, and q₁ will be strictly positive. The dependence of ƒ on A, B, C, μ or q may be dropped in the following for ease of notation, or included to emphasize some particular feature of interest.

Consider an individual whose data has been collected over i=1, . . . , n drinking sessions, where the BrAC curve μᵢ for episode i is integrable on [0, Tᵢ], and for some q₀∈ [symbol] and mᵢ observations of TAC plus a mean zero error

yᵢⱼ=ƒ_{μ}_{ᵢ}(t_{i,j}; q₀)+ϵ_{i,j}, (27)

are taken at the times 0≤t_{i,1}≤ . . . ≤t_{i,j}_{ᵢ}≤Tᵢ≤T, for some T>0. For notational simplicity we may suppress some of the parameters in (27), for instance, denoting ƒ_{μ}_{ᵢ}(t_{i,j}; q) by ƒᵢⱼ(q), say. We encode the observation times of episode i as the probability measure putting mass 1/mᵢ on each observation time, and form the vector of probability measures vₙ=(v_{1,m}_{₁}, . . . , v_{n,m}_{ₙ}). When n=1, that is, for the case of a single episode, we drop the index l.

For asymptotics, we consider a sequence of experiments indexed by [symbol] =1,2, . . . , where n and m=(m₁, . . . , mₙ) may depend on [symbol] , and hence we may index using [symbol] in place of n, m, though this dependence may at times be suppressed in the notation. In the case of a single drinking episode, that is, when n=1, we let [symbol] =m. For consistency and asymptotic normality, we require that

Σᵢ₌₁ⁿmᵢ→∞ as [symbol] →∞. (28)

In the special case where the number of observations mᵢ for each n equals a constant m, the requirement (28) becomes nm→∞, and in the sub-case of a single drinking episode. that m→∞.

Recall that a sequence of measures {vₖ, k≥1} on [symbol] is said to converge weakly to a measure v if

$${min}_{k \to ∞} {\int }_{ℝ} g ( u ) {dv}_{k} = {\int }_{ℝ} g ( u ) dv$$

$$for all bounded continuous functions g : ℝ \to ℝ .$$

Any sequence {vₖ, k≥1} of probability measures whose supports are contained in a bounded set is tight, and hence when the weak limit v exists it will also be a probability measure, and its support also so contained.

There are two special cases of note for the sequence of measures {vₖ, k≥1}. One is where the distances between consecutive observation times on [0, T] are constant; in this case, the weak limit is the uniform probability measure on [0, T]. A second case is when the observation times are chosen independently according to the probability measure v supported on [0, T]; in this case, the weak limit in probability is μ.

Let the gradient of [symbol] (q) be denoted

$${\partial }_{q} {J}_{ℓ} ( q ) = ( \begin{matrix}{\partial }_{1} {J}_{ℓ} ( q ) \\ {\partial }_{2} {J}_{ℓ} ( q )\end{matrix} ) and let \qquad ( 29 )$$

$${g}_{μ} ( u ; q ) = {\partial }_{q} {f}_{μ} ( u ; q ) {\partial }_{q} {{f}_{μ} ( u ; q )}^{⊤} .$$

We apply the methods developed herein to the least squares estimator achieved as a solution to

[symbol]

(q)=0 where [symbol] (q)=∂_{q} [symbol] (q), (30)

where [symbol] (q) is given by the sum of squares in (6). For i∈{1,2}, we continue to let ∂ᵢ denote taking the partial derivative with respect to qᵢ; this notation will extend in the natural way to denote higher order, and mixed partial derivatives. Theorem 3.1 below gives conditions under which the least squares estimate is consistent and has a limiting, asymptotically normal distribution, and as well provides the form of the limiting covariance matrix. Theorem 3.1 is an immediate consequence of Theorems 3.2 and 3.3. that verify the conditions of Theorems 2.1 and 2.2 in the previous section.

To set the stage for the statements and proofs of our results, we note that when vₙ, m≥1 is the discrete probability measure giving equal weight to the times t_{m,1}, . . . , t_{m,m} in [0, T], then for any continuous function h:[0, T]→ [symbol] , when vₘ converges weakly to v, we have

$$\frac{1}{m} {\sum }_{j = 1}^{m} h ( {t}_{m , j} ) = {\int }_{0}^{T} h ( u ) {dv}_{m} \to {\int }_{0}^{T} h ( u ) dv . \qquad ( 31 )$$

By considering components, the same relations hold when h continuously maps [0, T] to the space of matrices of some fixed dimension. For a given BrAC curve μ, of particular interest is the [symbol] ^{2×2} valued matrix function g_{μ} in (29) that determines, via [symbol] (q), the limiting covariance matrix Γ of our q parameter estimate.

We consider two special cases where the existence of the limit Γ is guaranteed. For a single drinking episode, that is, when n=1, when vₘ converges weakly to v, due to the continuity of elements of g_{μ}(u) as shown in Lemma 3.3, we have, as m→∞,

$$\begin{matrix}{Γ}_{m} = {\int }_{0}^{T} {g}_{μ} ( u ) {dv}_{m} \to {\int }_{0}^{T} {g}_{μ} ( u ) dv \\ = ( \begin{matrix}{\int }_{0}^{T} {( {\partial }_{1} {f}_{μ} ( u ) )}^{2} dv & \frac{1}{{q}_{0.2}} {\int }_{0}^{T} {f}_{μ} ( u ) {\partial }_{1} {f}_{μ} ( u ) dv \\ \frac{1}{{q}_{0.2}} {\int }_{0}^{T} {f}_{μ} ( u ) {\partial }_{1} {f}_{μ} ( u ) dv & \frac{1}{{q}_{0.2}^{2}} {\int }_{0}^{T} {{f}_{μ} ( u )}^{2} dv\end{matrix} ) \\ = Γ\end{matrix} \qquad ( 32 )$$

In particular, v will be the uniform probability measure on [0, T] when the number m of sampling times tend to infinity, and the consecutive distances between them are equal.

For another case, consider a situation where the data from n drinking episodes are independent and identically distributed from replicates of the error distribution and canonical M, T, μ, v, where M is the distribution of mᵢ, 1≤i≤n, making the summands in (33) i.i.d. When T<∞a.s. Lemma 3.3 shows that the integrals in (33) are uniformly bounded, and one can show that as n→∞.

$${Γ}_{n} {\to }_{p} Γ = E [ \frac{M}{E [ M ]} ( \begin{matrix}{\int }_{0}^{T} {( {\partial }_{1} {f}_{μ} ( u ) )}^{2} dv & \frac{1}{{q}_{0.2}} {\int }_{0}^{T} {f}_{μ} ( u ) {\partial }_{1} {f}_{μ} ( u ) dv \\ \frac{1}{{q}_{0.2}} {\int }_{0}^{T} {f}_{μ} ( u ) {\partial }_{1} {f}_{μ} ( u ) dv & \frac{1}{{q}_{0.2}^{2}} {\int }_{0}^{T} {{f}_{μ} ( u )}^{2} dv\end{matrix} ) ] ,$$

where the expectation is taken over M, T, μ and v, whenever the expectation on the right hand side exists. We now present our main result regarding the least squares estimator for the diffusion model.

Theorem 3.1 Suppose the errors ϵ_{i,j}, 1≤i≤n. 1≤j≤mᵢ in model (27) are mean zero, uncorrelated and have constant positive variance σ². With μᵢ and v the BrAC curve and the empirical measure of the observation times for episode i=1, . . . , n, we assume the existence of the limit

$$Γ = {lim}_{ℓ \to ∞} {Γ}_{ℓ} where \qquad ( 33 )$$

$${Γ}_{ℓ} = {\sum }_{i = 1}^{n} \frac{{m}_{i}}{{\sum }_{k = 1}^{n} {m}_{k}} {\int }_{0}^{{T}_{i}} {\partial }_{q} {f}_{{μ}_{i}} ( u ; {q}_{0} ) {\partial }_{q} {{f}_{{μ}_{i}} ( u ; {q}_{0} )}^{⊤} {dv}_{i} .$$

that Γ is positive definite, and that (28) holds. Then there exists a consistent sequence of solutions [symbol] to the estimating equation [symbol] (q)=0.

If in addition the errors ϵ_{i,j} are i.i.d., and for some η>0 satisfy E|ϵ_{i,j}|^{2+η}=τ^{2+η}<∞, then, along any such consistent sequence [symbol] ,

$$\sqrt{m} ( {\hat{q}}_{ℓ} - {q}_{0} ) {\to }_{d} 𝒩 ( 0 , {σ}^{2} {Γ}^{- 1} ) where m = {\sum }_{i = 1}^{n} {m}_{i} and \qquad ( 34 )$$

$${\hat{σ}}_{ℓ}^{2} {\to }_{p} {σ}^{2} where {\hat{σ}}_{ℓ}^{2} = \frac{1}{m} {\sum }_{i = 1}^{n} {\sum }_{j = 1}^{{m}_{i}} {( {y}_{ij} - {f}_{ij} ( {\hat{q}}_{ℓ} ) )}^{2} .$$

When the errors ϵ_{i,j} in (27) are Gaussian, then the least squares estimate that minimizes the sum of squares (6) is also maximum likelihood. In this case the contribution to the Fisher information from the single observation in (27) is obtained by taking the covariance matrix of the gradient of the log of the density of the observation,

$$Var ( {\partial }_{q} log p ( {y}_{ij} ; {f}_{ij} ) ) = Var ( {\partial }_{q} log ( \frac{1}{\sqrt{2 π} σ} exp ( - \frac{1}{2 {σ}^{2}} {( {y}_{ij} - {f}_{ij} )}^{2} ) ) ) = Var ( {\partial }_{q} ( - \frac{1}{2 {σ}^{2}} {( {y}_{ij} - {f}_{ij} )}^{2} ) ) = Var ( \frac{{ϵ}_{ij}}{{σ}^{2}} {\partial }_{q} {f}_{ij} ) = \frac{1}{{σ}^{2}} {\partial }_{q} {f}_{ij} {\partial }_{q} {f}_{ij}^{⊤} .$$

Summing over the observation times yields m [symbol] /σ² as in (33), hence taking the limit and comparing with the asymptotic variance obtained we see that for normal errors the least squares estimate of q achieves the lower bound of the information inequality in an asymptotic sense.

Before proceeding, we must verify the smoothness of the derivatives of ƒ_{μ}(t; q) in (26) with respect to q=(q₁, q₂). Because of the form of the dependence of the matrix A on q₁ as given in (25), to differentiate ƒ with respect to q₁ we will need to consider directional derivatives of matrix exponentials. For square matrices W and V of the same dimension and u∈ [symbol] , define the first derivative of e^{uW} in direction V by

$${𝒟}_{V}^{1} ( u , W ) = {lim}_{h \to 0} \frac{exp ( u ( W + hV ) ) - exp ( uW )}{h} .$$

We define higher order derivatives [symbol] _{V}^{k}(u, W), k≥0 in the natural way, with k=0 returning e^{uW}. Now with A=q₁D+E as in (25), we may represent the partial derivative with respect to q₁ of e^{uA} as

$${\partial }_{1} {e}^{uA} = {\partial }_{1} {e}^{u ( {q}_{1} D + E )} = {lim}_{h \to 0} \frac{{e}^{u ( ( {q}_{1} + h ) D + E )} - {e}^{u ( {q}_{1} D + E )}}{h} = {lim}_{h \to 0} \frac{{e}^{u ( A + hD )} - {e}^{uA}}{h}$$

$$= {𝒟}_{D}^{1} ( u , A ) ,$$

and extending to higher order derivatives we obtain

∂₁ⁿ(e^{(q}^{₁}^{D+E)μ)}= [symbol] _{D}ⁿ(u, A). (35)

For any n≥0, letting Bₙ be the (n+1)×(n+1) block matrix given by

$${B}_{n} = [ \begin{matrix}W & V & 0 & … & 0 \\ 0 & W & V & … & 0 \\ … & … & … & … & … \\ 0 & 0 & … & 0 & W \\ & & & & \end{matrix} ] , \qquad ( 36 )$$

A known theorem provides that

$${e}^{{uB}_{n}} = [ \begin{matrix}{e}^{uW} & \frac{{𝒟}_{V}^{1} ( u , W )}{1 !} & \frac{{𝒟}_{V}^{2} ( u , W )}{2 !} & … & \frac{{𝒟}_{V}^{n} ( u , W )}{n !} \\ 0 & {e}^{uW} & \frac{{𝒟}_{V}^{1} ( u , W )}{1 !} & … & \frac{{𝒟}_{V}^{n - 1} ( u , W )}{( n - 1 ) !} \\ … & … & … & … & … \\ 0 & 0 & … & 0 & {e}^{uW} \\ & & & & \end{matrix} ] . \qquad ( 37 )$$

We now apply (37) to obtain bounds on higher order derivatives of the matrix exponential e^{uA} with respect to q₁.

Lemma 3.1 Let W and V be square matrices of the same dimension. Then for all n≥0 the directional derivative [symbol] _{V}ⁿ(u, W) is analytic in u on [symbol] and satisfies the bound

∥ [symbol] _{V}ⁿ(u, W)∥≤n!∥e^{uB}^{ₙ}∥ for all u∈ [symbol] , (38)

where Bₙ is given by (36). For all n≥0, q₁∈ [symbol] and A=q,₁D+E, the partial derivative ∂₁ⁿe^{Au} exists, is analytic in q₁ and satisfies ∥∂₁ⁿe^{uA}∥≤n!e^{u∥B}^{_{n∥}} where Bₙ is given by (36) with W=A and V=D.

Proof: As the left hand side e^{uB}^{ₙ} of (37) is analytic in each component, the matrix on the right hand side must also be analytic, thus yielding the first claim. Next, for F the submatrix obtained by taking row and column indices i, j of a given matrix E, applying an alternate form for the spectral norm in the first equality, we have

$$‖ F ‖ = {sup}_{‖ y ‖ = 1 , ‖ x ‖ = 1} {y}^{⊤} Fx \leq {sup}_{‖ u ‖ = 1 , ‖ v ‖ = 1} {u}^{⊤} Ev = ‖ E ‖ ,$$

as any value over which the first supremum is taken can be achieved in the second by padding x and y with zeros in coordinates that are not in i and j, respectively. Hence, inequality (38) now follows from (37). The remaining claims now follow in light of (35).

We require the following result to handle the derivatives of matrix products. For k≥0, Q as in (24), we say a matrix M depending on (q, u)∈Q× [symbol] is k-smooth if for any 0≤j₁, j₂≤k, the mixed partials ∂₁^{j}^{₁}∂₂^{j}^{₂}M exist and are continuous for q∈ [symbol] , and for any bounded subsets [symbol] ⊂ [symbol] and I⊂ [symbol] ,

$${sup}_{( q , u ) ∈ D \times i , 0 \leq {j}_{1} , {j}_{2} \leq k} ‖ {\partial }_{1}^{{J}_{1}} {\partial }_{2}^{{J}_{2}} M ‖ < ∞ .$$

We say M is smooth if it is k-smooth for all k≥0.

Lemma 3.2 Let Mᵢ, i=1, . . . , d be matrices having dimensions such that we may form the product

$$M = {\prod }_{i = 1}^{d} {M}_{i} .$$

If M₁, . . . , M_{d} are k-smooth then so is M.

Proof: The proof follows directly from the multivariate Leibniz rule that expresses the derivative ∂₁^{j}^{₁}∂₂^{j}^{₂}Mᵢ for 0≤j₁, j₂≤k as a finite linear combination of products of derivatives of Mᵢ, each one with order no greater than k, and recalling that for conformable matrices ∥AB∥≤∥A∥∥B∥.

The next lemma provides us with additional smoothness estimates, and the forms of derivatives that later appear.

Lemma 3.3 For all u∈ [symbol] the matrix function e^{Au}B is smooth in q. If γ(⋅) is integrable on [0, T], then ƒ_{γ}(t; q) as in (26) is smooth in q, continuous for t∈[0, T] and satisfies

|ƒ_{γ}(t; q)|≤q₂e^{T(q}^{₁}^{∥D∥+∥E∥)}∥γ∥₁ and ∂₁^{j}^{₁}∂₂^{j}^{₂}ƒ_{γ}(t; q)=∫₀^{t}∂₁^{j}^{₁}∂₂^{j}^{₂}(Ce^{A(t-s)}B)γ(s)ds.

For q∈ [symbol] , and

∂₁(e^{Au}B)=∂₁(e^{Au})B and ∂₂(e^{Au}B)=q₂⁻¹e^{Au}B. (39)

Proof: That e^{Au} is smooth follows from Lemma 3.1, and one easily verifies the smoothness of B directly from (25); hence, the product is smooth by Lemma 3.2. Differentiation under the integral is then justified by the dominated convergence theorem, from which the smoothness of ƒ_{γ}(t; q) in q then follows; continuity for t∈[0, T] follows immediately from the integral representation (26). The claims in (39) follow by recalling that B=q₂F.

We now begin to verify the conditions of Theorems 2.1 and 2.2.

Theorem 3.2 Suppose the errors ϵ_{i,j}, 1≤i≤n, 1≤j≤mᵢ in model (27) are mean zero, uncorrelated and have constant positive variance σ². Assume in addition that the limit Γ in (33) exists and is positive definite, and that (28) holds. Then with [symbol] given by (??) and (6), the hypotheses of Theorem 2. 1 are satisfied with Γ as in (33), aₙ=1 and any bounded neighborhood Θ₀⊂ [symbol] of q₀.

Proof: Let Θ₀ be any bounded neighborhood of q₀. By Lemma 3.3, the partial derivatives of ƒᵢⱼ(q):=ƒ_{μ}(t_{i,j}; q) of (26) of all orders exist, and are continuous and uniformly bounded over Θ₀. Hence [symbol] is twice continuously differentiable, with uniformly bounded derivatives, over Θ₀.

Now write the score function as

$${𝒰}_{ℓ} ( q ) = {𝒱}_{ℓ , 1} ( q ) - {𝒱}_{ℓ , 2} ( q ) \qquad ( 40 )$$

$$where$$

$${𝒱}_{ℓ , 1} ( q ) = \frac{1}{{\sum }_{i = 1}^{n} {m}_{i}} {\sum }_{i = 1}^{n} {\sum }_{j = 1}^{{m}_{i}} {\partial }_{q} {f}_{ij} ( q ) ( {f}_{ij} ( q ) - {f}_{ij} ( {q}_{0} ) )$$

$$and {𝒱}_{ℓ , 2} ( q ) = \frac{1}{{\sum }_{i = 1}^{n} {m}_{i}} {\sum }_{i = 1}^{n} {\sum }_{j = 1}^{{m}_{i}} {\partial }_{q} {f}_{ij} ( q ) {ϵ}_{ij} .$$

In particular,

$${𝒰}_{ℓ} ( {q}_{0} ) = - \frac{1}{{\sum }_{i = 1}^{n} {m}_{i}} {\sum }_{i = 1}^{m} {\sum }_{j = 1}^{{m}_{i}} {\partial }_{q} {f}_{ij} ( {q}_{0} ) {ϵ}_{ij} . \qquad ( 41 )$$

Differentiating (40) and evaluating at q₀, we find

[symbol]

(q₀)= [symbol] (q₀)− [symbol] (q₀), (42)

where, using (33),

$${𝒱}_{ℓ , 1}^{′} ( {q}_{0} ) = \frac{1}{{\sum }_{i = 1}^{n} {m}_{i}} {\sum }_{i = 1}^{n} {\sum }_{j = 1}^{{m}_{i}} {\partial }_{q} {f}_{ij} ( {q}_{0} ) {\partial }_{q} {{f}_{ij} ( {q}_{0} )}^{⊤} = {Γ}_{l}$$

$$and {𝒱}_{ℓ , 2}^{′} ( {q}_{0} ) = \frac{1}{{\sum }_{i = 1}^{n} {m}_{i}} {\sum }_{i = 1}^{n} {\sum }_{j = 1}^{{m}_{i}} {\partial }_{q}^{2} {f}_{ij} ( {q}_{0} ) {ϵ}_{ij} .$$

To show the first condition in (11), note that [symbol] [ [symbol] ₙ(q₀)]=0 as the error variables have mean zero. Next, using that the error variables are uncorrelated and have constant variance yields that the covariance matrix [symbol] =Var( [symbol] (q₀)) is given by

$${Ψ}_{ℓ} = \frac{{σ}^{2}}{{( {\sum }_{i = 1}^{n} {m}_{i} )}^{2}} {\sum }_{i = 1}^{n} {\sum }_{j = 1}^{m} {\partial }_{q} {f}_{ij} ( {q}_{0} ) {\partial }_{q} {{f}_{ij} ( {q}_{0} )}^{⊤} = \frac{{σ}^{2}}{{\sum }_{i = 1}^{n} {m}_{i}} {Γ}_{ℓ} . \qquad ( 43 )$$

The claim follows by noting that ∂₂ƒ_{μ}(q)=(1/q₂)ƒ_{μ}(q) by Lemma 3.3, and that ∥Ψₙ∥→0 as Γₙ→Γ and Σᵢmᵢ→∞ by assumption. For the second condition in (11), we recognize that [symbol] _{n,1}′(q₀)=Γₙ, and can show that the components of [symbol] _{n,2}′(q₀) have mean zero and variance converging to zero, so that the sum of these two matrices tends to Γ in probability as n→∞. The matrix Γ is positive definite by assumption, so the last condition in (11) holds.

Lastly, we show that inequality (12) is satisfied. From the decomposition (40) we see that we may write ∂_{k,l}U_{n,r}(q) as a difference of the form

$${R}_{n} - {S}_{n} := \frac{1}{{\sum }_{i = 1}^{n} {m}_{i}} {\sum }_{i = 1}^{n} {\sum }_{j = 1}^{{m}_{i}} {g}_{1 , ij} ( q ) - \frac{1}{{\sum }_{i = 1}^{n} {m}_{i}} {\sum }_{i = 1}^{n} {\sum }_{j = 1}^{{m}_{i}} {g}_{2 , ij} ( q ) {ϵ}_{i , j}$$

for some functions g_{p,ij,}p=1,2, where, by Lemma 3.3 and the product rule, for some K₁

$${sup}_{q ∈ {Q}_{0} , p ∈ \{ 1 , 2 \}} \vert {g}_{p , ij} ( q ) \vert \leq {K}_{1} .$$

Hence, for the first component,

$$\vert {R}_{n} \vert = \vert \frac{1}{{\sum }_{i = 1}^{n} {m}_{i}} {\sum }_{i = 1}^{n} {\sum }_{j = 1}^{{m}_{i}} {g}_{1 , ij} ( q ) \vert \leq \frac{1}{{\sum }_{i = 1}^{n} {m}_{i}} {\sum }_{i = 1}^{n} {\sum }_{j = 1}^{{m}_{i}} {K}_{1} = {K}_{1} ,$$

while for the second component,

$$Var ( {S}_{n} ) \leq \frac{{σ}^{2}}{{( {\sum }_{i = 1}^{n} {m}_{i} )}^{2}} {\sum }_{i = 1}^{n} {\sum }_{j = 1}^{{m}_{i}} {K}_{1}^{2} \leq \frac{{σ}^{2} {K}_{1}^{2}}{{\sum }_{i = 1}^{n} {m}_{i}} \to 0.$$

Hence, for any η∈(0,1), by Chebyshev's inequality, we may pick K₂ such that P(|Sₙ|≥K₂)≤η/8 for all n≥1. Thus, setting K=K₁+K₂, we obtain, for all n≥1,

$$P ( \vert {R}_{n} - {S}_{n} \vert > K , q ∈ {Q}_{0} ) \leq P ( \vert {R}_{n} \vert + \vert {S}_{n} \vert > K , q ∈ {Q}_{0} ) \leq P ( {K}_{1} + \vert {S}_{n} \vert > {K}_{1} + {K}_{2} , q ∈ {Q}_{0} ) = P ( \vert {S}_{n} \vert > {K}_{2} , q ∈ {Q}_{0} ) \leq \frac{η}{8} .$$

The claim now follows by taking a union bound over the eight choices for k, l and r.

Theorem 3.3 Assume the errors ϵᵢⱼ, 1≤i≤n, 1≤j≤mᵢ are i.i.d with mean zero, variance σ² and for some η>0 we have E|ϵᵢⱼ|^{2+η}=τ^{2+η}<∞. Assume that (28) holds and that the limit Γ as given in (33) exists. Then for [symbol] ₙ(q),

$$\begin{matrix}{b}_{n} {𝒰}_{n} ( {q}_{0} ) {\to }_{d} 𝒩 ( 0 , {σ}^{2} Γ ) & where & {b}_{n} = \sqrt{{\sum }_{i = 1}^{n} {m}_{i}} .\end{matrix}$$

Proof: We verify (17) of Lemma 2.1. For the first condition there,

$${𝒰}_{n} ( {q}_{0} ) = \frac{1}{{\sum }_{i = 1}^{n} {m}_{i}} {\sum }_{i = 1}^{n} {\sum }_{j = 1}^{{m}_{i}} ( \begin{matrix}{\partial }_{1} {f}_{ij} ( {q}_{0} ) \\ {\partial }_{2} {f}_{ij} ( {q}_{0} )\end{matrix} ) {ϵ}_{ij} \qquad ( 44 )$$

we see that the mean of bₙ [symbol] ₙ(q₀) is zero, and by (33)

$$Cov ( {b}_{n} {𝒰}_{n} ( {q}_{0} ) ) = {σ}^{2} {\sum }_{i = 1}^{n} \frac{{m}_{i}}{{\sum }_{k = 1}^{n} {m}_{k}} {\int }_{0}^{{T}_{i}} ( \begin{matrix}{\partial }_{1} {{f}_{ij} ( {q}_{0} )}^{2} & {\partial }_{1} {f}_{ij} ( {q}_{0} ) {\partial }_{2} {f}_{ij} ( {q}_{0} ) \\ {\partial }_{1} {f}_{ij} ( {q}_{0} ) {\partial }_{2} {f}_{ij} ( {q}_{0} ) & {\partial }_{1} {{f}_{ij} ( {q}_{0} )}^{2}\end{matrix} ) {dv}_{i , n} = {σ}^{2} {Γ}_{n} \to {σ}^{2} Γ .$$

For the second condition of (17), write

$$\begin{matrix}{b}_{n} 𝒰 ( {q}_{0} ) = {\sum }_{i = 1}^{n} {\sum }_{j = 1}^{{m}_{i}} {X}_{ij} & where & {X}_{ij} = -\end{matrix} \frac{1}{\sqrt{{\sum }_{i = 1}^{n} {m}_{i}}} ( \begin{matrix}{\partial }_{1} {f}_{ij} ( {q}_{0} ) \\ {\partial }_{2} {f}_{ij} ( {q}_{0} )\end{matrix} ) {ϵ}_{ij} .$$

By the assumption [symbol] |ϵᵢⱼ|^{2+η}≤τ^{2+η} and Lemma 3.3 there exists C such that

$${\sum }_{1 \leq i \leq n , 1 \leq j \leq {m}_{i}} E {‖ {X}_{ij} ‖}^{2 + η} \leq \frac{C {τ}^{2 + η}}{{( {\sum }_{i = 1}^{n} {m}_{i} )}^{1 + η / 2}} ,$$

which tends to zero by (28).

We conclude this section with: Proof of Theorem 3.1: Theorems 3.2 and 3.3 show that the hypotheses of Theorems 2.1 and 2.2 are satisfied, yielding the claims for consistency and asymptotic normality. It remains to prove the claims on the consistency of the variance estimator. By (34), and letting m=Σᵢ₌₁ⁿmᵢ, we have

$${\hat{σ}}_{n}^{2} = \frac{1}{m} {\sum }_{i = 1}^{n} {\sum }_{j = 1}^{{m}_{i}} {( {ϵ}_{ij} + {f}_{ij} ( {q}_{0} ) - {f}_{ij} ( {\hat{q}}_{n} ) )}^{2} = \frac{1}{m} {\sum }_{i = 1}^{n} {\sum }_{j = 1}^{{m}_{i}} ( {ϵ}_{ij}^{2} + 2 {ϵ}_{ij} ( {f}_{ij} ( {q}_{0} ) - {f}_{ij} ( {\hat{q}}_{n} ) + ( {f}_{ij} ( {q}_{0} ) - {{f}_{ij} ( {\hat{q}}_{n} )}^{2} ) .$$

The first term tends to σ² in probability by the weak law of large numbers. To handle the second term, letting

$$\begin{matrix}R = \frac{1}{m} {\sum }_{i = 1}^{n} {\sum }_{j = 1}^{{m}_{i}} \vert {ϵ}_{ij} \vert & we have & E [ R ] =\end{matrix} \frac{1}{m} {\sum }_{i = 1}^{n} {\sum }_{j = 1}^{{m}_{i}} E \vert {ϵ}_{ij} \vert \leq \frac{1}{m} {\sum }_{i = 1}^{n} {\sum }_{j = 1}^{{m}_{i}} \sqrt{E {ϵ}_{ij}^{2}} = σ .$$

With B₁ the unit ball centered at q₀, Lemma 3.3 shows that the first derivatives of ƒⱼ(q) are uniformly bounded for (q, t)∈B₁×[0, T], that is, there exists some K>0 such that over this set

|ƒ_{i j}(q₀)−ƒ_{i j}(q)|≤K∥q₀−q∥. (45)

Let δ∈(0, K) be arbitrary, F={|q₀−\hat{q}ₙ|≤δ/K} and note that

$$S = \vert \frac{1}{m} {\sum }_{i = 1}^{n} {\sum }_{j = 1}^{{m}_{i}} {ϵ}_{ij} ( {f}_{ij} ( {q}_{0} ) - {f}_{ij} ( {\hat{q}}_{n} ) \vert \qquad satisfies \qquad S {1}_{F} \leq δ R {1}_{F} .$$

By Markov's inequality, for any τ>0,

$$P ( S \geq τ ) \leq P ( S {1}_{F} \geq τ ) + P ( {F}^{c} ) \leq P ( δ R {1}_{F} \geq τ ) + P ( {F}^{c} ) \leq \frac{δ E [ R {1}_{F} ]}{τ} + P ( {F}^{c} ) \leq \frac{δ E [ R ]}{τ} + P ( {F}^{c} ) \leq \frac{δσ}{τ} + P ( {F}^{c} ) \to \frac{δσ}{τ} ,$$

using the non-negativity of R in the fourth inequality, and the consistency of qₙ when taking the limit. As δ can be made arbitrarily small we conclude that P(S≥τ)→0, and as t is arbitrary, that S→ₚ0.

Similarly decomposing the third term on the good event where qₙ is in B₁, and the complentary event which tends in probability to zero, on the good event, applying the inequality (45), this last term is bounded as

$$\frac{{K}^{2}}{m} {\sum }_{i = 1}^{n} {\sum }_{j = 1}^{{m}_{i}} {‖ {q}_{0} - {\hat{q}}_{n} ‖}^{2} = {K}^{2} {‖ {q}_{0} - {\hat{q}}_{n} ‖}^{2} ,$$

which tends to zero in probability in view of the consistency of \hat{q}ₙ.

Inference on the BrAC curve. In this section we obtain confidence bounds on a BrAC curve generated by a drinking episode of a subject in the field, and estimated using n TAC observations and an estimate qₘ computed from m measurements in a previous calibration experiment. Our notation here differs from that used in previous sections, the previous parameter n now being absorbed in the number m of total observations for calibration. Our uniform confidence bounds for the reconstructed BrAC curve are obtained by applying a variation on the standard multivariate delta method, using the properties provided by Theorem 3.1 on qₘ, and the assumed properties of the TAC measurement error.

We begin by specifying in detail how we obtain our estimate of the BrAC curve. Independently of qₘ, n TAC observations y_{n,1}, . . . , y_{n,n} are collected from a drinking episode at the increasing times 0≤s_{n,1}< . . . <s_{n,n}≤S, given by

yⱼ=ƒ_{μ}(s_{n, j}; q₀)+ϵ_{n, j} where ƒ_{μ}(s; q)=∫₀^{s}Ce^{A(s-u)}Bμ(u)du (46)

as in (26), where μ is the unknown BrAC curve to be estimated, the matrices A and B depend on q as in (25), and C^{T} is a given fixed vector.

To start, we assume only that the cross ϵ_{n,1}, . . . , ϵ_{n,n} are uncorrelated and have mean zero. We will allow for the possibility that the device used in the field may have different characteristics than the one used for calibration, and for now only impose the condition that the field noise variances are uniformly bounded above by σ̅_̅{̅ƒ̅}̅²̅, some positive constant.

Assume vₙ, n≥1, the empirical probability measure of the TAC observation times, has weak limit v₀. For a given resolution level p≤n we select a basis of p integrable functions {ϕₖ, 1≤k≤p} on [0, S]. The finite basis approximation of μ of order p at time s∈[0, S], with coefficient vector β∈ [symbol] ^{p}, is given by

\hat{μ}(s; β)=ϕ(s)^{T}β where ϕ(s)=[ϕ₁(s), . . . , ϕₚ(s)]^{T}∈ [symbol] ^{p}. (47)

Substitution of μ by the approximation \hat{μ}(s; β) into the integral in (46) yields the predicted TAC values given at ∈[0, S] by

ƒ_{\hat{μ}}(s; q)=(∫₀^{s}Ce^{A(s-u)}Bϕ(u)^{T}du)β;=ψ(s; q)^{T}β where ψ(s; q)∈ [symbol] ^{p}. (48)

Now dropping the double index notation for simplicity, letting sₙ=(s₁, . . , sₙ)^{T}, for a given q and a sequence of symmetric, non-negative definite matrices Mₙ∈R^{p×p}, we take β=β(sₙ, q)∈ [symbol] ^{p} to minimize the objective function

$$J ( β ) = \frac{1}{n} {‖ Y - X ( {s}_{n} ; q ) β ‖}^{2} + {β}^{T} {M}_{n} β = {‖ Z - W ( {s}_{n} ; q ) β ‖}^{2} , \qquad ( 49 )$$

$$where$$

$$\begin{matrix}Y = [ \begin{matrix}{y}_{1} \\ {y}_{2} \\ . \\ . \\ {y}_{n}\end{matrix} ] , & X ( s ; q ) = [ \begin{matrix}{ψ ( {s}_{1} , q )}^{T} \\ ψ {( {s}_{2} , q )}^{T} \\ … \\ … \\ ψ {( {s}_{n} , q )}^{T}\end{matrix} ]\end{matrix} ∈ {ℝ}^{n \times p} ,$$

$$and$$

$${\begin{matrix}Z = {[ \frac{1}{\sqrt{n}} Y , 0 ]}^{T} & and & W ( {s}_{n} ; q ) =\end{matrix} [ \frac{1}{\sqrt{n}} X ( {s}_{n} ; q ) , \sqrt{{M}_{n}} ]}^{T} .$$

By standard results in least squares estimation, when W(sₙ; q)^{T}W(sₙ; q) is full rank, the unique minimizer of J(β) is given by

$$β ( {s}_{n} , q ) = {( {W ( {s}_{n} ; q )}^{T} W ( {s}_{n} ; q ) )}^{- 1} {W ( {s}_{n} ; q )}^{T} Z = ( [ \frac{1}{\sqrt{n}} {X ( {s}_{n} ; q )}^{T} , \sqrt{{M}_{n}} ] [ \begin{matrix}\frac{1}{\sqrt{n}} Y \\ 0\end{matrix} ] = {( \frac{1}{\sqrt{n}} {X ( {s}_{n} ; q )}^{T} X {( {s}_{n} ; q )}^{T} + {M}_{n} )}^{- 1} \frac{1}{n} X {( {s}_{n} ; q )}^{T} Y . \qquad ( 50 )$$

We may also write these equations in a somewhat more convenient form. For n≥1 let

$${G}_{n} ( q ) = {\int }_{0}^{S} ψ ( s ; q ) {ψ ( s ; q )}^{T} {dv}_{n} ( s ) , \qquad ( 51 )$$

$${Z}_{n} ( q ) = {\int }_{0}^{S} ψ ( s ; q ) {f}_{u} ( s ; {q}_{0} ) {dv}_{n} ( s )$$

$$and$$

$${η}_{n} ( q ) = \frac{1}{n} {\sum }_{j = 1}^{n} ψ ( {s}_{j} ; q ) {ϵ}_{j} ,$$

and let these same formulas hold for n=0 upon setting η₀=0. Then, using the alternative notation βₙ(q) for β(sₙ, q), we recover (50) from

βₙ(q)=(Gₙ(q)+Mₙ)⁻¹(Zₙ(q)+ηₙ(q))n≥0, (52)

where, now assuming that the sequence of matrices Mₙ has limit M₀, we also define β₀(q) by (52) applying the stated convention that η₀=0. We note Gₙ(q)+Mₙ will be invertible whenever Mₙ is positive definite. For notational simplicity in what follows, let

Hₙ(q)=Gₙ(q)+Mₙ for n≥0. (53)

When basing inference on the estimate qₘ obtained from a calibration session, as in (47), the estimated BrAC curve is given by μₙ(s; qₘ), where

μₙ(s; q)=ϕ(s)^{T}βₙ(q)n≥0, s∈[0,S]. (54)

Next, define the Lipschitz (semi)norm of a real valued function g with domain [symbol] ⊂ [symbol] by

$${‖ g ‖}_{Lip} = {sup}_{x = y , \{ x , y \} ⊂ 𝒟} \frac{\vert g ( x ) - g ( y ) \vert}{\vert x - y \vert} .$$

In order to control the variation in the estimate βₙ(q) caused by that in qₘ, we introduce the following assumption.

Assumption 3.1 For a given sequence of empirical probability measures vₙ, n≥1 with weak limit v₀, all supported on [0, S], there exist a constant C and a sequence rₙ, n≥1 of real numbers tending to zero as n→∞ such that

$${sup}_{{‖ g ‖}_{Lip} \leq L} \vert {\int }_{0}^{S} g ( u ) {dv}_{n} - {\int }_{0}^{S} g ( u ) {dv}_{0} \vert \leq {LSCr}_{n} \qquad ( 55 )$$

$$for all n \geq 1.$$

As vₙ([0, S])=v₀([0, S]) the difference over which the supremum is taken in (55) is unchanged by replacing g(x) by g(x)+c for any constant c, and hence we may assume that g(0)=0. In particular, for x∈[0,S] we then have that

$$‖ g ‖ = {sup}_{x ∈ [ 0 , S ]} \vert g ( x ) \vert = {sup}_{x ∈ [ 0 , S ]} \vert g ( x ) - g ( 0 ) \vert \leq {sup}_{x ∈ [ 0 , S ]} \vert x \vert {‖ g ‖}_{Lip} = S {‖ g ‖}_{Lip} . \qquad ( 56 )$$

When vₙ is the empirical probability measure of the n equally spaced observations made at times sᵢ=Si/n, i=1, . . . , n, then the limit measure v₀ is the uniform probability measure over [0, S]. Now,

$$\vert {\int }_{0}^{S} g ( u ) {dv}_{n} - {\int }_{0}^{S} g ( u ) {dv}_{0} \vert = \vert \frac{1}{n} {\sum }_{i = 1}^{n} g ( {s}_{i} ) - {\int }_{0}^{S} g ( u ) {dv}_{0} \vert = \vert {\sum }_{i = 1}^{n - 1} ( {\int }_{{s}_{i}}^{{s}_{i + 1}} ( g ( {s}_{i} ) - g ( u ) ) {dv}_{0} ) + \frac{g ( S )}{n} - {\int }_{0}^{{s}_{1}} g ( u ) du \vert \leq {‖ g ‖}_{Lip} {\sum }_{i = 1}^{n - 1} {\int }_{{s}_{i}}^{{s}_{i + 1}} \vert {s}_{i} - u \vert du + \frac{S {‖ g ‖}_{Lip}}{n} + \frac{{S}^{2} {‖ g ‖}_{Lip}}{2 {n}^{2}} = \frac{( n - 1 ) {S}^{2} {‖ g ‖}_{Lip}}{2 {n}^{2}} + \frac{S {‖ g ‖}_{Lip}}{n} + \frac{{S}^{2} {‖ g ‖}_{Lip}}{2 {n}^{2}} \leq C {‖ g ‖}_{Lip} {r}_{n} ,$$

and Assumption 3.1 holds with C=S²+S, and rₙ=1/n.

Alternatively, when vₙ is the empirical measure of times X₁, . . . , Xₙ, independent with common distribution v₀ supported on [0, S], then Assumption 3.1 holds with rₙ=1/\sqrt{n} with high probability. In particular, there exists a constant C such that

$$E [ {W}_{n} ] \leq CLS where {W}_{n} = {sup}_{{‖ g ‖}_{Lip} \leq L} \sqrt{n} \vert {\int }_{0}^{S} g ( u ) {dv}_{n} - {\int }_{0}^{S} g ( u ) {dv}_{0} \vert . \qquad ( 57 )$$

For Sₙ=\sqrt{n}Wₙ, we have

P(Sₙ≥E[Sₙ]+\sqrt{(4∥g∥E[Sₙ]+2n∥g∥²)x}+∥g∥x/3)≤e^{-x} for all x≥0.

Using the bound (57), and recalling (56), with C being a constant not necessarily the same at each occurrence, we obtain

E[Sₙ]+{square root over (4∥g∥E[Sₙ]+2n∥g∥²)x)}+∥g∥x/3 ≤LS(C\sqrt{n}+\sqrt{(C\sqrt{n}+2n)x}+x/3)≤LSC(\sqrt{n}+\sqrt{nx}+x),

implying that, with rₙ=1/\sqrt{n}

$$P ( \frac{{W}_{n}}{\sqrt{n}} \geq LSC ( 1 + \sqrt{x} + x ) {r}_{n} ) \leq P ( \frac{{W}_{n}}{\sqrt{n}} \geq LSC ( 1 + \sqrt{x} + x / \sqrt{n} ) {r}_{n} ) \leq {e}^{- x} .$$

Hence, given α∈(0,1), inequality (55) in Assumption 3.1 holds with probability at least 1−α for some constant C depending only on α and rₙ=1/\sqrt{n}.

We next pause to prove some technical results that will be invoked in Theorems 3.4 and 3.5; the partials inside the integral in (58) can be computed applying (39) of Lemma 3.3.

Lemma 3.4 The partial derivatives of ψ(s, q) in (48) with respect to the i^{th} component of q for i=1,2 exist and are given by

∂ᵢψ(s, q)^{T}=∫₀^{s}C(∂ᵢe^{A(s-u)}B)ϕ(u)^{T}du, (58)

are bounded and continuous as a function of s∈[0,S] and continuous in q on [symbol] as given in (24), and there exists a finite constant L such that on [0, S]

$${sup}_{q ∈ 𝒞} ‖ ψ ( \cdot ; q ) ‖ < ∞ and {sup}_{q ∈ 𝒞} {‖ ψ ( \cdot ; q ) ‖}_{Lip} \leq L . \qquad ( 59 )$$

Further, at any q∈ [symbol] . Gₙ(q) and Zₙ(q) given in (51) are continuous and

∂ᵢZ₀(q)=∫₀^{S}∂ᵢψ(s, q)ƒ_{μ}(s, q₀)dv₀ and

∂ᵢG₀(q)=∫₀^{S}(∂ᵢψ(s, q)ψ(s, q)^{T}+ψ(s, q)∂ᵢψ(s, q)^{T})dv₀ (60)

exist and are continuous. For β in (52), the partials

∂ᵢβ₀(q)=H₀(q)⁻¹∂ᵢZ₀(q)−H₀(q)⁻¹(∂ᵢG₀)H₀(q)⁻¹Z₀(q) (61)

exist and are continuous at any q∈ [symbol] for which H₀(q)⁻¹ exists.

Proof: The claims for ψ(s, q) and its partial derivatives follow directly from Lemma 3.3, and the integrability of ϕₖ(u), k=1, . . . , p on [0,S]. The claims on the partials of Gₙ(q) and Zₙ(q), that imply the continuity of these functions, follow from the continuity of ƒ_{μ}(s, q₀) over s∈[0, S] as provided by Lemma 3.3, the demonstrated properties of ψ(s, q) and the dominated convergence theorem. The well known formula for differentiating matrix inverses yields (61) and the final claim, noting that the map taking a matrix to its inverse is continuous.

Lemma 3.5 Let Gₙ(q) and Zₙ(q) be as in (51) for n≥0. and suppose H₀(q)⁻¹ exists for some q₀∈ [symbol] . Then there exists a compact set [symbol] ⊂ [symbol] with non-empty interior containing q₀ such that H₀(q)⁻¹ exists for all q∈ [symbol] , and if Assumption 3.1 holds then there exists a constant C such that for all n sufficiently large

$${sup}_{q ∈ 𝒞} ‖ {G}_{n} ( q ) - {G}_{0} ( q ) ‖ \leq {Cr}_{n} , {sup}_{q ∈ ??} ‖ {{H}_{n} ( q )}^{- 1} - {{H}_{0} ( q )}^{- 1} ‖ \leq {Cr}_{n} \qquad ( 62 )$$

$$and {sup}_{q ∈ 𝒞} ‖ {Z}_{n} ( q ) - {Z}_{0} ( q ) ‖ \leq {Cr}_{n} ,$$

and for all n sufficiently large and n=0.

$${sup}_{q ∈ 𝒞} ‖ {{H}_{n} ( q )}^{- 1} ‖ < ∞ and {sup}_{q ∈ 𝒞} ‖ {Z}_{n} ( q ) ‖ < ∞ . \qquad ( 63 )$$

When the field error variables ϵ₁, . . . , ϵₙ have mean zero, and are uncorrelated with variances uniformly dominated by σ̅_̅{̅ƒ̅}̅²̅, then

$${sup}_{q ∈ 𝒞} ‖ Var ( {η}_{n} ( q ) ) ‖ \leq {\overline{σ}}_{f}^{2} {sup}_{s ∈ [ 0 , S ] , q ∈ 𝒞} {‖ ψ ( s , q ) ‖}^{2} / n . \qquad ( 64 )$$

Proof: Denoting the i^{th} largest eigenvalue of a symmetric matrix by λᵢ(⋅), by Weyl's theorem (see Theorem 4.3.1 of [Horn and Johnson, 2012]), for N₁ and N₂ symmetric matrices in [symbol] ^{p×p},

|λᵢ(N₁)−λᵢ(N₂))|≤∥N₁−N₂∥ for i=1, . . . , p. (65)

The matrix G₀(q) is continuous in q by Lemma 3.4, hence H₀(q) is likewise continuous. As G₀(q) and M₀ are non-negative definite for all q and H₀(q) is invertible at q₀ by assumption, the continuity of λ₁(⋅) provided by (65) yields the existence of ϵ>0 such that λ₁(H₀(q))>ϵ for all q in some bounded open subset of [symbol] containing q₀, and again by continuity this same inequality holds in the non-strict sense over the closure; the first claim in (63) follows.

By Lemma 3.4 and Assumption 3.1

$${sup}_{q ∈ 𝒞} ‖ {G}_{n} ( q ) - {G}_{0} ( q ) ‖ \leq {CLr}_{n} , \qquad ( 66 )$$

for some constant C, thus proving the first claim of (62), and the final claim of (64). Since rₙ→0 as n→∞, for all n sufficiently large CLrₙ<ϵ/2, implying, by (65), that in [symbol] λ₁(Hₙ(q))>ϵ/2. Hence, for such n,

$${sup}_{q ∈ 𝒞} ‖ {{H}_{n} ( q )}^{- 1} - {{H}_{0} ( q )}^{- 1} ‖ = {sup}_{q ∈ 𝒞} \vert {{λ}_{c} ( {H}_{n} ( q ) )}^{- 1} - {{λ}_{1} ( {H}_{0} ( q ) )}^{- 1} \vert = {sup}_{q ∈ 𝒞} \frac{{λ}_{1} ( {H}_{n} ( q ) ) - {λ}_{1} ( {H}_{0} ( q ) )}{{λ}_{1} ( {H}_{n} ( q ) ) {λ}_{1} ( {H}_{0} ( q ) )} \vert < \frac{2}{{ϵ}^{2}} {sup}_{q ∈ 𝒞} \vert {λ}_{1} ( {H}_{n} ( q ) ) - {λ}_{1} ( {H}_{0} ( q ) ) \vert \leq \frac{2}{{ϵ}^{2}} {sup}_{q ∈ 𝒞} ‖ {G}_{n} ( q ) - {G}_{0} ( q ) ‖ \leq \frac{{CLr}_{n}}{{ϵ}^{2}} ,$$

where the penultimate inequality follows from (65) and by noting that Hₙ−H₀=Gₙ−G₀. and the final inequality from (66). The proof of the the second claim in (62) is complete.

As the first claim in (63) holds for n=0, it holds for all n sufficiently large by the triangle inequality and the first claim in (62). Arguing as for the first claim in (62) and using the smoothness and continuity properties of ƒ_{μ}(s, q₀) provided by Lemma 3.3, the second follows similarly as a consequence of Assumption 3.1; the second claim of (63) follows by the triangle inequality, as did the first. The final claim (64) follows directly from the definition (51) and the stated assumptions on the error terms.

Moving to the properties of βₙ(qₘ), note the decomposition

βₙ(qₘ)−β₀(q₀)=(βₙ(qₘ)−β₀(qₘ))+(β₀(qₘ)−β₀(q₀)), (67)

and for the first term, applying (52) we may write

$${β}_{n} ( {q}_{m} ) - {β}_{0} ( {q}_{m} ) = ( {{H}_{n} ( {q}_{m} )}^{- 1} - {{H}_{0} ( {q}_{m} )}^{- 1} ) {Z}_{n} ( {q}_{m} ) + {{H}_{0} ( {q}_{m} )}^{- 1} ( {Z}_{n} ( {q}_{m} ) - {Z}_{0} ( {q}_{m} ) ) + {{H}_{n} ( {q}_{m} )}^{- 1} {η}_{n} ( {q}_{m} ) . \qquad ( 68 )$$

We prove the following distributional limit theorem for the final term in (68).

Lemma 3.6 Assume H₀(q)⁻¹ exists for q₀∈ [symbol] . In addition, let the errors ϵᵢi≥1 be independent, mean zero with common variance of σ_{ƒ}²∈(0, ∞), and suppose for some η>0 and K>0 that E|ϵᵢ|^{2+η}≤K for all i≥1. If \sqrt{m}(qₘ−q₀)=Oₚ(1). Assumption 3.1 holds. and that n, m→∞ so that \sqrt{m}rₙ→0, then

\sqrt{m}Hₙ(qₘ)⁻¹ηₙ(qₘ)=\sqrt{m}H₀(q₀)⁻¹ηₙ(q₀)+oₚ(1) (69)

and

\sqrt{n}Hₙ(qₘ)⁻¹ηₙ(qₘ)→_{d}Y˜ [symbol] (0, σ_{ƒ}²H₀(q₀)⁻¹G₀(q₀)H₀(q₀)⁻¹). (70)

The condition that \sqrt{m}(qₘ−q₀)=Oₚ(1) is implied by the conditions of Theorem 3.1, as they provide the stronger conclusion that this quantity converges in distribution.

Proof: By the consistency of qₘ for q₀, we may assume without loss of generality that qₘ is contained in [symbol] given in Lemma 3.5. Writing

magentaHₙ(qₘ)⁻¹ηₙ(qₘ)

magenta=(Hₙ(qₘ)⁻¹−H₀(qₘ)⁻¹)ηₙ(qₘ)+H₀(qₘ)⁻¹(ηₙ(qₘ)−ηₙ(q₀))

magenta+(H₀(qₘ)⁻¹−H₀(q₀)⁻¹)ηₙ(q₀)+H₀(q₀)⁻¹ηₙ(q₀), (71)

we show (69) by demonstrating that the first three terms tend to zero in probability after scaling by \sqrt{m}. We see the claim is true for the first term by virtue of the second inequality of (62) and (64) of Lemma 3.5.

For the second term, define

$${A}_{n , m} = \sqrt{m} ( {η}_{n} ( {q}_{m} ) - {η}_{n} ( {q}_{0} ) ) = \frac{\sqrt{m}}{n} {\sum }_{j = 1}^{n} ( ψ ( {s}_{j} , {q}_{m} ) - ψ ( {s}_{j} , {q}_{0} ) ) {ϵ}_{j} .$$

Let δ>0 be given and [symbol] be as in Lemma 3.5. Since \sqrt{m}(qₘ−q₀)=Oₚ(1), there exists M such that

$${limin}_{m} fP ( {Ω}_{M , m} ) \geq 1 - δ where {Ω}_{M , m} = \{ \sqrt{m} ‖ {q}_{m} - {q}_{0} ‖ \leq M \} .$$

By the independence between qₘ and ϵⱼ, j=1, . . . , n,

E(A_{n,m}1_{Ω}_{_{M,m}}|qₘ)=0. (72)

Hence, via the conditional variance formula, and that Ω_{M,m} is measurable with respect to qₘ, we obtain

$$Var ( {A}_{n , m} {1}_{{Ω}_{M , m}} ) = E ( Var ( {A}_{n , m} {1}_{{Ω}_{M , m}} \vert {q}_{m} ) ) = \frac{m {σ}_{f}^{2}}{{n}^{2}} E [ {\sum }_{j = 1}^{n} {( ψ ( {s}_{j} , {q}_{m} ) - ψ ( {s}_{j} , {q}_{0} ) )}^{2} {1}_{{Ω}_{M , m}} ] \leq \frac{{σ}_{f}^{2} {LC}_{ψ}^{2}}{n} E [ m ‖ {q}_{m} - {q}_{0} {‖}^{2} {1}_{{Ω}_{M , m}} ] \leq \frac{{σ}_{f}^{2} {LC}_{ψ}^{2}}{n} {M}^{2} \to 0 as n \to ∞ ,$$

where we used Lemma 3.4 in the first inequality. Hence, now invoking (72) and the δ>0 is arbitrary, the second term is oₚ(1) via (63).

For the third term we use the consistency of qₘ for q₀, the continuity of H₀(q) in q, and in addition (64). The proof of (69) is complete.

By (69) it suffices to show that

$$\sqrt{n} {η}_{n} ( {q}_{0} ) = {\sum }_{i = 1}^{n} {X}_{n , i} {\to }_{d} 𝒩 ( 0 , {σ}_{f}^{2} {G}_{0} ( {q}_{0} ) ) where {X}_{n , i} = \frac{1}{\sqrt{n}} ψ ( {s}_{i} ; {q}_{0} ) {ϵ}_{i} .$$

We apply Lemma 2.1, noting that the first condition in (17), that Gₘ(q₀) converges to G₀(q₀), holds by Lemma 3.5.

It remains to verify the second condition in (17). The vector ψ(s, q) is uniformly bounded over s∈[0, S] and q∈ [symbol] by (59) of Lemma 3.4. Hence, for some constant C, as n→∞,

$${{\sum }_{i = 1}^{n} 𝔼 {‖ {X}_{n , i} ‖}^{2 + η} \leq \frac{1}{{n}^{2 + η / 2}} {\sum }_{i = 1}^{n} ‖ ψ ( {s}_{i} ; {q}_{0} ) ‖}^{2 + η} E {\vert {ϵ}_{i} \vert}^{2 + η} \leq {n}^{- η / 2} {C}^{2 + η} {K}^{4 + 2 η} \to 0 ,$$

thus completing the proof of the lemma.

We now prove a consistency result for βₙ(qₘ), and apply it to show that the BrAC curve estimate converges uniformly in probability to μ₀(s; q₀), the unique function in the L² space spanned by the first p basis elements that is closest to the true BrAC curve.

Theorem 3.4 Suppose that qₘ is consistent for q₀ as m→∞, that H₀(q₀) is invertible, and that the error variables ϵ₁, . . . , ϵₙ have mean zero, and are uncorrelated with variances dominated by σ̅_̅{̅ƒ̅}̅². In addition, let Assumption 3.1 hold with some sequence rₙ tending to zero. Then

βₙ(qₘ)→ₚβ₀(q₀),

and the reconstructed BrAC curve obeys ∥μₙ(⋅; qₘ)−μ₀(⋅; q₀)∥→ₚ0.

Proof: Let [symbol] ⊂ [symbol] be given by Lemma 3.5. By the consistency of qₘ, for any given δ there exists m₀ such that for all m≥m₀ the probability of Eₘ={qₘ∈ [symbol] } is at least 1−δ. By the triangle inequality, to show βₙ(qₘ) is consistent, it suffices to verify that the two terms on the right side of (67), with q=qₘ, both tend to zero in probability. The first term converges to zero in probability on Eₘ by virtue of (68), Lemma 3.5 and Assumption 3.1. The second term tends to zero by virtue of the consistency of qₘ and the continuity of the function β₀(⋅).

The last claim follows from the first, using that ϕ(s) is bounded on [0, S], and from (47), yielding

$${sup}_{s ϵ [ 0 , S ]} \vert {μ}_{n} ( s ; {q}_{m} ) - {μ}_{0} ( s ; {q}_{0} \vert \leq ‖ ϕ ‖ ‖ {β}_{n} ( q ) - {β}_{0} ( {q}_{0} ) ‖ .$$

Lastly, we determine the asymptotic distribution of the estimated BrAC curve, properly scaled, and show in (75) how uniform confidence bounds can be constructed asymptotically; an expression for the partials required for the computation of K in (73) is provided by (61) and (60).

Theorem 3.5 Suppose that

\sqrt{m}(qₘ−q₀)→_{d} [symbol] (0, σ²Γ⁻¹)asm→∞

for some invertible matrix Γ, that G₀(q₀) is invertible, that Assumption 3.1 holds for rₙ \sqrt{m}rₙ→0, that ϵᵢ, i=1, . . . , n are independent mean zero random variables with variance σ_{ƒ}² and uniformly bounded 2+η moments for some η>0 and that sup_{k≥1}∥ϕₖ∥<∞.

If m/n→ρ∈[0, ∞),

\sqrt{m}(βₙ(qₘ)−β₀(q₀))→_{d} [symbol] (0, σ²K^{T}Γ⁻¹K+ρσ_{ƒ}²G₀⁻¹(q₀))

where K=∂_{q}β₀(q₀)^{T}, (73)

and

Wₘ(s)=\sqrt{m}(μₙ(s; qₘ)−μ₀(s; q₀))→_{d}W_{σ, ρ}(s)

where W_{σ, ρ}(s)=ϕ(s)^{T}(σK^{T}Γ^{-1/2}Z₁+\sqrt{ρ}σ_{ƒ}G₀^{-1/2}(q₀)Z₂) (74)

as processes on the space C[0, S] of continuous functions on [0, S], endowed with the supremum norm, where the Γ^{-1/2} and G₀^{-1/2}(q₀) are the the unique positive definite square roots of Γ⁻¹ and G₀⁻¹(q₀) respectively, and Z₁˜ [symbol] (0, I) and Z₂˜ [symbol] (0, I), are standard two dimensional Gaussian random vectors. In addition,

$${sup}_{s ∈ [ 0 , S ]} \vert \sqrt{m} ( {μ}_{n} ( s ; {q}_{m} ) - {μ}_{0} ( s ; {q}_{0} ) ) \vert {\to }_{d} {sup}_{s ∈ [ 0 , S ]} \vert {W}_{σ , ρ} ( s ) \vert . \qquad ( 75 )$$

If m/n→ρ=∞, then (73), (74) and (75) hold with the scaling \sqrt{m} replaced by \sqrt{n} and the parameters of the limiting distributions in those displays set to (σ, ρ)=(0,1).

Remark 3.1 The boundary case ρ=0 corresponds to the situation where the number of observations taken in the field is so large that the variability of the resulting BrAC estimate depends only on the uncertainty in the parameter estimate q₀. hence asymptotically equivalent to the situation where the field observations are taken without noise. At the other extreme, the case ρ=∞ reflects the situation where the number of observations taken in the calibration experiment in the lab is so large that for the purposes of BrAC estimation, the parameter q₀ is, in a practical sense, known.

Proof: By the delta method using that ∂_{q}β(q) is continuous in a neighborhood of q₀ by Lemma 3.4, w obtain

\sqrt{m}(β₀(qₘ)−β₀(q₀))→_{d}σU˜ [symbol] (0,σ²K^{T}Γ⁻¹K). (76)

Next we note that by the triangle inequality the first two terms in (68) tend to zero by the consistency of qₘ for q₀, Lemma 3.5 and the condition \sqrt{m}rₙ→0. Now suppose that m/n→ρ∈[0, ∞) by Lemma 3.6, and adopting the notation in (70).

$$\sqrt{m} {{G}_{n} ( {q}_{m} )}^{- 1} {η}_{n} ( {q}_{m} ) = \sqrt{\frac{m}{n}} ( \sqrt{n} {{G}_{n} ( {q}_{m} )}^{- 1} {η}_{n} ( {q}_{m} ) ) {\to }_{d} \sqrt{ρ} Y . \qquad ( 77 )$$

Using (69) of Lemma 3.6 we see that Y is the distributional limit of a quantity not depending on qₘ, plus a term that tends to zero in probability, thus showing that U and Y are asymptotically independent. Hence

\sqrt{m}(βₙ(qₘ)−β₀(q₀))→_{d}σU+\sqrt{ρ}Y, (78)

completing the proof of (73)

Letting α(n, m)=\sqrt{m}(βₙ(qₘ)−β₀(q₀)), by the definition of Wₘ in (74), μₙ in (54), and the convergence in (78), for d≥1 the finite dimensional distributions of Wₘ at the times points 0≤s₁< . . . <s_{d}≤S converge to those of W₀, as

[Wₘ(s₁), . . . , Wₘ(s_{d})]^{T}=[ϕ(s₁), . . . , ϕ(s_{d})]^{T}α(n, m) →_{d}[ϕ(s₁), . . . , ϕ(s_{d})]^{T}(σU+\sqrt{ρ}Y)=_{d}[W_{σ, ρ}(s₁), . . . , W_{σ, ρ}(s_{d})]^{T}. (79)

Define the modulus of continuity of a continuous function ϕ(s) on [0, S] by

$${Ω}_{ϕ} ( δ ) = {sup}_{\vert s - t \vert < δ , 0 \leq s , t \leq S} \vert ϕ ( s ) - ϕ ( t ) \vert for 0 < δ \leq S .$$

The proof will be complete upon showing following two properties that together imply {Wₘ, m≥1} is tight: for every positive η, there exists a such that

P(|Wₘ(0)|>a)≤η for all m≥1, (80)

and for every positive η and ϵ, there exists δ>0 and an integer m₀ such that

P(Ω_{W}_{ₘ}(δ)≥ϵ)≤η for all m≥m₀. (81)

Condition (80) follows from (79) with d=1 and s₁=0; as Wₘ(0) converges in distribution, the sequence Wₘ(0) is tight.

Let positive η and ϵ be given. As α(n, m) converges in distribution there exists C such that P(∥α(n, m)∥≤C)≥1−η for all m≥1. Let

δ=inf{τ:Ω_{ϕ}_{ₖ}(τ)<ϵ/Cp, 1≤k≤p};

this quantity will be positive for all ϵ>0 as each basis function ϕₖ, k=1,2, . . . , p is continuous on [0, S], and therefore uniformly continuous. Thus, with probability at least 1−η, for |s−t|<δ, 0≤s, t≤S and all m≥1,

|Wₘ(s)−Wₘ(t)|=|(ϕ(s)−ϕ(t))^{T}α(n, m)|≤∥ϕ(s)−ϕ(t)∥∥α(n, m)∥≤C∥ϕ(s)−ϕ(t)∥≤CΣᵢ₌ₖ^{p}|ϕₖ(s)−ϕₖ(t)|≤CΣᵢ₌ₖ^{p}Ω_{ϕ}_{ₖ}(δ)≤ϵ,

from which (81) follows with m₀=1.

Lastly, consider the case m/n→∞. Scaling now by \sqrt{n} rather than \sqrt{m}, we see that

$$\sqrt{n} ( {β}_{n} ( {q}_{m} ) - {β}_{0} ( {q}_{0} ) ) = \sqrt{\frac{n}{m}} ( \sqrt{m} ( {β}_{n} ( {q}_{m} ) - {β}_{0} ( {q}_{0} ) ) ) {\to }_{p} 0 ,$$

as the first term tends to zero and the second one converges in distribution. Hence, the only term contributing is (70), and the argument for the previous case carries through with essentially no modification.

As K and Γ in (75) depend on the unknown q₀, in practice these quantities can be estimated by their values at along a sequence of consistent estimates qₘ, m≥1. As K and Γ are continuous at q₀, these resulting estimates will likewise be consistent. Similar remarks apply as to the estimation of σ_{ƒ}² and G₀(q₀).

Remark 3.2 The regularization matrix Mₙ in the objective function (49) is used to avoid numerical instability; details on the relevant choice of Mₙ used here can be found in Section 4. However, regularization can induce bias. To illustrate, assume for some p that μ(s)∈span{ϕ₁(s), . . . , ϕₚ(s)}, that is,

μ(s)=ϕ(s)^{T}β_{*} for some β_{*}∈ [symbol] ^{p}. (82)

In the limiting case n=0 of (52) for q=q₀, in light of (47), (48) and (82), we obtain B₀(q₀)=(G₀(q₀)+M₀)⁻¹Z₀(q₀)=(G₀(q₀)+M₀)⁻¹G₀(q₀)β_{*}. In particular, the limiting coefficient vector β₀(q₀) μ be biased for the true β₈ unless M₀=0.

Regularization Details (Section 4). For u in the span of a basis {ϕᵢ, i=0, . . . , p} of differentiable functions on [0, T], there exists a unique vector β=[β₀, . . . , βₚ]^{T}∈ [symbol] ^{p+1} such that

$$μ ( t ) = {\sum }_{i = 0}^{p} {β}_{i} {ϕ}_{i} ( t ) = {Φ ( t )}^{T} β and hence {μ}^{′} ( t ) = {\sum }_{i = 0}^{p} {β}_{i} {ϕ}_{i} , ( t ) = {{Φ}^{′} ( t )}^{T} β$$

where ϕ(t)=[ϕ₀(t), . . . , ϕₚ(t)]^{T}∈ [symbol] ^{p+1}. In this case, we the express the L² norm of μ and its derivative as a quadratic form involving matrices R and S given by

∫₀^{T}μ²(t)dt=∫₀^{T}(ϕ(t)^{T}β)^{T}ϕ(t)^{T}βdt=β^{T}[∫₀^{T}ϕ(t)ϕ(t)^{T}dt]β=:β^{T}Rβ,

and

∫₀^{T}[μ′(t)]²dt=∫₀^{T}(ϕ′(t)^{T}β)^{T}ϕ′(t)^{T}βdt=β^{T}[∫₀^{T}ϕ′(t)ϕ′(t)^{T}dt]β=:β^{T}Sβ,

We regularize using a linear combination of these penalty terms, as

M=λ∫₀^{T}μ²(t)dt+μ∫₀^{T}[μ′(t)]²dt=λR+μS.

We specialize now to the chapeau functions given by

$${ϕ}_{j} ( t ) = \{ \begin{matrix}( p / T ) t - ( j - 1 ) & t ∈ [ ( j - 1 ) T / p , jT / p ] \\ 2 - ( ( p / T ) t - ( j - 1 ) ) & t ∈ [ jT / p , ( j + 1 ) T / p ] \\ 0 & otherwise ,\end{matrix} \qquad ( 83 )$$

and letting r=T/p, we claim

$${R}_{ij} = 〈 {ϕ}_{i} , {ϕ}_{j} 〉 = \{ \begin{matrix}r / 6 ( {1}_{j \geq 1} + {1}_{j \leq n - 1} ) & i = j \\ r / 6 & \vert i = j \vert = 1 \\ 0 & \vert i - j \vert \geq 2 ,\end{matrix}$$

$$and$$

$${S}_{ij} = 〈 {ϕ}_{{i}^{′}} , {ϕ}_{{j}^{′}} 〉 = \{ \begin{matrix}{r}^{- 1} ( {1}_{j \geq 1} + {1}_{j \leq n - 1} ) & i = j \\ - {r}^{- 1} & \vert i = j \vert = 1 \\ 0 & \vert i - j \vert \geq 2.\end{matrix}$$

We note that the result of zero when |i−j|≥2 follows immediately from the fact that ϕᵢ and ϕⱼ have disjoint support in this case.

Defining

ψ₋(t)=(1+t)1_{[-1,0]} and ψ₊(t)=(1−t)1_{[0,1]},

we may write

ϕⱼ(t)=ψ₋(t/r−j)1_{j≥1}+ψ₊(t/r−j)1_{j≤p-1}.

We note that

∫₀¹ψ₊²(t)dt=∫₀¹ψ₋²(t)dt=⅓

implying ∫₀^{T}ψ₊²(t/r−j)dt=∫₀^{T}ψ₋²(t/r−j)dt=r/3, (84)

and that

∫₀¹ψ₋(t−1)ψ₊(t)dt=∫₀¹t(1−t)dt=⅙

implying ∫₀^{T}ψ₋(t/r−(j−1))ψ₊(t/r−j)dt=r/6. (85)

Further, as ψ_{-′}(t)=1_{[0,1]} and ψ_{+′}(t)=−1_{[0,1]}, we have

ψ_{j′}(t)=r⁻¹(1_{[(j-1)r,jr]}1_{j≥1}−1_{[jr,(j+1)r]}1_{j≤p-1}). (86)

For i=j, by (84) we obtain

[symbol]

ϕᵢ, ϕᵢ [symbol] =∫₀^{T}ϕᵢ²(t)dt

$$= {\int }_{0}^{T} ψ - {( t / r - j )}^{2} {1}_{j \geq 1} dt + {\int }_{0}^{T} {ψ}_{+} {( t / r - j )}^{2} {1}_{j \leq p - 1} dt$$

$$= \frac{r}{3} {1}_{j \geq 1} + \frac{r}{3} {1}_{j \leq p - 1} .$$

For the remaining case |i−j|=1, by relabelling we may assume i=j−1, j=1, . . . , p. In this case, by (85) we have

[symbol]

ϕⱼ₋₁, ϕⱼ [symbol] =∫₀^{T}ϕⱼ₋₁(t)ϕⱼ(t)dt=∫₀^{T}ψ₋₁(t/r−(j−1))ψ₊(t/r−j)dt=r/6.

Likewise, using (86),

[symbol]

ϕ_{i′}, ϕ_{i′} [symbol] =∫₀^{T}[ϕ_{i′}(t)]²dt=r⁻²∫₀^{T}(1_{[(j-1)r,jr]}1_{j≥1}+1_{[jr,(j+1)r]}1_{j≥p . . . 1})dt=r⁻¹(h1_{j≥1}+1_{j≤p-1})

and for j=1, . . . , p, noting that

(1_{[(j-2)r,(j-1)r]}1_{j≥2}−1_{[(j-1)r,jr]}1_{j≤p})×(1_{[(j-1)r,jr]}1_{j≥1}−1_{[jr,(j+1)r]}1_{j≤p-1})=−1_{[(j-1)r,jr]}1_{1≤j≤p},

we obtain

[symbol]

ϕⱼ₋₁′, ϕ_{j′} [symbol] =−r⁻²∫₀^{T}1_{[(j-1)r,jr]}1_{i≤j≤p}dt=−r⁻¹1_{1≤j≤p}

### Transdermal Blood Alcohol Monitoring: Simulations and Data Analysis (Section 5)

In both the simulation and real data study presented below we investigate the case where data are collected from a single drinking episode. The computations were carried out in MATLAB and the optimization producing the estimate of the parameter q was solved using the Optimization Toolbox routine FMINCON.

Simulation Studies (Section 5.1) Firstly, our simulation study aims to validate our theoretical results on the consistency and asymptotic normality of the parameter estimate given in Theorem 3.1, and to also illustrate the practical impact of the number of observations on its behavior.

To reflect a simple real-world situation, BrAC was simulated using a small but realistic drinking diary that consists of a single drink 6 minutes after the beginning of the drinking session. BrAC was computed using the Michaelis-Menten approach that models the metabolic effects of the ethanol specific enzymes ADH and ALDH typically found in the liver, and also known to be present in trace amounts in the skin.

For simplicity, we set q₀=(1,1) to be the true value of the parameter q and T=1 hour to be the duration of the drinking session. Also for simplicity we consider the following choice of vectors and matrices in (4) and (5), D=I₂, E=O₂, C=(1,0) and F=(1,0)^{T}. Then, equally spaced TAC measurement were calculated after adding independent error terms each distributed as [symbol] (0, σ²) with σ=0.01 to the expression given by (26).

Calculating the theoretical limiting covariance matrix in Theorem 3.1 we obtain

$$\sum = ( \begin{matrix}16.4404 & - 7.2947 \\ - 7.2947 & 3.4586\end{matrix} ) .$$

A comparison between Σ and the scaled sample covariance matrices of \hat{q} is shown in Table 1, validating the theoretical results, and showing that, for the current set of parameters, 60 observations gives a reasonably close estimate to the true values.

TABLE 1

| Number of | | |
|---|---|---|
| TAC | Mean Parameter | Scaled Sample |
| observations | Estimate | Covariance Matrix |
| 20 | $( \begin{matrix}0.9447 \pm 0.1549 \\ 1.0597 \pm 0.0684\end{matrix} )$ | $( \begin{matrix}12.6231 & - 5.2525 \\ - 5.2525 & 2.4586\end{matrix} )$ |
| 60 | $( \begin{matrix}1.0375 \pm 0.0997 \\ 1.0042 \pm 0.0435\end{matrix} )$ | $( \begin{matrix}15.679 & - 6.6024 \\ - 6.6024 & 2.9912\end{matrix} )$ |
| 100 | $( \begin{matrix}0.9762 \pm 0.0805 \\ 1.0228 \pm 0.0381\end{matrix} )$ | $( \begin{matrix}17.0397 & - 7.826 \\ - 7.826 & 3.8215\end{matrix} )$ |

(Sample mean and covariance matrices of samples that consist of 100 \hat{q} estimators)

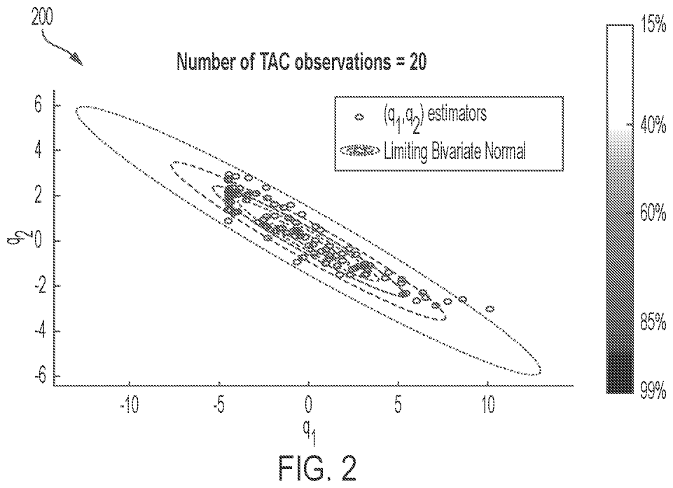

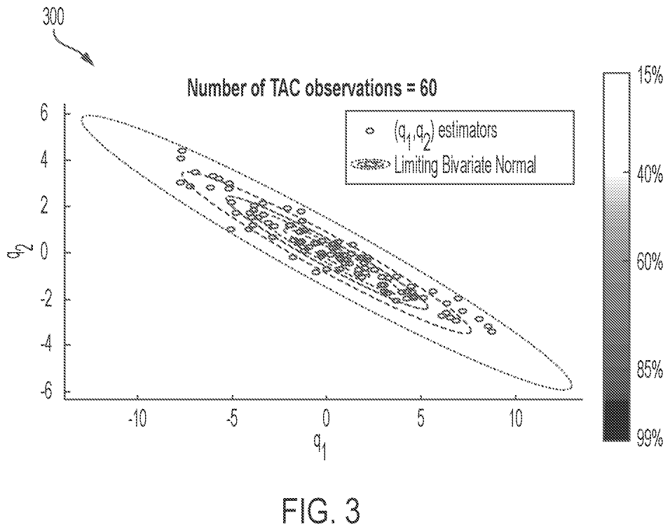

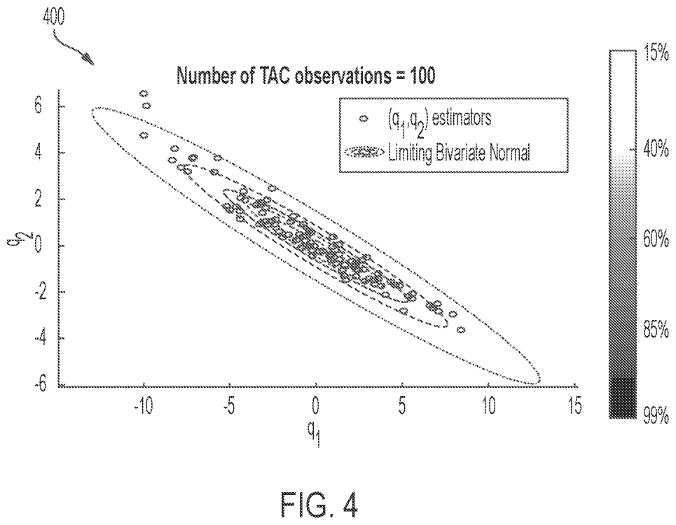

FIGS. 2, 3, and 4 show the values of the \hat{q} estimators calculated from the simulated data for 20 (FIG. 2, 200), 60 (FIGS. 3, 300) and 100 (FIG. 4, 400) observations, respectively, along with levels curves of the limiting bivariate normal distribution in Theorem 3.1. FIG. 2 illustrates values 200 of the \hat{q} estimators obtained when using 20 TAC observations over T=1 hour. FIG. 3 illustrates values 300 of the \hat{q} estimators obtained when using 60 TAC observations over T=1 hour. FIG. 4 illustrates values 400 of the \hat{q} estimators obtained when using 100 TAC observations over T=1 hour.

In a second experiment, our simulation study aims to validate the results of Theorem 3.5 and more specifically to provide confidence bounds for the reconstructed BrAC curve using the result in (75). We use μ(s; β)=−0.2s(s−1) to generate the true BrAC curve and choose the orthonormal polynomial basis ϕ(s)=[\sqrt{3}s, \sqrt{80}(s²−0.75s)]^{T}∈ [symbol] ². Further, according to Theorem 3.5 we let qₘ to be generated from [symbol] (0, σ²Γ⁻¹) where for simplicity we take σ²=1 and Γ to be the identity matrix.

The running time of these experiments may be long due to the computation in (4) of the matrix exponential of A, which in general is not symmetric. For that reason, its worth noting that speed can be improved using the following diagonalization procedure. Letting ϕᵢ, i=1, . . . , n be the basis for the finite dimensional approximation discussed in xxx, define matrices K₁, K₂ and M by

K_{1,ij}=∫₀¹ϕ_{i′}(u)ϕ_{j′}(u)du, K_{2,ij}=ϕᵢ(0)ϕⱼ(0) and Mᵢⱼ=∫₀¹ϕᵢ(u)ϕⱼ(u)du.

### Then

D=−M⁻¹K₁, E=−M⁻¹K₂ and A=q₁D+E=−M⁻¹(q₁K₁+K₂). (87)

We note that the matrices M and q₁D+E are symmetric. Multiplying the final expression for M in (87) on the left and right by M^{1/2} and M^{-1/2} respectively we obtain

M^{1/2}AM^{-1/2}=−M^{-1/2}(q₁K₁+K₂)M^{-1/2}. (88)

Note that the right hand side of (88) is symmetric and therefore we may apply the spectral theorem and write

M^{1/2}AM^{-1/2}=S_{q}_{₁}L_{q}_{₁}S_{q}_{₁}⁻¹ and so, for t∈ [symbol] M^{1/2}tAM^{-1/2}=S_{q}_{₁}tL_{q}_{₁}S_{q}_{₁}⁻¹ (89)

where S_{q}_{₁} is invertible and L_{q}_{₁} is diagonal with real entries Raising both sides of (89) to a non-negative integer power k and simplifying thus results in

M^{1/2}(tA)^{k}M^{-1/2}=(M^{1/2}(tA)M^{-1/2})^{k}=S_{q}_{₁}(tL_{q}_{₁})^{k}S_{q}_{₁}⁻¹

which implies

$$\\ {M}^{{}^{} 1 / 2} {e}^{{}^{} tA} {M}^{{}^{} - 1 / 2} = {S}_{{q}_{1}} {e}^{{tL}_{{q}_{1}}} {S}_{{q}_{1}}^{{}^{} - 1} and hence$$

$$\\ {e}^{{}^{} tA} = {M}^{{}^{} - 1 / 2} {S}_{{q}_{1}} {e}^{{tL}_{{q}_{1}}} {S}_{{q}_{1}}^{{}^{} - 1} {M}^{{}^{} 1 / 2} .$$

Real Data Analysis (Section 5.2). This data set was collected by a SCRAM (Secure Continuous Remote Alcohol Monitor by Alcohol Monitoring Systems, Inc.) alcohol biosensor worn by a subject, which, by using fuel-cell technology, measures TAC in terms of local ethanol vapor concentration over the skin surface. Measurements were taken and recorded at non-equally spaced times. In addition, non equally spaced breath measurements were collected, at times that may not have coincided with those of the TAC.

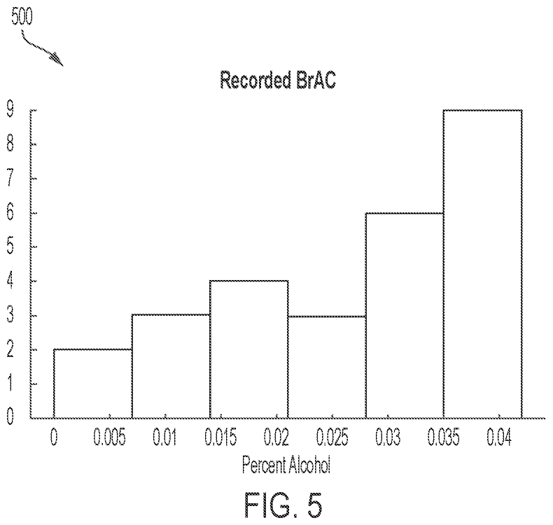

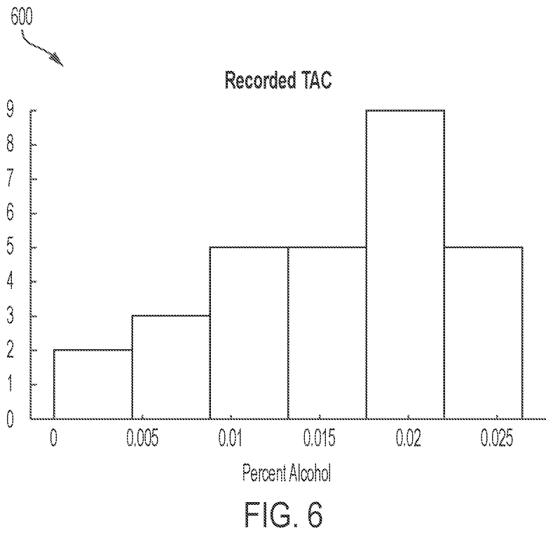

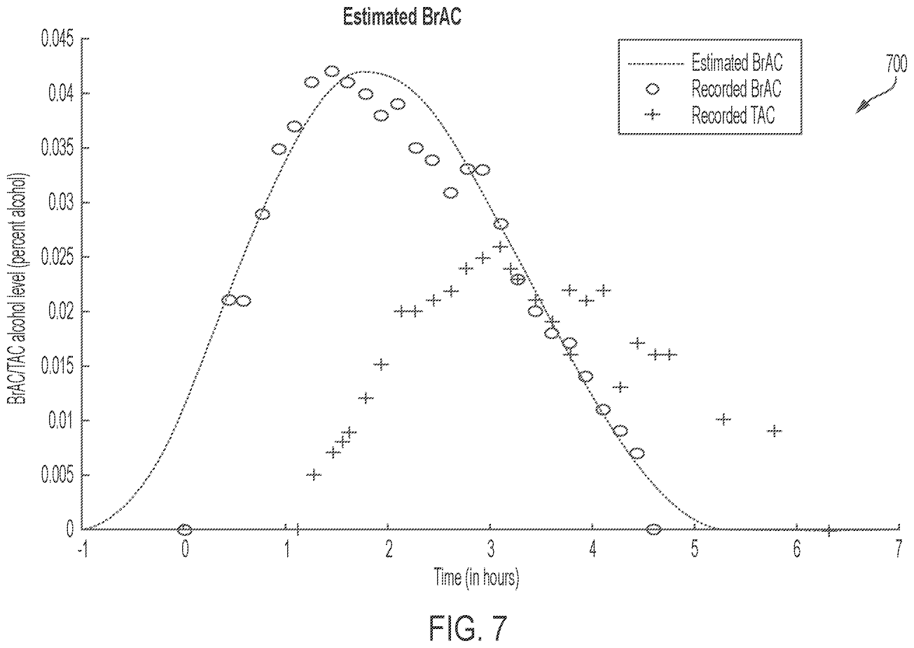

The data consists of 70 TAC and 28 BrAC observations collected during a single drinking session. The observations were taken over 6.3 hours and both TAC and BrAC observations were taken approximately every 10 minutes. BrAC was measured and recorded at the start of the drinking session and continued until it returned to 0.000. TAC was first measured 67 minutes after the first BrAC measurement and continued until it returned to 0.000. The TAC measurements provided by the sensor are in units of milligrams per deciliter (mg/dl), and the BrAC measurements are in units of percent alcohol. FIGS. 5 and 6 provide the range and distribution of the BrAC and TAC observations, which are labelled with this session's anonymized identifier BT311 Session1 06132019. FIG. 5 illustrates BrAC observations 500. FIG. 6 illustrates TAC observations 600. FIG. 7 illustrates a chart 700 of BrAC, TAC observations and estimated BrAC that results from using the minimizer \hat{q}=(0.6341,0.7826)

For the data analysis, we used k=4 in (26) and computed the matrices C, D, E and F as outlined there. We discretized the given time interval into 300 equal length sub-intervals. over each of which the BrAC is approximated as a constant value determined by interpolating to known BrAC values closest to the endpoints. Minimizing (6) resulted in the estimator \hat{q}=(0.5577,0.7550)

Further, we estimate the matrix Γ defined in (32) using \hat{q} in place of q in Lemma 3.3 to take the inner derivatives, and a Riemann sum approximation on the outer integral. Using the q and Γ estimates so obtained, and choosing an orthonormal basis in (47) to be such that the reconstructed BrAC curve returns the value 0 at the start of the drinking episode, we conclude via cross validation that a degree p=7 polynomial, computed using (47), provides the best fit to the BrAC curve. Lastly, βₙ(qₘ) and the estimated BrAC curve were calculated as in (52) and (54) respectively.

Uncertainty Quantification in Estimating Blood Alcohol Concentration from Transdermal Alcohol Concentration with Physics-Informed Neural Networks. Having discussed M-estimation in a diffusion model with applications for biosensor transdermal blood alcohol monitoring, the discussion will now shift to uncertainty quantification for the estimation of blood alcohol concentration using physics-informed neural networks. This model may be implemented in one or more embodiment of FIGS. 1A-B to determine BAC based on TAC. Specifically, we use a generative adversarial network with a residual-augmented loss function to estimate the distribution of unknown parameters in a diffusion equation model for a transdermal alcohol transport. We design another physics-informed neural network for the deconvolution of the blood alcohol signal from the transdermal alcohol signal. Based on the distribution of the unknown parameters, this network is able to estimate the blood alcohol signal and quantify the uncertainty in the form of credible bands. Finally, we show how a posterior latent variable can be used to sharpen these credible bands. We apply the techniques to an extensive data set of drinking episodes and demonstrate the advantages of this approach.

Producing meaningful quantitative measures of alcohol consumption in naturalistic settings is a challenging task for researchers and clinicians. Typically, they rely on the use of a breath alcohol analyzer or a drinker's self report. Unfortunately, both methods have their shortcomings: Obtaining deep lung samples (alveolar air) needed for accurate results can be difficult, and often alcohol contained in the mouth after drinking contaminates the results. Also, the procedure does not allow for continuous measurements. Self reports on the other band might be inaccurate as well, especially since it is known that alcohol directly affects the memory function of the brain.

Measuring the transdermal alcohol concentration (TAC) creates a possible alternative for the tracking of alcohol consumed. In recent years, biosensor devices for this purpose have been developed. The availability of TAC measuring devices allows for near-continuous measurements of alcohol consumption and helps researchers to gain insight into alcohol metabolism and drinking behavior. In addition. TAC sensors collect the data passively, i.e. contrary to self reports or breath alcohol analyzers no active participation of the subject is required

However, researchers interested in drinking behavior and alcohol consumption typically base their studies on breath alcohol concentration (BrAC) or blood alcohol concentration (BAC), and it was shown that BAC and BrAC reasonably agree. Hence, to make TAC sensors useful for alcohol research, the need to convert TAC signals to BAC/BrAC signals arises. Unfortunately, the direct conversion from TAC to BAC/BrAC proves to be difficult due to many confounding factors. Differences between devices and varying environmental conditions such as temperature and humidity lead to variations in the measurements. Intra- and inter-individual variations are another source that poses a challenge in the direct conversion from TAC to BAC/BrAC. The porosity and thickness of an individual's skin, the subject's drinking behavior, hydration and vasodilation are important factors in the functional relationship between BAC/BrAC and TAC.

There may be different approaches to overcome these difficulties. Some approaches use deterministic models for the relationship between BAC/BrAC and TAC. Some are based on regression models, while others model the transport of the alcohol from the blood through the epidermis by a one-dimensional diffusion equation with unknown parameters. Those parameters are then fit to an individual drinking session, known as an alcohol challenge. This method had two major caveats. First, this method required an alcohol challenge for each individual before the device is applied in the field and secondly, it did not account for the presence of natural variation and uncertainty. Indeed, parameters calibrated via an alcohol challenge could yield inaccurate results in a more naturalistic drinking setting. One data-driven, machine learning-based approach uses random forest-like, Extra-Trees.

Some approaches consider the unknown parameters as random variables and estimate their distribution by fitting a population model to a range of training data across varying subjects, devices and environmental conditions. Using the estimated distribution of the parameters it is not only possible to deconvolve the BAC/BrAC signal using the most likely parameter values, but conservative error bands can be obtained to measure and quantify the corresponding uncertainty. One work on this approach uses a least squares estimator based on naive pooled data, while another uses a Bayesian approach to find a posterior distribution of the parameters.

In various embodiments, work is disclosed herein below that relates to these approaches in that it yields a nonparametric distribution of the unknown parameters and conservative error bands for the deconvolved BAC/BrAC signal based on developments in the field of neural networks. Generative adversarial networks (GANs) are a class of neural networks that is able of generating artificial data with the same statistics as the training set. In this data-driven approach, large amounts of data are required to train the model. In clinical alcohol research, such data is typically not available. Indeed, as a result of the above mentioned difficulties, the acquisition of drinking session data is labor intensive and expensive. Moreover. by the very nature of the problem, only blood/breath alcohol and the transdermal alcohol can be measured. Data in the domain between blood vessels and skin is clearly unobservable.

To account for this situation, some treatments penalize the loss function of deep neural networks to incorporate physical knowledge of the problem into the training process. In some instances, a class of physics-informed neural networks (PINNs) was established. A framework for uncertainty propagation in physical systems may only allow for small training sets, but where prior information is available in the form of governing physical laws. In the present work, we aim to further develop this framework for the conversion of TAC to BrAC. Using a one-dimensional diffusion equation as a model for the alcohol transport through the epidermal layer of the skin, we train a physics-informed generative adversarial network with available drinking session data to yield estimates for the distribution of the unknown parameters. Then, in a second step, we employ a simple PINN for the deconvolution of the BAC/BrAC signal.

An outline of the remainder of the paper is as follows. We present our underlying mathematical model (Section 6) for alcohol transport through the epidermal layer of the skin. Then, we describe the probabilistic formulation and the generative adversarial network in detail (Section 7). We propose a physics-informed network for the deconvolution of the BAC/BrAC signal (Section 8). Then we demonstrate the efficacy and evaluate the performance of our approach through numerical studies using human subject drinking session data (Section 9).

Mathematical Model (Section 6) A family of first-principles, physics-based models have been proposed for the transport of ethanol through the epidermal layer of the skin. Common to all of these treatments is that fundamentally, they all rely on Fickian-diffusion as the underlying mechanism by which ethanol molecules propagate from the blood vessels in the dermal layer of the skin to the outer surface of the skin. Where the models do, on occasion, differ is in how they treat boundary phenomena. We note that modifying our general approach to accommodate any of the models would be straight forward.

In the following sections, the numbering of equations will begin again with (1). The corresponding system of equations, once the spatial and temporal variables have been transformed to be dimensionless, is given by

$$\frac{\partial x}{\partial t} ( t , η ) = {q}_{1} \frac{{\partial }^{2} x}{\partial {η}^{2}} ( t , η ) , 0 < η < 1 , t > 0 , \qquad ( 1 )$$

$${q}_{1} \frac{\partial x}{\partial η} ( t , 0 ) = x ( t , 0 ) , t > 0 , \qquad ( 2 )$$

$${q}_{1} \frac{\partial x}{\partial η} ( t , 1 ) = {q}_{2} u ( t ) , t > 0 , \qquad ( 3 )$$

$$x ( 0 , η ) = {x}_{0} , 0 < η < 1 , \qquad ( 4 )$$

$$y ( t ) = x ( t , 0 ) , t > 0. \qquad ( 5 )$$

Here, t is the temporal variable and η is the spatial variable, where η=0 is at the surface of the skin and η=1 is at the boundary between the epidermal and dermal layers of the skin. Note that dermal layer cells have an active blood supply, while epidermal cells do not. The alcohol concentration in the epidermis at time t and depth η is denoted by x(t, η), u(t) is the BrAC/BAC level and y(t) denotes the TAC level at the skin surface. Note that x(t, η) is inherently unobservable for η≠0. The parameter q₁ represents the normalized diffusivity of the epidermal layer and the parameter q₂ describes the flux gain from the blood alcohol. These parameters are unknown and, as described above, they vary between individuals and drinking episodes In the following, we assume (q₁, q₂) to be random and we aim to estimate their joint distribution.

Physics-Informed Adversarial Learning (Section 7). A probabilistic formulation is available for propagating uncertainty through physics-informed neural networks using latent variable models of the form x=x(t, η, z), z˜d, s.t. xₜ+ [symbol] _{η}x=0.

Here, z is the latent variable that has distribution d. We will assume d to be the standard normal distribution, but other continuous distributions are possible as well. Since z is a random variable, x(t, η, z) is a random field and we will write p(x|t, η, z) for the conditional density of x, knowing that t and η are deterministic. However, given data, t and η have some distribution in that data and so in this sense they can be assumed to be random and it is also possible to sample from those empirical distributions for t and η. Further, [symbol] _{η} is a general differential operator. The random field x is approximated as x(t, η, z)≈x_{θ}(t, η, z) by a deep neural network with the parameter set θ.

The main idea behind this approach is to combine all random effects and uncertainty into a single (possibly multidimensional) latent variable. That way, one can sample from the distribution of the latent variable z and propagate this through the neural network to yield samples of the random field x that reflect the uncertainty. To this end, the use of a generative adversarial net is proposed. Fundamentally, a GAN consists of two competing neural nets: The generator net tries to produce new data that is distributed as the training data. This new data is presented to the discriminator net that classifies the sample either as an actual sample or as a generated sample. Hence, the generator aims to fool the discriminator and the discriminator tries not to be fooled.

Kullerback-Leibler Based Training (Section 7.1). We use a learning mechanism for the generator that tries to match the joint distribution of the observed data q(t, η, x) with the joint distribution of the generated data p_{θ}(t, η, x) (the subscript θ denotes the parameters of the generator net). Such a matching can be achieved by minimizing the reverse Kullback-Leibler divergence of p_{θ}(t, η, x) and q(t, η,x). The Kullback-Leibler divergence is a measure of how different two distributions are, and by minimizing this divergence, we encourage the generator to produce samples that are distributed as the training data. The (reverse) Kullback-Leibler divergence is given by

$$𝕂𝕃 ( {p}_{θ} ( t , η , x ) ‖ q ( t , η , x ) ) := {𝔼}_{{p}_{θ} ( t , η , x )} ( log ( \frac{{p}_{θ} ( t , η , x )}{q ( t , η , x )} ) ) . \qquad ( 6 )$$

This can further be written as

[symbol]

(p_{θ}(t, η, x)∥q(t, ηx))=−H(p_{θ}(t, η, x))− [symbol] ₚ_{_{θ}}_{(t, η, x)}(−log(p(t, η, x))), (7)

where H(p_{θ}(t, η, x))= [symbol] ₚ_{_{θ}}_{(t, η,x)}(−log)p_{θ}(t, η, x))) denotes the entropy of the generator. In [44], the authors show that minimizing −λ·H(p_{θ}(t, η, x))− [symbol] ₚ_{_{θ}}_{(t, η, x)}(log(q(t, η, x))) with λ>1 instead of the pure Kullback-Leibler divergence introduces an entropic regularization to mitigate the common issue of mode collapse.

When minimizing (6) with respect to the generator parameters θ, we face the issue that we only have samples from the p_{θ} and the q distribution; the distributions themselves remain unknown. A general technique to approximate the density ratio of two distributions given only samples is based on a discriminator network T that acts as a binary classifier. Given N data points drawn from p_{θ}(t, η, x) labeled y=+1 and N data points drawn from q(t, η, x) labeled y=−1, the probabilities can be written as conditionals

p_{θ}(t,η,x)=α(t, η, x|y=1), q(t, η, x)=α(t, η, x|y=−1).

Then, the discriminator T is defined by T(t, η, x)=α(y=1|t,η,x) and using Bayes rule, the density ratio can be computed by

$$\begin{matrix}\frac{{p}_{θ} ( t , η , x )}{q ( t , η , x )} = \frac{α ( t , η , x \vert y = 1 )}{α ( t , η , x \vert y = - 1 )} \\ = \frac{α ( y = 1 \vert t , η , x )}{α ( y = - 1 \vert t , η , x )} \\ = \frac{T ( t , η , x )}{1 - T ( t , η , x )}\end{matrix} .$$

Another problem that arises when minimizing (7) is the computation of H(p_{θ}(t, η, x)) due to the fact that p_{θ}(t, η, x) is unknown a priori. Hence a computable lower bound for the entropy term is derived. Introducing a variational distribution q(z|t, η, x) represented by an encoder net q_{ϕ}(z|t, η, x) (the subscript ϕ denotes the parameters of encoder net), this bound reads

H(p_{θ}(t, η, x))≥H(z)+ [symbol] ₚ_{_{θ}}_{(t, η, x)}(log(q_{θ}(z|t, η, x))). (8)

Here, the variational distribution q(z|t, η, x) can be understood as a posterior distribution over the latent variable z, conditioned on t, η and x. We will return to this in section 3.4

Using this entropy bound and the method for estimating the density ratio based on samples, the following loss functions for minimization of the reverse Kullback-Leibler divergence can be defined:

[symbol]

_{D}(ψ)= [symbol] _{q(t,η)d(z)}(log(σ(T_{ψ}(t, η, x_{θ}(t, η, z))))) + [symbol] _{q(t,η,x)}(log(1−σ(T_{ψ}(t, η, x))) (9)

[symbol]

_{G}(θ, ϕ)= [symbol] _{q(t,η)d(z)}(T_{ψ}(t, η, x_{θ}(t, ηz))) +(1−λ)log(q_{ϕ}(z|t, η, x_{θ}(t, η, z))). (10)

Here, the subscript D denotes the discriminator loss, the subscript G denotes the generator loss, σ(y)=1/(1+e^{-y}) is the logistic sigmoidal function, and the subscript ψ denotes the parameters of the discriminator network. The subscripts in the expectation denote the corresponding distributions. That is, the subscript q(t, η)d(z) means that t and η are to be sampled from the empirical data distribution and z should be sampled from its prior d(z). It is clear that the generator aims to reduce the Kullback-Leibler divergence as much as possible, i.e. it strives for a minimum. The discriminator, on the other hand, tries to maximize its ability to correctly classify data samples and generated samples. This can be well seen in the discriminator loss. On the generated data samples, (t, η, x_{θ}(t, η, z)), the discriminator, T_{ψ}, should be large so that log(σ(T^{ψ})) becomes large, and on the empirical data samples. (t, η, x), the discriminator should be low so that log(1−σ(T^{ψ})) becomes large. Typically, such a model is trained by alternating between a minimization of the generator loss over the parameters θ and ϕ and a maximization of the discriminator loss over the parameters ψ.

Integration of the Physical Model (Section 7.2). Up to this point, the proposed method resembles a adversarial neural network. Typically, those networks are trained with large amounts of data. In our case, however, due to the expense of data collection and the unobservability of data in the regime η≠0, we only have a small training data set available. Thus, the pure data-driven approach of GANs will no longer work. We therefore resort to the idea of augmenting the above loss functions by information obtained from the physics of the problem. This is where the model from above proves to be of high value: The strong prior knowledge about the problem in form of a partial differential equation can be used to train the network. That way, a hybrid between pure data-driven approaches and physics-driven methods is created, a physics-informed neural network.

As a first step, we introduce two additional neural nets q₁_{_{μ}}(z) and q₁_{ᵥ}(z), i.e. we input the latent variable into these nets to propagate the uncertainty though the estimates of q₁ and q₂. Now, the physics of the problem can be integrated in the training process by introducing a PDE-related loss function. To this end, we specify Nᵣ_{ᵢ} collocation points in the interior of the domain {(t, η):t>0, 0<η<1}, Nᵣ_{_{b1}} collocation points on the left boundary {(t, η):t>0, η=0} and Nᵣ_{_{b2}} collocation points on the right boundary {(t, η):t>0, η=1}. We then compute the residual of the PDE at these collocation points (tⱼ, ηⱼ) in dependence of the parameters θ of the generative model and the parameters μ and v of the parameter-estimating networks as

$${ℒ}_{PDE} ( θ , μ , 𝓋 ) = \frac{1}{{N}_{{r}_{i}}} {\sum }_{j = 1}^{{N}_{{r}_{i}}} {( \frac{\partial {x}_{θ}}{\partial t} ( {t}_{j} , {η}_{j} ) - {q}_{{1}_{μ}} \frac{{\partial }^{2} {x}_{θ}}{\partial η} ( {t}_{j} , {η}_{j} ) )}^{2} + \frac{1}{{N}_{{r}_{b 1}}} {\sum }_{j = 1}^{{N}_{{r}_{b 1}}} {( {q}_{{1}_{μ}} \frac{\partial {x}_{θ}}{\partial η} ( {t}_{j} , 0 ) - {x}_{θ} ( {t}_{j} , 0 ) )}^{2} + \frac{1}{{N}_{{r}_{b 2}}} {\sum }_{j = 1}^{{N}_{{r}_{b 2}}} {( {q}_{{1}_{μ}} \frac{\partial {x}_{θ}}{\partial η} ( {t}_{j} , 1 ) - {q}_{{2}_{𝓋}} u ( {t}_{j} ) )}^{2} . \qquad ( 11 )$$

Note that in this formulation, we treat the residual as a deterministic value, i.e. we set x_{θ}(t, η, z)=x_{θ}(t, η) as well as q₁_{_{μ}}(z)=q₁_{_{μ}} and q₁_{ᵥ}(z)=q₁_{ᵥ}. The gradients appearing in these residuals can be efficiently evaluated thanks to the recent advances in automatic differentiation. Therefore, no discretization schemes for the differential operators are required. Also note that the initial condition (4) could be included in this PDE loss. However, we choose to account for that using the Kullback-Leibler divergence based training process of the generator.

Now, we augment the generator loss with a scaled version of the PDE loss as

[symbol]

_{G}(θ, ϕ)+β [symbol] _{PDE}(θ, μ, v). (12)

That way, for β>0, the introduced PDE residual acts as a regularization term that leads the generator to create samples that approximately satisfy the diffusion equation model (1)-(5). The precise choice of β is a tuning between the dominance of the data on the one hand, and the dominance of the physics on the other. As shown herein, the value of β influences the result, so it has to be chosen experimentally to yield a good balance between data and physics.

We also want to emphasize that the augmentation of the generator loss with the physics information is the core element in the estimation of q₁ and q₂. By minimizing the combined loss, the network parameters μ and v are also adjusted such that the obtained estimates q₁_{_{μ}}(z) and q₂_{ᵥ}(z) match the training data as well as the first principles physics based model (1)-(5) in an optimal fashion.

Combining all of this together and using the loss functions, we ultimately obtain the following minimax problem for the generator and the discriminator

$${max}_{ψ} {ℒ}_{D} ( ψ ) {min}_{θ , ϕ , μ , 𝓋} {ℒ}_{G} ( θ , ϕ ) + {βℒ}_{PDE} ( θ , μ , 𝓋 ) . \qquad ( 13 )$$

To see how the observable data for TAC and BAC/BrAC enter the training process, note that in the diffusion model (1)-(5), the TAC data acts as a Dirichlet output. We can thus directly use the TAC data as training data for the generator. The handling of the BAC/BrAC data, however, is more involved. The BAC/BrAC is a Neumann-type input of (1)-(5) and so it is not represented by x_{θ}(t, η, z) for some values of t and η. Hence, we only incorporate the BAC/BrAC data using the PDE loss. So the model is encouraged to match the distribution of the TAC data by minimizing the Kullback-Leibler divergence and it is encouraged to match the BAC/BrAC data and to obey the physical model by minimizing the residuals of the equations. The interplay between those two objectives is governed by the tuning parameter β.

Estimating the Parameter Distribution (Section 7.3). After training the generative model. it remains to estimate the resulting distribution of (q₁, q₂) By design, q₁_{_{μ}} and q₂_{ᵥ} depend on the latent variable z. Thus, we can sample from the latent variable and pass these samples through the networks for q₁ and q₂ in order to obtain samples of these parameters. Using a sufficiently large number of samples, we can create an estimate of the distribution of (q₁, q₂). Hence, the presented method not only estimates the distribution of (q₁, q₂), but the trained networks can directly be used to generate samples of this distribution by a simple forward-pass. That way, we can avoid the use of sampling algorithms like Markov chain Monte Carlo.

Posterior Distribution of the Latent Variable (Section 7.4). As mentioned, the entropic regularization requires the introduction of an additional encoder network q_{ϕ}(z|t, η, x). While this might seem like a complication to the model which in and of itself is not all that useful, in fact, the encoder offers a remarkable advantage. During the training process, the encoder network learns the best, i.e. most likely, latent variables given the data. So, based on the TAC and BAC/BrAC data, the encoder network yields a posterior distribution over the latent variable conditioned on the training data. Moreover, since the encoder network is involved in the training of the generator which is physics-informed, the posterior for the latent variable will also be physics-informed. Thus, as a byproduct, we obtain a posterior distribution of the latent variable that is both data- and physics-informed. In the context of our given problem, this is very appealing. Instead of a direct sampling from the prior for the latent variable and the subsequent passing the samples through the parameter networks q₂_{_{μ}} and q₂_{ᵥ} , this allows us to pass all available data through the encoder, q_{ϕ}, to obtain a posterior distribution of the latent variable. This distribution can then be fed to the parameter networks to yield an estimated distribution of (q₁, q₂). We examine the use of this posterior latent distribution herein.

Network Design (Section 7.5). The accuracy of the model is highly dependent on the architecture of the network. The formulation of the given problem only allows for observable data at η=0. i.e. the TAC data, and additionally the BAC/BrAC data. Points inside the domain are inherently unobservable, hence they cannot be used as training data Consequently, the training data is very sparse. A formulation allowing for only a relatively few training data favors the discriminator network, i.e. it is easy to classify samples into generated samples and actual samples. However, when the discriminator network is too strong, the generator gains little information from the discriminator and the ability of the generator network to learn is impaired.

To account for this, we use a discriminator network with a low capacity compared to the other networks. This can be achieved in two different ways: First, we can decrease the capacity of the discriminator by choosing a network design that involves fewer hidden layers and neurons per layer. Secondly, we can strengthen the generator by allowing more learning steps in the alternating learning process. That way, we enable the generator to train a certain number of steps for a given discriminator before the discriminator improves further.

Deconvolution of the Input Signal (Section 8). After estimating the distribution of (q₁, q₂), the next step is to deconvolve the BAC/BrAC signal from the TAC signal. Here, we want to employ a simple physics-informed neural network for the deconvolution process. Given the TAC signal, the output of the network is x_{φ}(t, η, q₁, q₂), the (unobservable) alcohol level in the epidermal layer. To this end, we use the only available training data, the TAC signal consisting of N_{T} data points, and set up the TAC-related loss

$${ℒ}_{𝒯} ( φ ) = \frac{1}{{N}_{r}} {\sum }_{i = 1}^{{N}_{T}} {( {x}_{φ} ( {t}_{i} , 0 ) - y ( {t}_{i} ) )}^{2} .$$

This way, the network is encouraged to match the provided TAC signal at η=0.

Using the penalty approach for the incorporation of the PDE described above, we augment the loss by a PDE-related loss

$${ℒ}_{PDE} ( φ ) = \frac{1}{{N}_{{r}_{i}}} {\sum }_{j = 1}^{{N}_{{r}_{i}}} {( \frac{\partial {x}_{φ}}{\partial t} ( {t}_{j} , {η}_{j} ) - {q}_{1} \frac{{\partial }^{2} {x}_{φ}}{\partial η} ( {t}_{j} , {η}_{j} ) )}^{2} + \frac{1}{{N}_{{r}_{b 1}}} {\sum }_{j = 1}^{{N}_{{r}_{b 1}}} {( {q}_{1} \frac{\partial {x}_{φ}}{\partial η} ( {t}_{j} , 0 ) - {x}_{φ} ( {t}_{j} , 0 ) )}^{2} . \qquad ( 14 )$$

The complete loss is now given as [symbol] = [symbol] ᵣ+β· [symbol] _{PDE}. Once the network is trained for the specific drinking episode using the TAC signal, the BAC/BrAC signal can then be estimated using equation (3) and automatic differentiation. Note that q₁ and q₂ are inputs of the network and are included in the training process. So, in order to obtain BAC/BrAC estimates for varying values of q₁ and q₂, only a simple forward pass through the network is required. This enables us to directly use the available sample of the joint distribution for (q₁, q₂) to produce BAC/BrAC estimates based on this sample. Hence, it is easy and time-efficient to come up with conservative error bands. In our discussions below, in the interest of brevity, we will refer to these conservative error regions simply as error regions.

Numerical Results (Section 9). The computations we report on here were based on a set of 150 recorded drinking episodes gathered in the laboratory. In each drinking episode, the BAC/BrAC signal was recorded as well as a TAC signal from two different biosensors. Some of those drinking sessions were recorded using a different test protocol, i.e. the TAC sensor was worn on a leg instead of an arm. We removed those sessions, so that we are left with a set of 126 drinking episodes as the basis for our numerical studies. All algorithms were implemented in Tensorflow and the corresponding computations were executed on a NVIDIA Tesla T4 GPU card.

The GAN model was trained for 50,000 iterations using the Adam optimizer. The learning rate was set to 10⁻⁴ and the ratio for the generator and discriminator updates was set to 10. For the entropic regularization we used the value λ=1.5 which was found to be suitable in prior works. If not stated explicitly, the penalty parameter β=1 was used. In some examples however, we used β=4 to reflect the fact that the problem is more physics-driven than data-driven. The dimension of the latent variable space was chosen to be 1. The network topology for the generator and the encoder consisted of four hidden layers with 50 neurons each, whereas the discriminator network only had one hidden layer with 20 neurons. As was indicated, this accounts for the small number of available training data sets. The networks for q₁ and q₂ each have two hidden layers with 50 neurons. We used N_{b}=126,252 boundary training data together with Nᵢ=20,000 initial training data, i.e. Nᵤ=146,252, and Nᵣ_{ᵢ}=Nᵣ_{_{b1}}=Nᵣ_{_{b2}}=50,000 collocation points. In every iteration, a batch of 5,000 training data points and 500 collocation points was randomly chosen to compute the loss functions. Once the model was trained, we sampled 100,000 values of the joint distribution of (q₁, q₂)using a standard normal distribution for the prior of the latent variable. We also fed the Nᵤ=146,252 data points to the encoder network to get samples of the posterior distribution of the latent variable. These samples were consequently fed into the networks for q₁ and q₂ to produce a posterior joint distribution of (q₁, q₂).

The deconvolution network was trained for 30,000 iterations using the Adam optimizer. This network had five bidden layers with 50 neurons each. We used Nᵣ_{ᵢ}=Nᵣ_{_{b1}}=50,000 collocation points of which 500 were chosen randomly in every iteration. The penalty parameter was set to β=10.

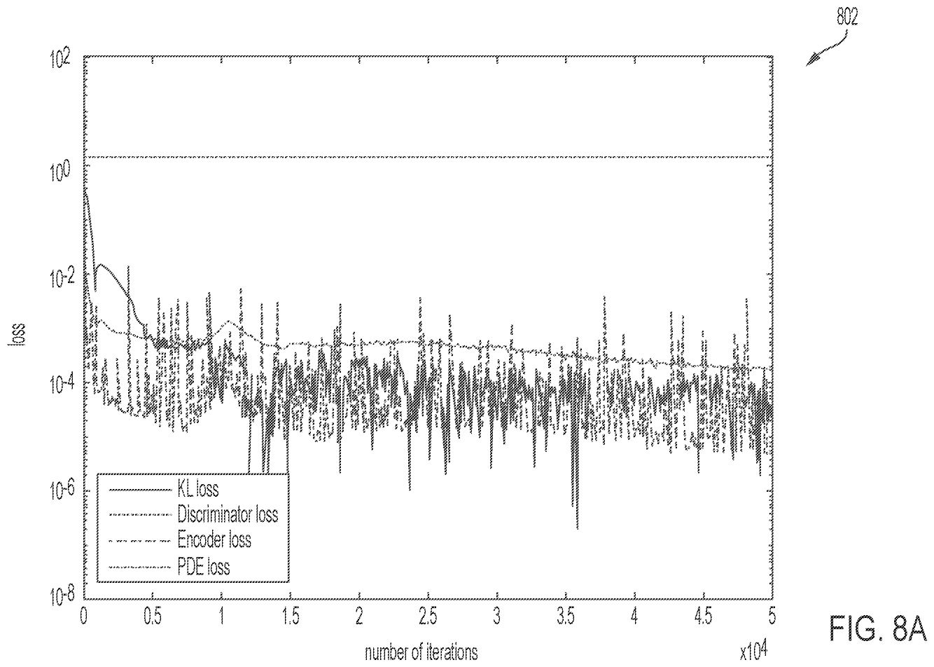

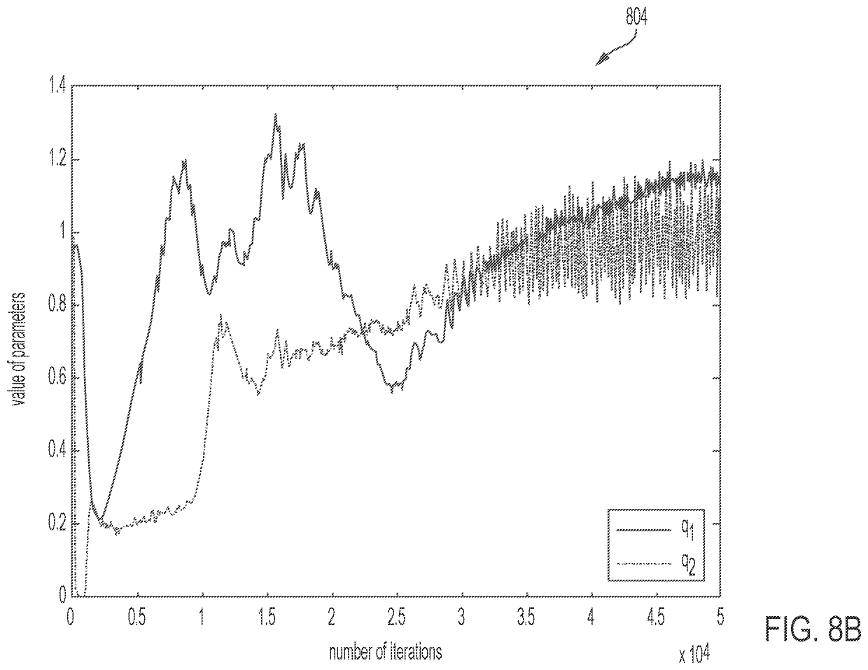

FIG. 8A shows the values 802 for the loss functions over the number of iterations. This figure illustrates values of different parts of the loss function during the training of the GAN model. The curves for the Kullback-Leibler divergence and the encoder loss show many outliers, whereas the PDE loss decreases quite steadily. However, due to stochastic gradient descent using batches of data in every iteration, the convergence is not monotonic. The discriminator loss quickly reaches a maximum value and remains constant over the iterations. This makes sense as the discriminator loss is to be maximized.

FIG. 8B shows the values 804 of q₁ and q₂ over the number of iterations. Here, we pass a 5,000-sample of a standard normal through the networks for q₁ and q₂ and display the mean value. Comparing this, we see that the parameter values start to converge relatively late. The fluctuating shape of the curves in the converged state is due to the probabilistic nature of taking samples.

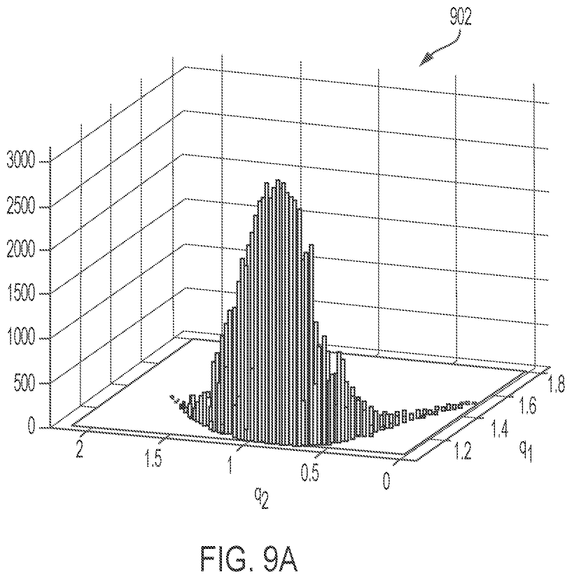

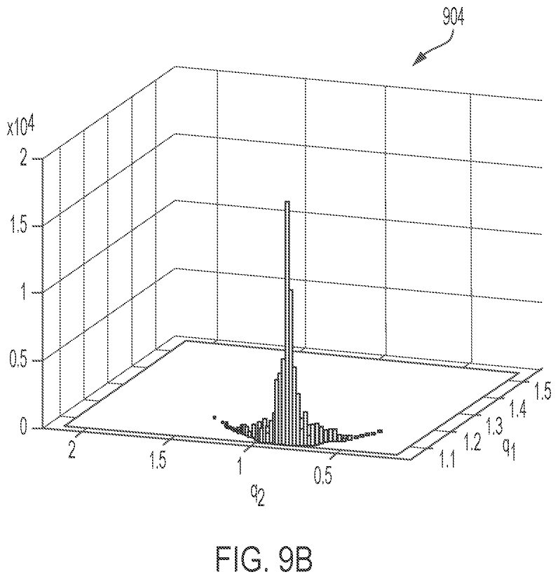

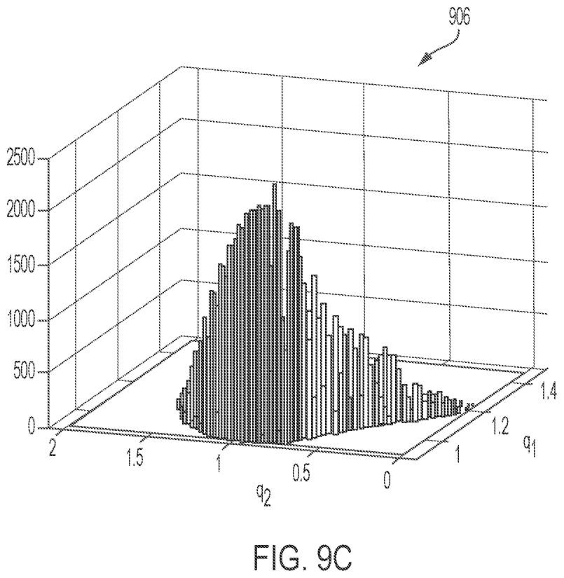

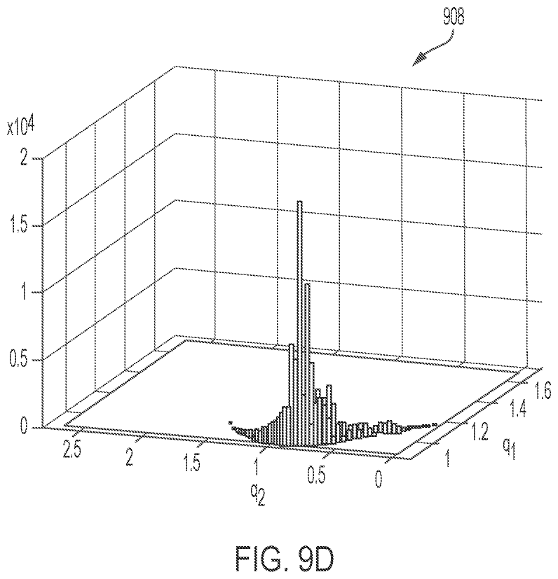

One of the main goals of this work is to estimate the distribution of the random parameters (q₁, q₂). FIGS. 9A-D show histograms of the joint distribution. We depict the distribution using the full data of 126 drinking sessions. FIG. 9A shows that distribution 902 for 88 selected drinking episodes using a standard normal distribution for the prior of the latent variable FIG. 9B shows a distribution 904 using the posterior over the latent variable. FIG. 9C displays the distribution 906 for the full set of 126 drinking episodes with a standard normal prior for the latent variable. FIG. 9D shows the corresponding distribution 908 using the posterior distribution for the latent variable. In various cases, the histogram appears to be a curve in the two-dimensional parameter space. This also proved to be true using a two-dimensional latent variable. It is apparent that the histogram using 88 drinking episodes is narrower than the histogram using all available data. It appears that the greater variability of the full data is directly reflected in the estimated parameter distribution.

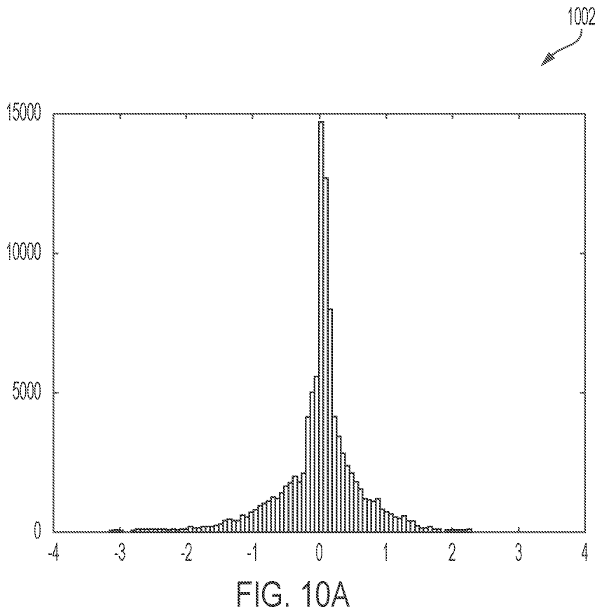

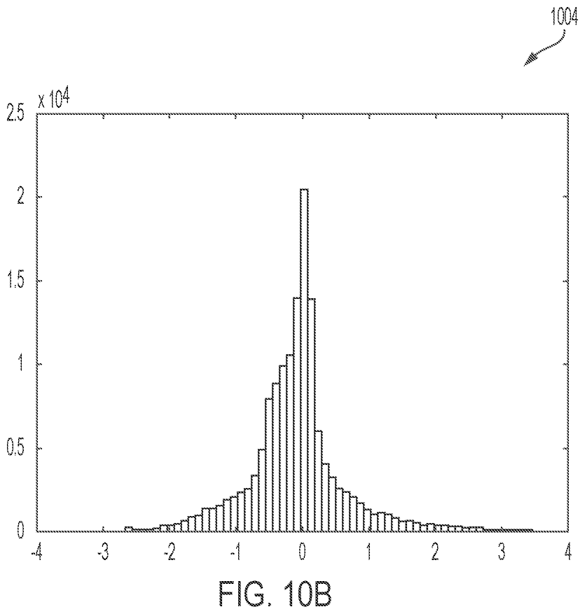

The histogram of the posterior latent variable is given. FIG. 10A shows a distribution 1002 of the posterior latent variable with a historggram for 88 drinking sessions used as training data and FIG. 10B shows the histogram 1004 for 126 drinking sessions used as training data. We see that this distribution decays much more rapidly than a standard normal. Hence, using this distribution as input for q₁ and q₂ we expect a more concentrated distribution for those parameters. For the histograms with the joint distribution using samples of the posterior latent distribution as input, the histograms are much more centered around the most likely parameter values. This supports the idea that the posterior latent variable can indeed be used to yield sharper error bands for the BAC/BrAC signal.

TABLE XX
Estimated Parameter Statistics

| | latent | | Mean | Mean | Radius of |
|---|---|---|---|---|---|
| m | variable | β | value q₁ | value q₂ | error circle |
| 88 | prior | 1 | 1.0935 | 0.8528 | 0.5148 |
| 88 | posterior | 1 | 1.0639 | 0.8527 | 0.3256 |
| 126 | prior | 1 | 0.9573 | 0.8083 | 0.5766 |
| 126 | posterior | 1 | 0.9430 | 0.7915 | 0.4678 |
| 126 | prior | 4 | 1.1764 | 1.1582 | 0.6620 |

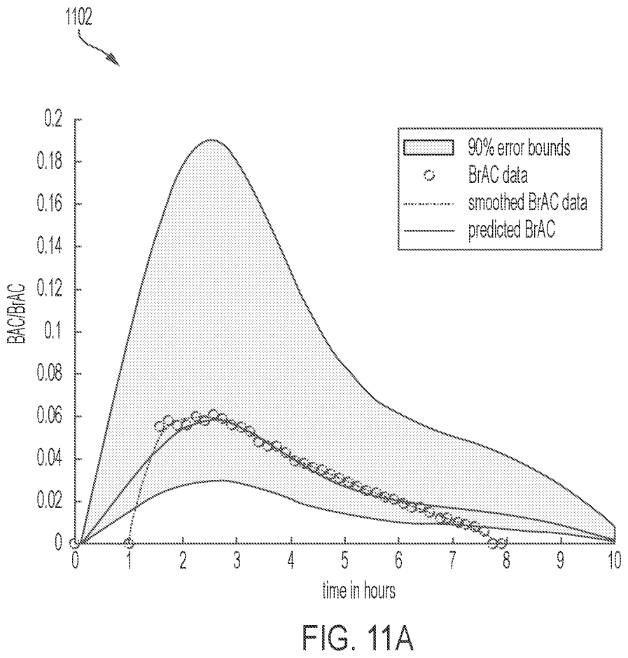

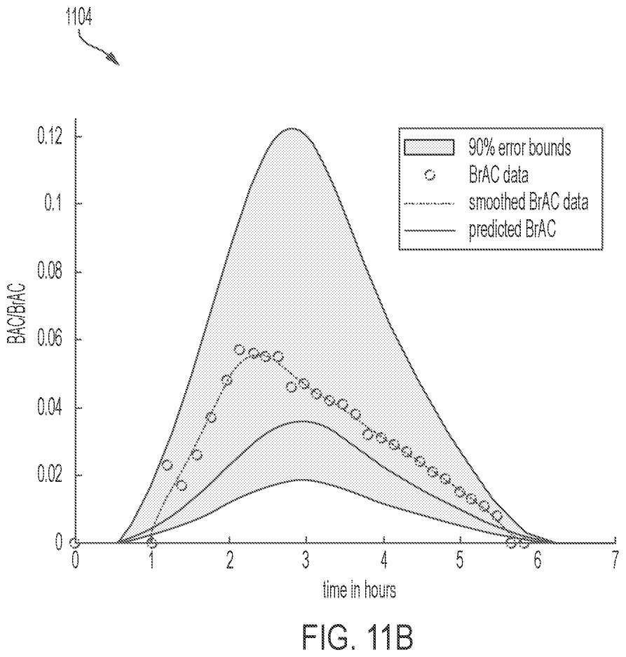

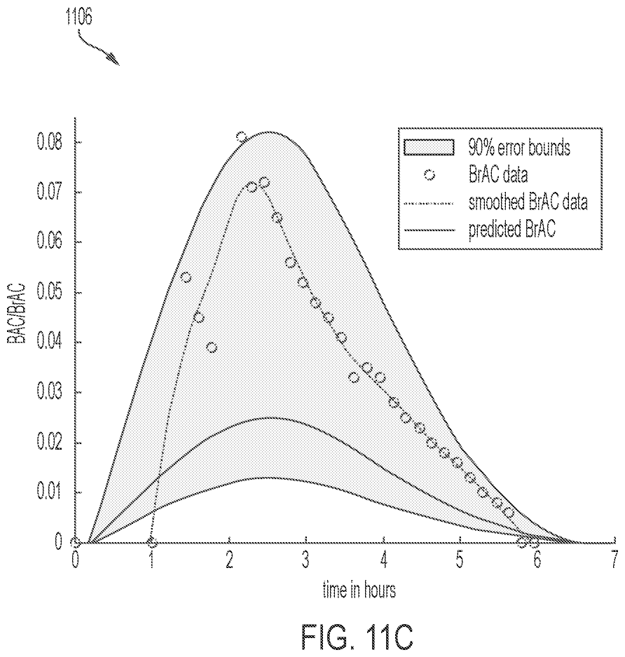

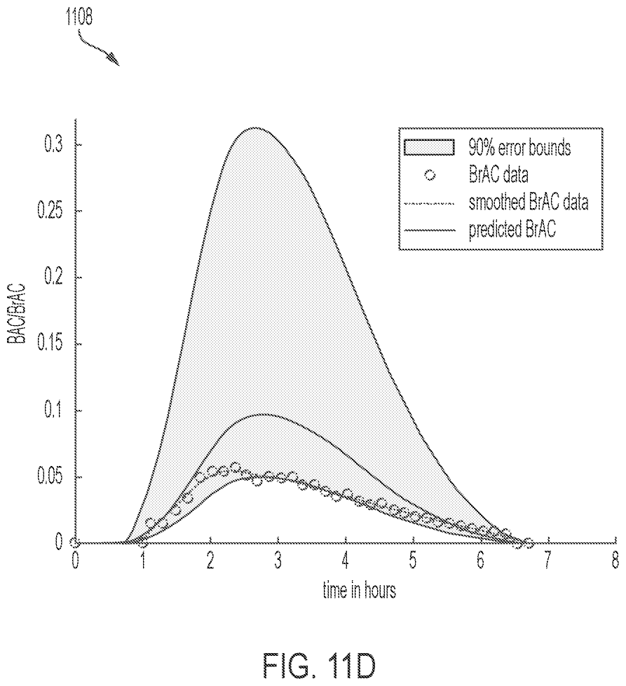

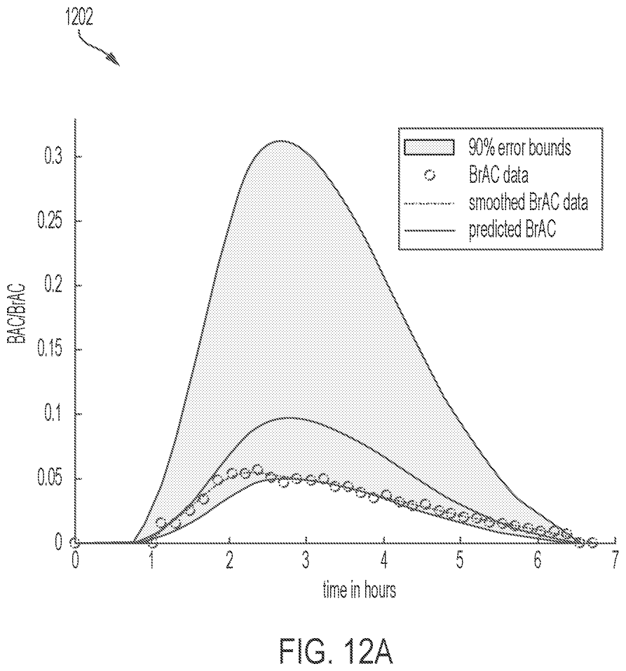

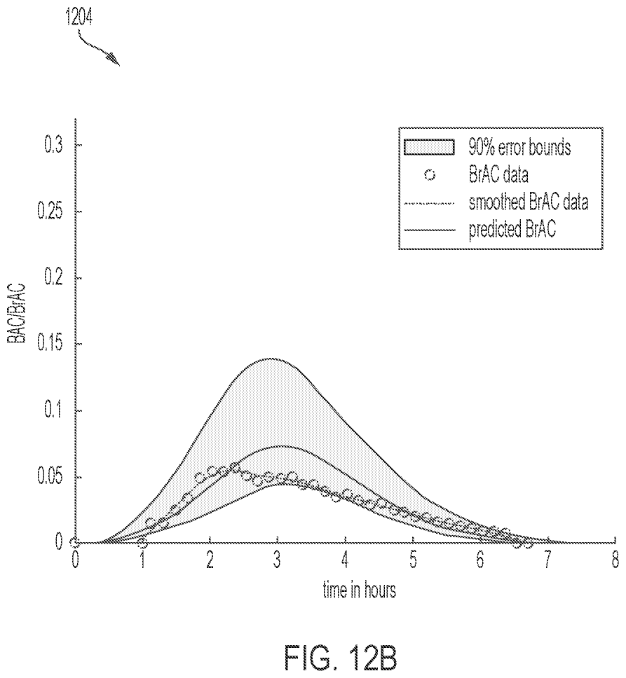

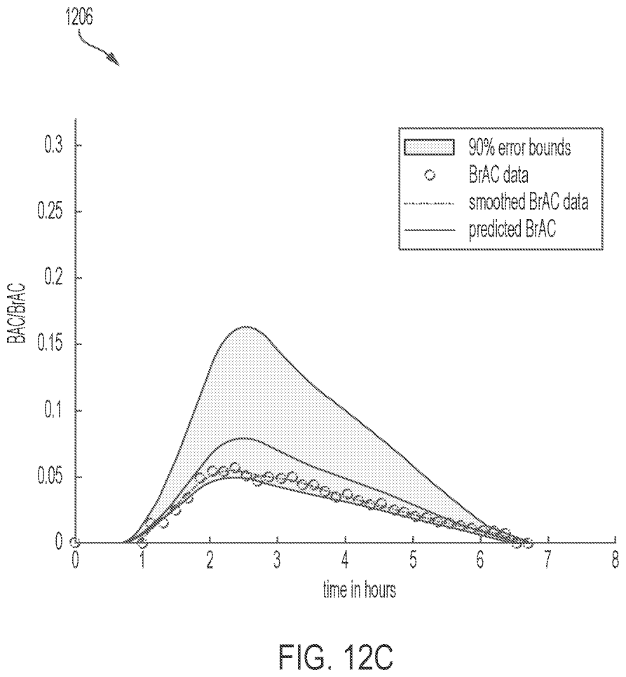

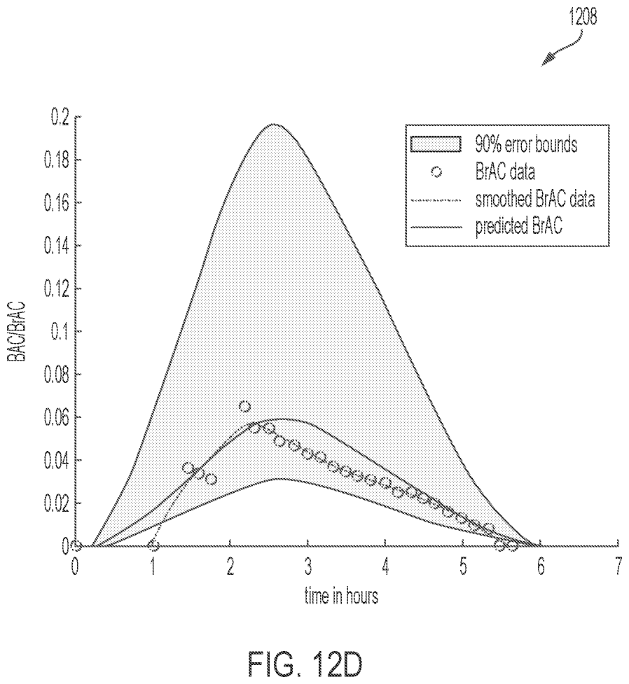

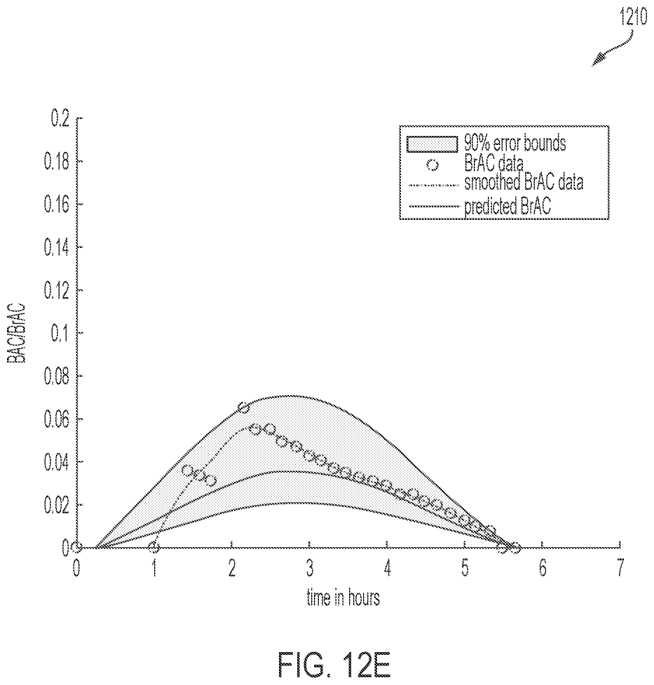

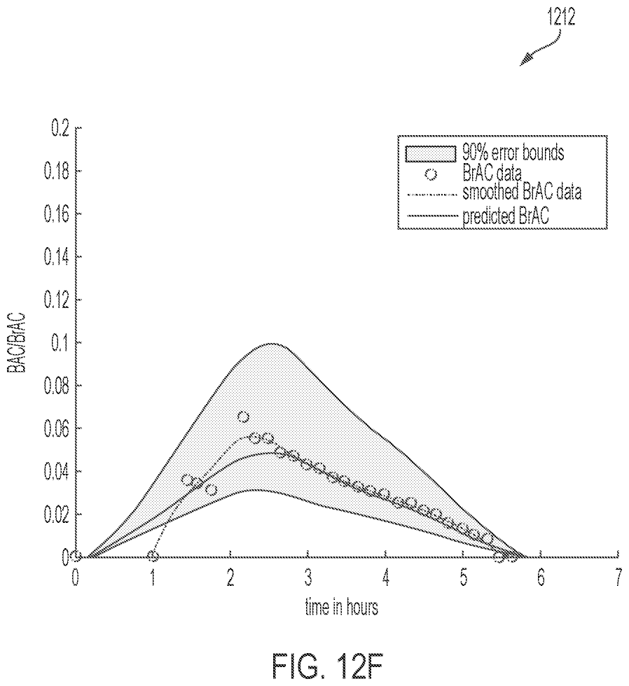

Using the estimated joint distribution for (q₁, q₂), we can use the deconvolution network described above to recover the BAC/BrAC signal and to find error bands. By sampling the joint distribution, we compute the mean parameter values to get the mean predicted BAC/BrAC signal. We also take a radius around this mean such that 90 per cent of the samples fall into this circle. Then, we use these samples to find the BAC/BrAC predictions corresponding to the parameter values. Note that after training the deconvolution network, this process only requires forward passes through the network. At each time, we use the maximal and the minimal value of these predicted signals to form conservative error bands. FIG. 11A-D show these results for four selected drinking episodes using the parameter distribution from training the GAN with all 126 drinking episodes. FIG. 11A and 11B show two examples (1102, FIG. 11A and 1104, FIG. 11B) of a situation where the mean prediction matches the real signal quite well. We notice, however, that the method yields a predicted start of the BAC/BrAC curve that is smoother than the real data. The sudden jump in the signal at the beginning of a drinking episode is not well reflected. It is also visible that the error region appears to be rather large in both cases. This is due to the fact that the data exhibits high variability across subjects and drinking episodes. Indeed, the larger error region is required to capture this variability as shown in graph 1104 (FIG. 11C) and graph 1106 (FIG. 11D). Even though the nature of the data is to vary across subjects and drinking episodes, it is desirable to keep the error bands small. One way to achieve this is to use the posterior distribution of the latent variable in the generation of samples for (q₁, q₂) rather than the prior standard normal. As seen in FIG. 12F, the joint distribution 1212 of the parameters becomes narrower in this case. Another way appears to be the tuning of the penalty parameter β. The default choice of β=1 leads to a balance between given training data and physics. As described, the underlying problem is rather driven by physics and so a higher weight on the PDE residuals might be favorable. FIGS. 12A-F compare these different approaches 1202, 1204, 1206, 1208, 1210, and 1212 for two different drinking sessions. It shows that both ways lead to narrower error bands. Note that this does not necessarily improve the quality of the mean prediction: In FIG. 12D, the approach 1208 and specifically the default mean prediction matches the actual data nicely, whereas the approach 1210 and match in FIG. 12E using the posterior latent is worse although the error region is smaller.

FIG. 12A-F shows a comparison of predicted BAC/BrAC signals using two different drinking episodes. FIGS. 12A and 12D show the estimated BAC/BrAC curves 1202, 1208 yielded by a standard normal distribution for the prior of the latent variable and β=1. FIGS. 12B and 12E show the corresponding results 1204, 1210 using the posterior distribution of the latent variable and β=1. FIGS. 12C and 12F display the corresponding results 1206, 1212 using a standard normal distribution for the prior of the latent variable together with β=4.

In this work, we have proposed a stochastic physics-informed generative adversarial network for the estimation of an unknown parameter distribution in the context of an input/output model for the transport of alcohol through the epidermal layers of the skin. Based on these estimated distributions, we designed a simple physics-informed network for the deconvolution of the BAC/BrAC signal from the TAC signal. Our approach using physics-informed learning techniques is novel in the realm of this application. The stochasticity of this approach further allowed us to obtain error bands for the estimated signal. Moreover, we employed an encoder network, introduced as an entropy regularization, to gain a posterior distribution over the latent variable which provides a means to sharpen the error region. Finally, we demonstrated the performance of this method with a range of numerical examples using a human subject data set consisting of 126 drinking episodes.

Discrete-Time Linear Quadratic Gaussian Control and Estimation Compensator. Continuing the discussion of determining blood alcohol concentration based on TAC, attention now moves to a discrete-time linear quadratic gaussian (LQG) control and estimation compensator for random abstract parabolic systems. This compensator may be implemented in one or more embodiment of FIGS. 1A-B to determine BAC based on TAC.

A finite-dimensional approximation and convergence theory for the closed-loop linear quadratic control and estimation of abstract parabolic systems with random parameters is developed. The motivation for this effort is the development of a real-time control scheme for intravenous infused alcohol studies based on a population model for the transdermal transport of alcohol and a transdermal alcohol biosensor that measures the ethanol content in perspiration. We apply Galerkin-based approximation to a weak formulation of the underlying random parabolic system in appropriately constructed Bochner spaces wherein the random parameters are treated as additional spatial variables. Our LQG optimization, approximation, and convergence results are argued using results from linear semigroup theory. An example and results from some of our numerical studies are included.

We develop a finite-dimensional approximation and convergence theory for the discrete-time linear quadratic Gaussian (LQG) control and estimation of abstract parabolic systems with random parameters. There are two primary motivations for this study. The first is the development of real-time closed-loop feedback for human subject laboratory studies involving the intravenous infusion of alcohol based on transdermal sensing, and the second is the development of an efficient, real-time, deconvolution scheme for a population model for the transdermal transport and measurement of alcohol. In both instances the underlying dynamical model takes the form of an abstract semi-linear, parabolic partial/ordinary differential equation (PDE/ODE) hybrid system describing the transport of ethanol from the blood through the skin, its excretion via perspiration, and finally its measurement on the surface of the skin by an electro-chemical biosensor (in actuality, a fuel cell) worn on the ankle or the wrist. In the first application, the control input to the model is the intravenously infused alcohol and in the second it is either blood or breath alcohol concentration (BAC/BrAC). The output is transdermal alcohol concentration (TAC). The goal in the control problem is to “clamp” the blood alcohol concentration at a predetermined (typically) constant level, while the goal of the deconvolution problem is to estimate BAC/BrAC from the biosensor measured TAC. Although the model captures the underlying physics quite well, the parameters can vary with the individual wearing the sensor, the particular sensor being worn, and environmental factors such as ambient temperature and humidity. This variation is dealt with by allowing the model parameters to be random with either known or estimated distribution, the result being a population model. In this paper we focus on the control problem and formulate it as an LQ regulator coupled with an LQG estimator or observer which together are known as an LQG compensator. We formulate the deconvolution problem as an LQG tracking problem and will report on our results for it in a subsequent paper.

The approximation theory for the continuous-time LQR problem in Hilbert space was developed and specifically for abstract parabolic systems For discrete-time LQR problems in Hilbert space, LQR approximation results can be found. The finite-dimensional approximation and convergence theory for the discrete-time LQG compensator in Hilbert space was developed. Here we investigate the application of these results into abstract parabolic systems with random parameters by exploiting some more recent results on systems of this type. In these treatments, the underlying parabolic systems are considered in weak form in appropriately constructed Bochner spaces wherein the random parameters are effectively treated as additional spatial variables. In this way their LQ control and estimation can be formulated in Hilbert space and their finite-dimensional approximation can be facilitated via a Galerkin approach. The closed-loop linear state feedback solution to the resulting LQG compensator problem and convergence results for the finite-dimensional approximations can be argued with the aid of linear semigroup theory.

An outline of the remainder of the discussion is as follows. In Sections 10, 11 and 12 we briefly outline the optimization, approximation, and convergence theory for the discrete-time LQR and LQG compensator problems in Hilbert space. In Section 13 we discuss the weak formulation of abstract parabolic systems with random parameters. In Section 14 we show how the LQR results in Sections 10 and 11 can be applied to systems of the form discussed in Section 13. In Section 15 we treat the control problem for the intravenous infusion of ethanol involving the transdermal alcohol biosensor and present the results of some of our numerical studies followed by some discussion and a few concluding remarks.

The Discrete-Time Linear Quadratic (Section 10). Let X, Y and U be separable Hilbert spaces with inner products [symbol] ⋅, ⋅ [symbol] _{X}, [symbol] ⋅, ⋅ [symbol] _{Y} and [symbol] ⋅, ⋅ [symbol] _{U}, respectively. Let Â∈ [symbol] (X, X), \hat{B}∈ [symbol] (U, X) and Ĉ∈ [symbol] (X, Y). Let \hat{Q}∈ [symbol] (X, X) and Ĝ∈ [symbol] (X, X) be positive semi-definite self-adjoint and let \hat{R}∈ [symbol] (U, U) be positive definite self-adjoint. Let \hat{B}₁∈ [symbol] ( [symbol] ^{μ}, X), Ĉ₁∈ [symbol] ( [symbol] ^{v}, Y) and consider a discrete-time linear dynamical system given by:

xₖ₊₁=Âxₖ+\hat{B}uₖ+\hat{B}₁ωₖ, k≥k₀, xₖ_{₀}=x₀,

yₖ=Ĉxₖ+Ĉ₁ζₖ

together with a quadratic performance index on the finite-time horizon [k₀, k₁]:

$$\hat{J} ( u ) = {\sum }_{k = {k}_{0}}^{{k}_{1} - 1} {〈 \hat{Q} {x}_{k} , {x}_{k} 〉}_{X} + {〈 \hat{R} {u}_{k} , {u}_{k} 〉}_{U} + {〈 \hat{G} {x}_{{k}_{1}} , {x}_{{k}_{1}} 〉}_{X}$$

In the system above {ωₖ} and {ζₖ} denote respectively [symbol] ^{μ} and [symbol] ^{v} valued uncorrelated, zero-mean, stationary, Gaussian white noise processes with each component having common variance σ_{Q}² (state) and σ_{K}² (output) that corrupt the state and measurement through the operators \hat{B}₁ and Ĉ₁. We interpret the Hilbert space valued stochastic perturbations to the state and output equations in the usual sense with respect to an orthonormal basis yielding the state and output covariance operators σ_{Q}²\hat{B}₁\hat{B}*₁ and of σ_{K}²Ĉ₁Ĉ*₁, respectively.

The deterministic time-invariant finite-horizon discrete-time linear quadratic regulator control problem is given by:
(P1) Choose an input ū∈l²(k₀, k₁−1; U) for which the criterion (2) is minimized.

A control sequence u∈l²(k₀, ∞; U) is defined to be an admissible control for the initial condition x₀ if Ĵ(u)<∞. Then consider a quadratic performance index given by:

$$\hat{J} ( u ) = {lim}_{{k}_{1} - ∞} {\sum }_{k = {k}_{0}}^{{k}_{1}} {〈 \hat{Q} {x}_{k} , {x}_{k} 〉}_{X} + {〈 \hat{R} {u}_{k} , {u}_{k} 〉}_{U} .$$

The deterministic time-invariant infinite-borizon discrete-time linear quadratic regulator control problem is given by:
(P2) Choose an input ū∈l²(k₀, ∞; U) for which the criterion 3 is minimized, if an admissible control exists for the initial condition x₀

The closed-loop solutions to these discrete-time LQR control problems in linear state feedback form are given. For every initial value x₀, the optimal input for the problem (P1) is unique and generated by the linear control law ūₖ=−Fₖx̅ₖ, k=k₀, k₀+1, . . . , k₁−1, where

Fₖ={\hat{R}+\hat{B}*\hat{Π}ₖ₊₁\hat{B}}⁻¹\hat{B}*\hat{Π}ₖ₊₁Â

The operators \hat{Π}ₖ, k=k₀, k₀+1, . . . , k₁−1, are the unique self-adjoint positive semi-definite operators satisfying the following Riccati difference equation

\hat{Π}ₖ=Â*[\hat{Π}ₖ₊₁−\hat{Π}ₖ₊₁\hat{B}(\hat{R}+\hat{B}*\hat{Π}ₖ₊₁\hat{B})⁻¹\hat{B}*\hat{Π}ₖ₊₁ ]Â+\hat{Q}

k=k₀, k₀+1, . . . , k₁−1, with \hat{Π}ₖ_{₁}=Ĝ.

Moreover, it follows that Ĵ(ū)= [symbol] \hat{Π}ₖ_{₀}x₀, x₀ [symbol] _{X} and that the optimal trajectory {x̅ₖ}ₖ₌ₖ_{₀}^{k}^{₁} is given by x̅ₖ₊₁=(Â−\hat{B}Fₖ) x̅ₖ. An operator \hat{Π}∈ [symbol] (X, X) is a solution to the algebraic Riccati equation (ARE) if

\hat{Π}=Â*[\hat{Π}−\hat{Π}\hat{B}(\hat{R}+\hat{B}*\hat{Π}\hat{B})⁻¹\hat{B}*\hat{Π}]Â+\hat{Q}.

The existence of a positive semi-definite self-adjoint solution to the ARE is equivalent to the existence of an admissible control for any initial condition x₀. As in the case of finite-dimensional systems, the existence of an admissible control is equivalent to saying that the system is stabilizable. On the other hand, if the operators Â, \hat{B}, \hat{Q} and \hat{R} are such that if x₀∈X and u is an admissible control for x₀, then lim_{k→∞}∥xₖ∥_{X}=0, then the system 10 is said to be detectable (we borrow the concept from finite-dimensional case) and the uniqueness of the solution to the ARE 6 is guaranteed.

Consequently, if we assume that the system 1 is both stabilizable and detectable, then there exists a unique solution \hat{Π} to the ARE 6 and a unique optimal control ū for the problem (P2) for the initial value x₀. It follows that Ĵ(ū)= [symbol] \hat{Π}x₀, x₀ [symbol] _{X} where ūₖ=−Fx̅ₖ, F=(\hat{R}+\hat{B}*\hat{Π}\hat{B})⁻¹\hat{B}*\hat{Π}Â, and the optimal trajectory {x̅ₖ}ₖ₌ₖ_{₀}^{∞} is given by x̅ₖ₊₁=(Â−\hat{B}F)x̅ₖ.

We note that it is often the case in both the finite and infinite horizon problems, there is an additional separable Hilbert space, Z, an operator D∈ [symbol] (X, Z), and a quantity to be controlled or regulated, zₖ=\hat{D}xₖ, k=k₀, k₀+1, k₀°2, . . . , in which case the positive semi-definite operator \hat{Q}∈ [symbol] (X, X) is given by \hat{Q}=\hat{D}*\hat{D}.

Finite-Dimensional Approximation (Section 11). Let X^{N}, N=1,2, . . . , be a sequence of finite-dimensional linear subspaces of a Hilbert space X and [symbol] ^{N}:X→X^{N} be the canonical orthogonal projections satisfying [symbol] ^{N}x→x for any x∈X. SHere we have an observation space Y, and a control space U that are potentially infinite-dimensional.

Assumption 1: There exist operators Â^{N}:X^{N}→X^{N}, \hat{B}^{N}:U→X^{N}, \hat{Q}^{N}:X^{N}→X^{N}, and Ĝ^{N}:X^{N}→X^{N} which satisfy Â^{N} [symbol] ^{N}x→Âx, (Â^{N})* [symbol] ^{N}x→Â*x, x∈X, \hat{B}^{N}u→\hat{B}u, u∈U, (\hat{B}^{N})* [symbol] ^{N}x→\hat{B}*x, x∈X, \hat{Q}^{N}= [symbol] ^{N}\hat{Q}= [symbol] ^{N}\hat{Q} [symbol] ^{N}, and Ĝ^{N}= [symbol] ^{N}Ĝ= [symbol] ^{N}Â [symbol] ^{N}, as N→∞.

Consider a sequence of approximating discrete-time LQR problems on the finite-time horizon [k₀, k₁]:
(P1^{N}) Choose ū^{N}∈l²(k₀, k₁−1; U) to minimize

$${\hat{J}}^{N} ( u ) = {\sum }_{k = {k}_{0}}^{{k}_{1} - 1} {〈 {\hat{Q}}^{N} {x}_{k}^{N} , {x}_{k}^{N} 〉}_{X} + {〈 \hat{R} {u}_{k} , {u}_{k} 〉}_{U} + {〈 {\hat{G}}^{N} {x}_{{k}_{1}}^{N} , {x}_{{k}_{1}}^{N} 〉}_{X}$$

$$where$$

$${x}_{k + 1}^{N} = {\hat{A}}^{N} {x}_{k}^{N} + {\hat{B}}^{N} {u}_{k} , {x}_{{k}_{0}}^{N} = {x}_{0}^{N} = {𝒫}^{N} {x}_{0} , k \geq {k}_{0}$$

The results concerning the existence and uniqueness of the solution to the discrete-time LQR problem on the finite-time horizon in a general Hilbert space, (P1), outlined in the previous section can be applied to each of the approximating finite-dimensional problems (P1^{N}). The formulas characterizing the solution to problem (P1^{N}) have the same form as those for problem (P1).

The fundamental convergence result is given by the following theorem.

Theorem 1: Let ū^{N} and ū be the unique solutions to the approximation problem (P1^{N}) and the original problem (P1), respectively. x̅^{N} and x̅ are the corresponding optimal trajectories. Ĵ^{N}, \hat{Π}ₖ^{N}, and Fₖ^{N} are from (P1^{N}), and Ĵ, \hat{Π}ₖ, and Fₖ are defined as before in (P1). Then if Assumption 1 holds, we have

lim_{N→∞}|ū^{N}−ū|ₗ_{²}=0, (i)

lim_{N→∞}|x̅^{N}−x̅|ₗ_{²}=0, (ii)

lim_{N→∞}|Ĵ^{N}(ū^{N})−\hat{J}(u̅)|=0, (iii)

lim_{N→∞}|\hat{Π}ₖ^{N} [symbol] ^{N}x−\hat{Π}ₖx|_{X}=0, x∈X, k₀≤k≤k₁, (iv)

lim_{N→∞}|Fₖ^{N} [symbol] ^{N}x−Fₖx|=0, x∈X, k₀≤k≤k₁−1, (v)

where the l² inner product and corresponding norm is defined by [symbol] x, y [symbol] ₗ_{²}=Σₖ₌ₖ_{₀}^{k}^{₁} [symbol] kₖ, yₖ [symbol] _{H} for any x and y in l² (k₀, k₁; H), and any Hilbert space H.

Note that if U is m-dimensional (i.e. U= [symbol] ^{m}) and F∈ [symbol] (X, U), then by the Riesz Representation Theorem there exists ƒ∈xᵢ₌₁^{m}X, the so-called functional gains corresponding to F, such that u=Fx=[ [symbol] ƒ₁, x [symbol] , . . . , [symbol] ƒₘ,x [symbol] ]^{T}, with [symbol] ⋅, ⋅ [symbol] denoting the X inner product.

Remark 1: If in addition we have that the control or input space, U, is finite-dimensional with dimension m, it then follows that Fₖ^{N} [symbol] ^{N}→Fₖ, k₀≤k≤k₁−1, in norm (i.e. in [symbol] (X, U)) and the m-dimensional functional gains corresponding to F^{N} [symbol] ^{N} and Fₖ, k₀≤k≤k₁−1, ƒ^{N} and ƒ, respectively, satisfy ƒ^{N}→ƒ in l²(k₀, k₁−1; xᵢ₌₁^{m}X).

Remark 2: If the control or input space is finite-dimensional with dimension m and Assumption 1 holds with the exception that (Â^{N})* [symbol] ^{N}x→Â*x, x∈X (i.e. only weak rather than strong convergence of the adjoint), it then follows that \hat{Π}ₖ^{N} [symbol] ^{N}x→\hat{Π}ₖx, x∈X, k₀≤k≤k₁, Fₖ^{N} [symbol] ^{N}x→Fₖx, x∈X, k₀≤k≤K₁−1, and ƒ^{N}→ƒ in l²(k₀, k₁−1; xᵢ₌₁^{m}X)

Now consider a sequence of approximating discrete-time LQR problems on the infinite-time horizon [k₀, ∞):
(P2^{N}) Choose ū^{N}∈l²(k₀, ∞; U) to minimize

$${\hat{J}}^{N} ( u ) = {\sum }_{k = {k}_{0}}^{∞} {〈 \hat{Q} {x}_{k}^{N} , {x}_{k}^{N} 〉}_{X} + {〈 \hat{R} {u}_{k} , {u}_{k} 〉}_{0}$$

for the same system 7).

To guarantee the solvability of (P2^{N}), we need to assume the solvability of the approximating finite-dimensional AREs, i.e. for each N, there exists exactly one positive semi-definite self-adjoint solution to the approximation ARE.

As in the infinite-dimensional case, let F^{N}=(\hat{R}+(\hat{B}^{N})*\hat{Π}^{N}\hat{B}^{N})⁻¹(\hat{B}^{N})*\hat{Π}^{N}Â^{N}, Ŝ^{N}=Â^{N}−\hat{B}^{N}F^{N}

where \hat{Π}^{N} is the unique positive semi-definite self-adjoint solution to the approximating ARE assumed to exist. We then have the following convergence theorem.

Theorem 2: Under Assumption 1 if \hat{Π}^{N} [symbol] ^{N} converges strongly to some bounded linear operator \hat{Π}, then \hat{Π} is a positive semi-definite self-adjoint solution to the original ARE 6, F^{N} [symbol] ^{N} converges strongly to F and Ŝ^{N} [symbol] ^{N} converges strongly to Ŝ, where F is defined in the original infinite-dimensional problem (P2) and S=Â−\hat{B}F.

Modifications to Theorem 1 analogous to those given in Remark 1 and Remark 2 apply to Theorem 2 as well. We have the following result.

Theorem 3: Under Assumption 1 suppose that there exists positive constants M and r, independent of N, with r<1, such that

\hat{Π}^{N}≤M, N=1,2, . . . ,

|(Ŝ^{N})^{t}|≤Mr^{t}, t=1,2, . . . , N=1,2, . . . ,

where \hat{Π}^{N} is the unique positive semi-definite self-adjoint solution to the approximating ARE assumed to exist. Then a positive semidefinite self-adjoint solution \hat{Π} to 6 exists, and \hat{Π}^{N} [symbol] ^{N}→\hat{Π} strongly as N→∞. If there exists a positive m, independent of N, such that |\hat{Q}^{N}≥m, N=1,2, . . . , then this implies the existence of an r less than one and independent of N for which the above equation holds.

Finally we note that it is also possible to fully discretize the problems (P1) and (P2) with the introduction of a sequence of finite-dimensional approximating subspaces, {U^{M}} of the in general infinite-dimensional input or control Hilbert space U and obtain a doubling indexed sequence of approximating LQR problems on either the finite or infinite time horizon Straight forward extensions of the theorems presented above can be proven which establish analogous convergence results as N, M→∞.

The LQG Observer and Compensator (Section 12). The LQG compensator is based on combining the LQR theory described above with a Kalman filter state estimator or observer. The general theory for discrete-time systems in Hilbert space together with a finite-dimensional approximation and convergence results can be found. The observer or state estimator takes the form

\tilde{x}ₖ₊₁=Âxₖ+\hat{B}uₖ+\tilde{L}ₖ(yₖ−Ĉ\tilde{x}ₖ), \tilde{x}ₖ_{₀}=x₀, k≥k₀

where x₀∈X is arbitrary, the operators \tilde{L}ₖ∈ [symbol] (Y, X) are given by \tilde{L}ₖ=Â\tilde{Π}ₖĈ*{\tilde{R}+Ĉ\tilde{Π}ₖĈ*}, with the operators \tilde{Π}ₖ, k=k₀, k₀+1, . . . given by the recurrence

\tilde{Π}ₖ₊₁=Â[\tilde{Π}ₖ−\tilde{Π}ₖ\hat{C}*(\tilde{R}+Ĉ\tilde{Π}ₖĈ*)⁻¹Ĉ\tilde{Π}ₖ]Â*+\tilde{Q}, (10)

k=k₀, k₀+1, . . . , with \tilde{Π}ₖ_{₀}=0, \tilde{Q}=σ_{Q}²\hat{B}₁\hat{B}*₁, and \tilde{R}=σ_{R}²Ĉ₁Ĉ*₁. The optimal LQG compensator or controller is then given by ũₖ=−Fₖ\tilde{x}ₖ, where the feedback operators {Fₖ} above.

The steady state form is given by Lₖ=L where \tilde{L}∈ [symbol] (Y, X) is given by: \tilde{L}=Â\tilde{Π}Ĉ*{\tilde{R}+Ĉ\tilde{Π}Ĉ*}⁻¹, with the operator \tilde{Π} a positive semi-definite self-adjoint solution, if it exists, to the ARE given by

\tilde{Π}=Â[\tilde{Π}−\tilde{Π}Ĉ*(\tilde{R}+Ĉ\tilde{Π}Ĉ*)⁻¹Ĉ\tilde{Π}]Â*+\tilde{Q}

Note that if the output space Y is m-dimensional, then the optimal observer gains \tilde{L}ₖ or \tilde{L} can be represented by an m-dimensional row vector ƒₖ or ƒ of elements in X. These are referred to as the optimal functional observer gains.

In light of the duality between the LQR control and the LQG observer problems, existence and uniqueness results for solutions to the ARE are analogous to those given for the LQR ARE. Finite-dimensional approximation and convergence results for the observer/compensator are also analogous to the LQR theory presented above. Indeed, if in addition to Assumption 1 we have that there exist operators Ĉ^{N}∈ [symbol] (X^{N}, Y) and positive semi-definite self-adjoint operators \tilde{Q}^{N}∈ [symbol] (X^{N}, X^{N}) such that Ĉ^{N} [symbol] ^{N}x→Ĉx, x∈X, (Ĉ^{N})*y→Ĉ*y, y∈Y and \tilde{Q}^{N} [symbol] ^{N}x→\tilde{Q}x, x∈X, as N→∞, we have that the solutions to the finite-dimensional approximating observer Riccati equations converge strongly to the solutions to the infinite-dimensional Riccati equations, and that the approximating optimal observer gain operators converge strongly to their infinite-dimensional counterparts. In the case that the output space is finite-dimensional, the approximating optimal functional observer gains converge in norm as well.

We note that in the steady state case, the state transition operator for the closed loop plant/compensator system is given

$$S = [ \begin{matrix}\hat{A} & - \hat{B} F \\ \tilde{L} \hat{C} & \hat{A} - \hat{B} F - \tilde{L} C .\end{matrix} ]$$

with the (closed-loop) spectrum of S given by σ(S)=σ(Â−\hat{B}F)∪σ(Â−\tilde{L}Ĉ).

### Abstract Parabolic Systems With Random Parameters (Section 13).

Abstract Parabolic Systems (Section 13. A). Let V and H be Hilbert spaces with V [symbol] H, i.e. V is continuously and densely embedded in H. Then the Gelfand triple V [symbol] H [symbol] V* is obtained by identifying H with its dual H*. Define the inner product on H by [symbol] ⋅, ⋅ [symbol] _{H} and the norms on H and V by |⋅|_{H}, ∥⋅∥_{V}, respectively. Define a sesquilinear form a(⋅, ⋅):V×V→ [symbol] which satisfies the following properties.

Assumption 2: (Boundedness) There exists a constant α₀>0 such that for each φ, ψ∈V, we have

|a(φ, ψ)|≤α₀∥φ∥_{V}∥ψ∥_{V}.

Assumption 3: (Coercivity) There exist constants λ₀∈ [symbol] and μ₀>0 such that for each φ∈V, we have

a(φ, φ)+λ₀|φ|_{H}²≥μ₀∥φ∥_{V}².

Now we consider the following parabolic system written in the weak form:

[symbol]

\dot{x}, ψ [symbol] _{V*,V}+a(x, ψ)= [symbol] Bu, ψ [symbol] _{V*,V}, ψ, ∈V, x(0)=x₀

where x₀∈H, u∈L²([0, T], U) is an input to the system, and B:U→V* is a bounded linear operator.

It can be shown that the equation has a unique solution in the set:

{ψ:ψ∈L²([0, T], V), \dot{ψ}∈L²([0, T], V*)}⊆C([0, T], H).

Under these assumptions, a(⋅, ⋅) defines a bounded linear operator A:V→V* such that

−a(φ, ψ)= [symbol] Aφ, ψ [symbol] _{V*,V}

where φ, ψ∈V.

Furthermore, it can be shown that A restricted on the set

Dom (A)={φ∈V:Aφ∈H}

is the infinitesimal generator of a holomorphic or analytic semigroup of bounded linear operators on H, {T(t):t≥0}. Moreover, this semigroup can be restricted to be a holomorphic semigroup on V and extended to be a holomorphic semigroup on V* by appropriately restricting or extending the domain. Dom (A), of the operator A.

It follows that the system can be rewritten in state space form as the evolution system with time-invariant operators A and B:

\dot{x}(t)=Ax(t)+Bu(t), x(0)=x₀.

Systems with Random Parameters (Section 13. B). Now we summarize the key idea from the framework and consider an abstract parabolic system with random parameters satisfying some known distribution. Assume q∈Q, where the set of admissible parameters, Q is a compact subset of the finite-dimensional Euclidean space whose dimension is p, and is compact with respect to some metric d_{Q}. For each q∈Q, we require that besides satisfying Assumption 2 and 3, the sesquilinear form a(q; ⋅⋅):V×V→ [symbol] also satisfies:

Assumption 4: (Continuity) For q, \tilde{q}∈Q, we have for all φ, ψ∈V,

|a(q; φ, ψ)−a(\tilde{q}; φ, ψ)|≤d_{Q}(q, \tilde{q})∥φ∥∥ψ∥,

where d_{Q}(⋅, ⋅) denotes any p-metric on [symbol] ^{p}. It is assumed that all of the constants, α₀, λ₀, and μ₀ do not depend on q, for q∈Q.

In addition, it may also sometimes be required that the inner product on the space H depend upon q∈Q. Let this space be denoted by H_{q}={H, [symbol] ⋅, ⋅ [symbol] _{q}, |⋅|_{q}} and that the following assumption be satisfied.

Assumption 5: (H-Continuity) For q, \tilde{q}∈Q, we have for all φ, ψ∈H,

[symbol]

φ, ψ [symbol] _{q}− [symbol] φ, ψ [symbol] _{\tilde{q}}|≤d_{Q}(q, \tilde{q})|φ|_{H}|ψ|_{H}

and that the identity map from V into H_{q} be uniformly bounded for q∈Q.

We formulate a population model by treating the parameter q as a random vector q, and assume that its support is xᵢ₌₁^{p}[aᵢ, bᵢ], where aᵢ, bᵢ are real numbers since we have assumed that the set of admissible parameters Q is a compact subset of a finite Euclidean space, i.e. −∞<aᵢ<bᵢ<∞ on for all i=1,2, . . . , p. Typically, the distribution of q will depend on parameters in some parameter set Θ⊂ [symbol] ^{r} for some r which is closed and bounded. That is, we assume that the distribution of q is given by a known measure π=π(ρ)=π(\vec{a}, \vec{b}, \vec{θ}), where ρ=(\vec{a}, \vec{b}, \vec{θ}) with \vec{a}=[aᵢ]ᵢ₋₁^{p}, \vec{b}=[bᵢ]ᵢ₌₁^{p}, and \vec{a}∈Θ. It will typically be the case that the population model is determined by fitting the parameters ρ=(\vec{a}, \vec{b}, \vec{θ}) to population data.

Define the Bochner spaces [symbol] =L_{π}²(Q; V), [symbol] =L_{π}²(Q; H_{q}) corresponding to the measure π. Then the assumptions on the spaces V and H guarantee that the spaces [symbol] and [symbol] form the Gelfand triple [symbol] . Here [symbol] has been identified with its dual [symbol] =L_{π}²(Q; H*_{q}).

We then define the i-averaged sesquilinear form a(⋅; ⋅): [symbol] × [symbol] → [symbol] by

a(φ, ψ)=∫_{Q}a(q; φ(q), ψ(q))dπ(q)= [symbol] _{π}[a(q; φ(q), ψ(q))]

where φ, ψ∈ [symbol] . Assumptions 23 and 4 guarantee that this integral is well defined on q.

We can easily check the boundedness and coercivity of a(⋅, ⋅) by using Assumption 24 and the Cauchy-Schwartz Inequality.

Therefore, we can use this sesquilinear form to define a bounded linear map [symbol] → [symbol] by

[symbol]

[symbol]

φ, ψ [symbol] [symbol] =−a(φ, ψ), φ, ψ∈

[symbol]

which when appropriately restricted or extended is the infinitesimal generator of analytic semigroups of bounded linear operators [symbol] (t), t≥0 on [symbol] and [symbol] .

We next consider a nonhomogeneous parabolic system with random parameters written in the weak form:

[symbol]

\dot{x}, ψ [symbol] _{V*,V}+a(q; x, ψ)= [symbol] B(q)u, ψ [symbol] _{V*,V}ψ∈V

where B(q):U→V* is a bounded linear operator defined on a Hilbert space of feasible inputs, U, and u∈L²([0, ∞), L_{π}²(Q; U)). Then let [symbol] be a closed subspace of the Bochner space L_{π}²(Q; U) and define the operator [symbol] → [symbol] by

[symbol]

[symbol]

u, ψ [symbol] [symbol] =∫_{Q} [symbol] B(q)u(q), ψ(q) [symbol] _{V*,V}dπ(q)

where u∈ [symbol] , ψ∈ [symbol] .

In light of 13 and 16 , as in the deterministic case, we can write the population model corresponding to (15) in weak (with respect to both η∈(0,1) and q∈Q) form as

[symbol]

\dot{x}, ψ [symbol] [symbol] +a(x, ψ)= [symbol] [symbol] u, ψ [symbol] [symbol] , ψ∈ [symbol] ,

and then, in state space form as

\dot{x}(t)= [symbol] x(t)+ [symbol] u(t), x(0)=x₀,

where u∈L²([0, ∞), [symbol] ) It is shown that the solutions agree almost surely or π−a, eq∈Q. It is interesting to note that in this way, the random parameters are treated like additional spatial variables and in particular the resulting weak form does not involve any derivatives with respect to these variables. More to the point, the resulting dynamical system is now effectively deterministic and abstract parabolic and thus amenable to the treatment for standard abstract parabolic systems discussed previously in subsection V−A

From linear semigroup theory, the mild solution is then given by:

x(t)= [symbol] (t)x₀+∫₀^{t} [symbol] (t−s) [symbol] u(s)ds, t≥0

Let τ denote the length of the sampling interval, and consider zeroorder hold inputs of the form u(t)=uₖ, for t∈[kτ, (k+1)τ), k=0,1,2, . . . . . If we then define xₖ=x(kτ), k=0,1,2, . . . and [symbol] ∈ [symbol] ), and [symbol] ∈ [symbol] respectively by [symbol] = [symbol] (τ) and [symbol] =∫₀^{τ} [symbol] (s) [symbol] ds, We obtain the discrete-time dynamical system given by:

xₖ₊₁= [symbol] xₖ+ [symbol] uₖx(0)=x₀.

We note that if the operator [symbol] given in \sqrt{14} is invertible (for example if λ₀=0 in Assumption 3, it follows that [symbol] =∫₀^{τ}(s) [symbol] ds=( [symbol] −I) [symbol] ⁻¹ [symbol] = [symbol] ⁻¹( [symbol] −I) [symbol] .

Finally we note that if there is an output or observation operator C(q)∈ [symbol] (H_{q}, [symbol] ) (or L(V, [symbol] )), where [symbol] denotes the observation Hilbert space, let [symbol] = [symbol] and define [symbol] ∈ [symbol] ( [symbol] ) (or [symbol] ( [symbol] )) by [symbol] v=∫_{Q}C(q)v(q)dπ(q). Then the system 20 can be augmented with the output equation yₖ= [symbol] xₖ, k=0,1,2, . . .

Finite-Dimensional Approximation and Convergence (Section 13. C.). We consider a Galerkin approximation based on the weak form). Let N be a positive integer. For each N, let [symbol] ^{N} be a finite-dimensional subspace of [symbol] , satisfying [symbol] ^{N}x→x, for x∈ [symbol] , where [symbol] ^{N} is the orthogonal projection of [symbol] onto [symbol] ^{N}.

Now we define the operators [symbol] ^{N} on [symbol] ^{N} by essentially restricting the form a to the subspace [symbol] ^{N}× [symbol] ^{N} of the space [symbol] × [symbol] . To be more specific, we have

$$\begin{matrix}{〈 {𝒜}^{N} {φ}^{N} , {ψ}^{N} 〉}_{𝒱}^{N} , {𝒱}^{N} = - a ( {φ}^{N} , {ψ}^{N} ) \\ = - {\int }_{Q} a ( q ; {φ}^{N} ( q ) , {ψ}^{N} ( q ) ) d π ( q ) ,\end{matrix}$$

where φ^{N}, ψ^{N}∈ [symbol] ^{N}

Obviously, since [symbol] ^{N} is a linear operator on a finite-dimensional space, it is the infinitesimal generator of a uniformly continuous semigroup [symbol] ^{N}(t)= [symbol] for t≥0. So we can define [symbol] ^{N}∈ [symbol] ( [symbol] ^{N}, [symbol] ^{N}) by Â^{N}= [symbol] ^{N}(τ)= [symbol] τ

We use a variational corollary of Trotter-Kato theorem to obtain the convergence of semigroup. Thereby, we obtain the convergence of operator Â^{N}.

Theorem 4: Assume that the Assumptions 24 are satisfied. Then for each x∈ [symbol] , [symbol] ^{N}(t) [symbol] ^{N}x→ [symbol] (t)x in the [symbol] norm for t>0 uniformly in ton compact sub intervals.

Remark 3: The Trotter-Kato theorem requires the following assumption: For each x∈ [symbol] , there exists x^{N}∈ [symbol] ^{N} such that ∥x−x^{N} [symbol] →0. However, we note that this assumption is actually equivalent to the strong convergence of orthogonal projection [symbol] ^{N} of [symbol] onto [symbol] ^{N}, i.e. for any x∈ [symbol] , [symbol] ^{N}x→x as N→∞. Indeed, for any x∈ [symbol] satisfying the assumption in [4], we have ∥ [symbol] ^{N}x−x [symbol] ≤∥x−x^{N} [symbol] →0. On the other hand, for any x∈ [symbol] satisfying P̅^{N}x→x as N→∞, take x^{N}= [symbol] ^{N}x∈ [symbol] ^{N}then by [symbol] ^{N}→I strongly, we have ∥x−x^{N}∥ [symbol] =∥x− [symbol] ^{N}x∥ [symbol] →0.

From the definition [symbol] = [symbol] (τ) and [symbol] ^{N}= [symbol] ^{N}(τ), we immediately get that ( [symbol] ^{N} [symbol] ^{N}→ [symbol] strongly in [symbol] as N→∞.

Weak convergence of the adjoint of [symbol] ^{N}, ( [symbol] ^{N})*, which is also a bounded operator on [symbol] ^{N}, then immediately follows.

Corollary 4.1: Let ( [symbol] ^{N})*, Â* and [symbol] ^{N} be defined as before. Then ( [symbol] ^{N})* [symbol] ^{N}→ [symbol] * (i.e. weakly) in [symbol] .

Proof. For any φ, ψ in [symbol] , we have

$$\begin{matrix}〈 ( {\hat{𝒜}}^{N} ) * {𝒫}^{N} φ , ψ 〉 = 〈 {𝒫}^{N} ( {\hat{𝒜}}^{N} ) * {𝒫}^{N} φ , ψ 〉 \\ = 〈 ( {\hat{𝒜}}^{N} ) * {𝒫}^{N} φ , {𝒫}^{N} ψ 〉 = 〈 {𝒫}^{N} φ , {\hat{𝒜}}^{N} {𝒫}^{N} φ 〉 \\ = 〈 φ , {𝒫}^{N} {\hat{𝒜}}^{N} {𝒫}^{N} φ 〉 = 〈 φ , {\hat{𝒜}}^{N} {𝒫}^{N} ψ 〉 ,\end{matrix}$$

where the inner product in the above calculation is the [symbol] inner product. Since [symbol] ^{N} [symbol] ^{N}→\hat{d} strongly in [symbol] as N→∞, we have

[symbol]

( [symbol] ^{N})* [symbol] ^{N}φ, ψ [symbol] → [symbol] φ, [symbol] ψ [symbol] = [symbol] [symbol] *φ, ψ [symbol] .

We note that the Trotter-Kato theorem can also be used to argue that [symbol] ^{N}(t) [symbol] ^{N}x→ [symbol] ^{N}(t)x in the [symbol] norm for t>0, uniformly in t on compact sub intervals. Moreover, since the operator d is regularly dissipative, the same arguments can also be used to argue that [symbol] ^{N}(t)* [symbol] ^{N}x→ [symbol] (t)*x in the [symbol] nom for t>0. uniformly in t on compact sub intervals and consequently that both [symbol] ^{N} and ( [symbol] ^{N})* converge to [symbol] and ( [symbol] )*, respectively, strongly in [symbol] . However, it is worth noting that for some problems of interest the observation operator is bounded in [symbol] but unbounded in [symbol] , and in such a case, one may want to apply the LQR theory developed earlier in Sections II and III in [symbol] rather than in [symbol] . In this event arguing strong convergence of the adjoint may be difficult or simply not true. in which case, the weaker convergence results for the approximating solutions to the LQR problem will have to suffice.

An Example: A Random Parabolic ODE/PDF Hybrid System with Coupling on the Boundary of the Spatial Domain (Section 14)

We consider the design of an LQG control or regulator for a clamping experiment involving the intravenous infusion of ethanol with observations provided by a transdermal alcohol biosensor. The dynamical model takes the form of a hybrid, semi-linear, ODE/PDE reaction diffusion equation. The transdermal transport of ethanol through the epidermal layer of the skin is modeled by a one-dimensional diffusion equation which is coupled via Dirichlet boundary conditions to two well-mixed compartments, one representing the blood and the other the transdermal alcohol biosensor. The inflow to the two compartments is proportional to the flux at the boundary of the epidermal layer of the skin. Aside from the relatively small amount of ethanol excreted from the body through urine, tears, breast milk, sweat and perspiration, the primary mechanism by which ethanol is processed out of the body is via a reaction that takes place in the liver and which is catalyzed by a group of enzymes known as alcohol dehydrogenase (ADH). In the transdermal alcohol biosensor, the ethanol is consumed in an oxidation-reduction reaction wherein each molecule of ethanol produces four electrons. The resulting current is measured with the measurement being bench calibrated with a source of ethanol vapor with known concentration. The enzyme catalyzed reaction in the blood compartment (liver) is modeled Michaelis-Menten term which exhibits first-order kinetics at low concentrations and zero-order kinetics at higher concentrations once saturation is achieved. In addition, since the values of the parameters which appear in the model for an individual subject will in all likelihood be unknown and un-measurable, we will consider the parameters to be random with distribution that has previously been fit to cohort from an appropriately stratified population. Consequently, the resulting control problem is one in which the process is to be regulated for an individual based on a population model.

Problem Formulation (Section 11. A.). The underlying dynamical system as described in the previous paragraph takes the following form:

$$\begin{matrix}\frac{\partial \tilde{x}}{\partial t} ( t , η ) = α \frac{{\partial }^{2} \tilde{x}}{\partial {η}^{2}} ( t , η ) , t > 0 , η ∈ ( 0 , 1 ) , \\ \frac{d \tilde{w}}{dt} ( t ) = β \frac{\partial \tilde{x}}{\partial η} ( t , 0 ) - γ \overline{w} ( t ) + {ω}_{1} ( t ) , t > 0 , \\ \frac{d \tilde{v}}{dt} ( t ) = - δ + \frac{\partial \tilde{x}}{\partial η} ( t , 1 ) - \frac{K \tilde{v} ( t )}{M + \tilde{𝓋} ( t )} + b \tilde{u} ( t ) + {ω}_{2} ( t ) , t > 0 ,\end{matrix}$$

with boundary conditions, controlled variable and observation:

\tilde{x}(t, 0)=\tilde{w}(t), \tilde{x}(t, 1)=\tilde{v}(t), t>0,

\tilde{z}(t)=\tilde{v}(t), \tilde{y}(t)=\tilde{w}(t)+ζ(t), t>0,

respectively, and initial conditions:

\tilde{v}(0, η)=φ₀(η), η∈(0, 1), \tilde{w}(0)=θ₀, \tilde{v}(0)=ξ₀,

where the parameters appearing in the model equations 21-23, α, β, γ, δ, M, K, and b are all assumed to be positive, and the initial conditions φ₀, θ₀, and ξ₀ are all assumed to be nonnegative. In the above system x̅(t, η) is the concentration of ethanol at time t≥0 and depth η∈[0,1] in the epidermal layer, \tilde{w}(t) is the concentration of ethanol in the transdermal alcohol biosensor vapor collection chamber at time t≥0, \tilde{v}(t) is the concentration of ethanol in the blood at time t≥0, and ũ(t) is the concentration of ethanol in the infused intravenous solution at time t≥0. In addition, ω₁, ω₂, and ζ denote uncorrelated, zero-mean, stationary, Gaussian white noise processes with variances σ₁², σ₂², and σ², respectively. We note that without loss of generality we have normalized the thickness of the epidermal layer to be one. Also, it is possible to include random noise in the diffusion equation using one of the available treatments of stochastic processes in infinite-dimensional space. However, in the interest of clarity, since this is not the central focus of this research, we have decided to omit this.

If the desired clamped blood alcohol level is \tilde{v}(t)=\tilde{v}₀, then an equilibrium solution to the system 21 is given by

$$\tilde{x} ( t , η ) = {\tilde{x}}_{0} ( η ) = \frac{γ {v}_{0}}{γ + β} η + \frac{β {v}_{0}}{γ + β} ,$$

$$\tilde{w} ( t ) = {\tilde{w}}_{0} = \frac{β {v}_{0}}{γ + β} , \tilde{v} ( t ) = {\tilde{v}}_{0} , and$$

$$\tilde{u} ( t ) = {\tilde{u}}_{0} = \frac{δγ {\tilde{v}}_{0}}{b ( γ + β )} + \frac{K {\tilde{v}}_{0}}{b ( M + {v}_{0} )} .$$

To formulate the regulator problem, we linearize about a clamped operating regime, \tilde{x}₀, \tilde{w}₀, \tilde{v}₀, and ũ₀, by writing \tilde{x}=\tilde{x}₀+x, \tilde{w}=\tilde{w}₀+w, \tilde{v}=\tilde{v}₀+v, and ũ=ũ₀+u and obtain the linearized system for x, w, v, and u given by:

$$\frac{\partial x}{\partial t} ( t , η ) = {q}_{1} \frac{{\partial }^{2} x}{\partial {η}^{2}} ( t , η ) , t > 0 , η ∈ ( 0 , 1 )$$

$$\frac{dw}{dt} ( t ) = {q}_{3} \frac{\partial x}{\partial η} ( t , 0 ) - {q}_{4} w ( t ) + {ω}_{1} ( t ) , t > 0$$

$$\frac{dv}{dt} ( t ) = - {q}_{5} \frac{\partial x}{\partial η} ( t , 1 ) - {q}_{6} v ( t ) + {q}_{2} u ( t ) + {ω}_{2} ( t ) , t > 0$$

with boundary conditions, controlled variable and observation

x(t,0)=w(t), x(t, 1)=v(t), t>0,

z(t)=v(t), y(t)=w(t)+ζ(t), t>0,

respectively, where in the equations the parameters q₁=α, q₂=b, q₃=β, q₄=γ, q₅=δ, and

$${q}_{6} = \frac{KM}{{( M + {\overline{v}}_{0} )}^{2}}$$

are all positive. The state of the system is given by the triple (w, v, x) and the control objective is to determine an output feedback law for u that drives v to zero based on the observation of w, with the caveat that we only know the distribution of the parameters q=(q₁, q₂, q₃, q₄, q₅, q₆) in the subject cohort of interest.

We reformulate the system as an abstract parabolic system in a Gelfand triple of Hilbert spaces. Let Q be a compact subset of the positive orthant of [symbol] ⁶, let H= [symbol] ²×L²(0, 1) be endowed with the standard inner product and norm and for q∈Q let H_{q}= [symbol] ²×L²(0, 1) with the inner product

$${〈 ( θ , ξ , φ ) , ( \overline{θ} , \overline{ξ} , \overline{φ} ) 〉}_{q} = \frac{{q}_{1}}{{q}_{3}} θ \overline{θ} + \frac{{q}_{1}}{{q}_{5}} ξ \overline{ξ} + {\int }_{0}^{1} φ ( η ) \overline{φ} ( η ) d η$$

Let V be the Hilbert space

$$V = \{ ( θ , ξ , φ ) ∈ H : φ ∈ {H}^{1} ( 0 , 1 ) , θ = φ ( 0 ) , ξ = φ ( 1 ) \}$$

$${{〈 ( φ ( 0 ) , φ ( 1 ) , φ ) , ( \overline{φ} ( 0 ) , \overline{φ} ( 1 ) , \overline{φ} ) 〉}_{V} = φ ( 0 ) \overline{φ} ( 0 ) + φ ( 1 ) \overline{φ} ( 1 ) + 〈 φ , \overline{φ} )}_{{H}^{1} ( 0 , 1 )}$$

where [symbol] ⋅, ⋅ [symbol] _{H}_{¹}_{(0,1)} denotes the standard inner product on H¹(0,1). Standard arguments yield the dense and continuous embeddings V [symbol] H_{q} [symbol] V* and that Assumption 5 is satisfied. Define the bilinear form a(q; ⋅, ⋅):V×V→ [symbol] by

$$a ( q ; ( φ ( 0 ) , φ ( 1 ) , φ ) , ( \overline{φ} ( 0 ) , \overline{φ} ( 1 ) , \overline{φ} ) ) = \frac{{q}_{1} {q}_{4}}{{q}_{3}} φ ( 0 ) \ddot{φ} ( 0 ) + \frac{{q}_{1} {q}_{6}}{{q}_{5}} φ ( 1 ) \ddot{φ} ( 1 ) + {q}_{1} {\int }_{0}^{1} {φ}^{′} ( η ) {\ddot{φ}}^{′} ( η ) d η$$

Our continuity and compactness assumptions and standard and straight forward calculations yield that the form a(q; ⋅, ⋅) satisfies Assumptions 25 and 4 with all relevant constants appearing in those assumptions independent of q∈Q.

Let A(q):Dom (A(q)⊂H→H be given by:

[symbol]

A(q)\hat{φ}, \hat{ψ} [symbol] _{V*,V}=−a(q; \hat{φ}, \hat{ψ})

for \hat{φ}∈Dom (A(q)), and \hat{ψ}∈V, where

Dom (A(q))={\hat{φ}=(φ(0), φ(1), φ)∈V:φ∈H²(0,1)}

is independent of q∈Q. Moreover, it is not difficult to show that for \hat{φ}=(φ(0), φ(1), φ)∈Dom (A(q)) we have

$$\begin{matrix}A ( q ) \hat{φ} = A ( q ) ( φ ( 0 ) , φ ( 1 ) , φ ) \\ = ( {q}_{3} {φ}^{′} ( 0 ) - {q}_{4} ( 0 ) , - {q}_{5} {φ}^{′} ( 1 ) - {q}_{6} φ ( 1 ) , {q}_{1} {φ}^{″} ) ,\end{matrix}$$

and that the operator A(q) is densely defined on H_{q}, regularly dissipative and self-adjoint. Consequently A(q) is the infinitesimal generator of a uniformly exponentially stable, self-adjoint, analytic semigroup of bounded linear operators, {T(q; t):t≥0}, on H_{q}. The state variable to be controlled or regulated is v and consequently we define the controlled variable operator D∈ [symbol] (H_{q}, [symbol] ) by D(θ, ξ, φ)=ξ. The observed state variable is w and therefore the observation or output operator C∈ [symbol] (H_{q}, [symbol] ) is given by C(θ, ξ, φ)=θ. For q∈Q, the input operator B(q)∈ [symbol] ( [symbol] , H_{q}) is given by B(Q)u=(0, q₂u, 0), and the random noise influence operator B₁∈ [symbol] ( [symbol] ², H_{q}) by B₁ω=(ω₁, ω₂, 0). In this example, we have U=Y=Z= [symbol] , all of which are clearly finite dimensional. For the finite-time horizon problem if a terminal penalty is to be included, the operator G∈ [symbol] (H_{q}, H_{q}) would most likely be chosen to be G=ρD*D, for some nonnegative weight ρ. We assume zero-order hold input and random noise with sampling time τ>0, and we consider the quadratic performance index

$$\hat{J} ( u ) = 𝔼 \{ {\sum }_{k = {k}_{0}}^{{k}_{1} - 1} {〈 \hat{Q} {x}_{k} , {x}_{k} 〉}_{{ℋ}_{q}} + \hat{r} {u}_{k}^{2} + {〈 \hat{G} {x}_{{k}_{1}} , {x}_{{k}_{1}} 〉}_{{H}_{q}} \}$$

where \hat{r}>0, k₁ can be either finite or infinite (in the latter case, ρ=0), and xₖ is given by the recurrence xₖ₊₁=Â(q)xₖ+\hat{B}(q) uₖ+\hat{B}₁(q)ω(kτ), x₀=(w(0), v(0), x(0, ⋅)), with ω(t)=[ω₁(t), ω₂(t)]^{T}, Â(q)=T(q; τ)∈ [symbol] (H_{q}, H_{q}) and, recalling that A(q) is coercive, that \hat{B}(q)=A(q)⁻¹(Â(q)−I)B(q)∈ [symbol] ( [symbol] , H_{q})=H_{q} with \hat{B}(q)∈Dom (A(q)), and \hat{B}₁(q)=A(q)⁻¹(Â(q)−I)B₁∈ [symbol] ( [symbol] ², H_{q})=H_{q}×H_{q} with \hat{B}₁(q)∈Dom (A(q)×Dom (A(q). Furthermore, it follows that C=C and \hat{D}=D, \hat{Q}=\hat{D}*\hat{D}∈ [symbol] (H_{q}, H_{q}), Ĝ=ρ\hat{D}*\hat{D}∈ [symbol] (H_{q}, H_{q}), and \hat{R}=\hat{r}.

In the observer or estimator, the state covariance operator and output covariance matrix are given by \tilde{Q}(q)=\hat{B}₁(q)Σ\hat{B}₁(q)*∈ [symbol] (H_{q}, H_{q}) where Σ=diag (σ₁², σ₂²)∈ [symbol] ^{2×2}, and \tilde{R}=σ²∈ [symbol] , respectively.

Now let q be a random vector with support Q and distribution described by the probability measure π with all functions involving qπ-measurable. Let [symbol] be the Bochner space [symbol] =L_{π}²(Q; V) and let [symbol] * be its dual. Let [symbol] =L_{π}²(Q; H_{q}), and identify the Hilbert space H [symbol] with its dual to obtain the Gelfand triple [symbol] [symbol] [symbol] [symbol] [symbol] . Define the bilinear form a(⋅, ⋅) on [symbol] × [symbol] and the operator [symbol] ∈ [symbol] ( [symbol] , [symbol] *) by:

$$\begin{matrix}a ( \hat{φ} , \hat{ψ} ) = {𝔼}_{π} \{ a ( q ; \hat{φ} ( q ) , \hat{ψ} ( q ) ) \} \\ = {\int }_{Q} a ( q ; \hat{φ} ( q ) , \hat{ψ} ( q ) ) d π ( q ) \\ = - {〈 𝒜 \hat{φ} , \hat{ψ} 〉}_{{𝒱}^{*} , v} ,\end{matrix}$$

for \hat{φ}, \hat{ψ}∈ [symbol] . As in the deterministic setting, the operator d is regularly dissipative and self-adjoint and can be restricted to Dom ( [symbol] )={φ∈ [symbol] : [symbol] φ∈ [symbol] } as the infinitesimal generator of a uniformly exponentially stable, analytic semigroup { [symbol] (t):t≥0} of bounded, self-adjoint, linear operators on [symbol] .

Define the operators [symbol] ∈ [symbol] ( [symbol] , [symbol] ), [symbol] ₁∈ [symbol] ( [symbol] ², [symbol] ), and [symbol] , [symbol] ∈ [symbol] ( [symbol] , [symbol] ) by [symbol] u= [symbol] _{π}{B(q)}u, u∈ [symbol] , [symbol] ₁ω= [symbol] _{π}{B₁}ω=B₁ω, ω∈ [symbol] ², [symbol] \hat{φ}= [symbol] _{π}{C\hat{φ}}, and [symbol] \hat{φ}= [symbol] _{π}{D\hat{φ}}, \hat{φ}∈ [symbol] , respectively Then set [symbol] = [symbol] (τ)∈ [symbol] ( [symbol] , [symbol] ), \hat{B}= [symbol] ⁻¹( [symbol] − [symbol] ) [symbol] ∈ [symbol] ( [symbol] , [symbol] ), [symbol] ₁= [symbol] ⁻¹( [symbol] − [symbol] ) [symbol] ₁∈ [symbol] ( [symbol] ², [symbol] ), [symbol] = [symbol] ∈ [symbol] ( [symbol] , [symbol] ), and \hat{D}= [symbol] ∈ [symbol] ( [symbol] , [symbol] ), where [symbol] denotes the identity operator on [symbol] .

With these definitions, we then consider the discrete-time linear quadratic regulator problem in [symbol] for the quadratic performance index

$$\hat{𝒥} ( u ) = {\sum }_{k = {k}_{o}}^{{k}_{1} - 1} \{ {〈 \hat{Q} {x}_{k} , {x}_{k} 〉}_{ℋ} + \hat{r} {u}_{k}^{2} \} + {〈 \hat{𝒢} {x}_{{k}_{1}} , {x}_{{k}_{1}} 〉}_{ℋ}$$

subject to the discrete-time linear system

xₖ₊₁= [symbol] xₖ+ [symbol] uₖ+ [symbol] ₁ω(kτ), x₀=\hat{φ}₀,

yₖ= [symbol] xₖ+ζ(kτ), k=k₀, k₀+1, k₀+2, . . . ,

where \hat{Q}, [symbol] ∈ [symbol] ( [symbol] , [symbol] ) are given by \hat{Q}= [symbol] , and [symbol] =ρ [symbol] , respectively and \hat{φ}₀∈ [symbol] . Note that in light of our definitions, the quadratic performance index is the same.

In what follows we will only concern ourselves with the infinite horizon problem (i.e. when k₁=∞ and ρ=0); the results for the finite horizon problem are analogous. The uniform exponential stability of the semigroup { [symbol] (t):t≥0} and therefore of [symbol] as well guarantee that there exists a unique solution, and consequently that an admissible control exists for any initial value. Moreover, we have for any admissible control, lim_{k→∞}∥xₖ [symbol] =0. It follows that there exists a unique positive semi-definite self-adjoint solution to the ARE

\hat{Π}= [symbol] *[\hat{Π}−\hat{Π} [symbol] ( [symbol] + [symbol] *\hat{Π} [symbol] )⁻¹ [symbol] *\hat{Π}] [symbol] +

[symbol]

the optimal input in closed-loop linear state feedback form is given by:

ūₖ=− [symbol] x̅ₖ=− [symbol] [symbol] , x̅ₖ [symbol] , k=k₀, k₀+1, . . . ,

where

[symbol]

={\hat{r}+ [symbol] *\hat{Π} [symbol] }⁻¹ [symbol] *\hat{Π} [symbol] ,

\hat{ƒ}= [symbol] * is the corresponding functional gain, [symbol] (ū)= [symbol] \hat{Π}\hat{φ}₀, \hat{φ}₀ [symbol] , and that the optimal trajectory {x̅ₖ}ₖ₌ₖ_{₀}^{∞} is given by x̅ₖ₊₁=( [symbol] − [symbol] [symbol] )x̅ₖ, x₀=\hat{φ}₀.

To construct the compensator, the observer takes the form

\tilde{x}ₖ₊₁= [symbol] \tilde{x}ₖ+ [symbol] uₖ+ [symbol] (y(kτ)− [symbol] \tilde{x}ₖ), \tilde{x}ₖ_{₀}=\tilde{φ}₀,

k≥ k₀, where \tilde{φ}₀∈ [symbol] is arbitrary and the operator observer gain [symbol] ∈ [symbol] ( [symbol] , [symbol] ) is given by:

[symbol]

= [symbol] \tilde{Π} [symbol] *{σ²+ [symbol] \tilde{Π} [symbol] *}⁻¹

with the operator \tilde{Π} the unique positive semi-definite self-adjoint solution guaranteed to exist to the ARE given by:

\tilde{Π}= [symbol] [\tilde{Π}−\tilde{Π} [symbol] *(σ²+ [symbol] \tilde{Π} [symbol] *)⁻¹ [symbol] \tilde{Π}] [symbol] *+

[symbol]

where \tilde{Q} [symbol] ₁Σ [symbol] *₁∈ [symbol] ( [symbol] , [symbol] ). The optimal LQG compensator is then given by ũₖ=− [symbol] \tilde{x}ₖ=− [symbol] \hat{ƒ}, \tilde{x}ₖ [symbol] [symbol] , k=k₀, k₀+1, k₀+2, . . . , where the feedback operator [symbol] and functional control gains \hat{ƒ} are given by: 31 and 30, respectively. Note that since [symbol] ∈ [symbol] ( [symbol] , [symbol] ), it follows that in fact [symbol] =\tilde{ }∈ [symbol] . The element [symbol] in [symbol] is the optimal functional observer gain. Finally, we note that the spectrum for the closed-loop compensator system is given by:

$$σ ( 𝒮 ) = σ ( [ \begin{matrix}\hat{𝒜} & - \hat{ℬ} \hat{ℱ} \\ \tilde{ℒ} \hat{𝒞} & \hat{𝒜} - \hat{ℬ} \hat{ℱ} - \hat{ℒ} \hat{𝒞}\end{matrix} ] )$$

from which it is not difficult to argue that in fact σ( [symbol] )=σ( [symbol] )− [symbol] )∪σ( [symbol] − [symbol] ).

Approximation and Convergence (Section 14. B). The theory presented in Sections above tells us how to proceed here. We need only describe (1) how to construct a sequence of finite-dimensional approximating subspaces of [symbol] ^{N}, whose corresponding sequence of orthogonal projections converges strongly to the identity in [symbol] , and (2) how to define appropriately converging sequences of approximating operators to [symbol] , and \tilde{Q}.

Let N represent the multi-index N=(n, m₁, m₂, . . . , m₆) and we write N→∞ we mean n→∞ and mᵢ→∞, i=1,2, . . . . 6. We assume that the random parameter qᵢ has support [aᵢ, bᵢ], i=1,2, . . . ,6, all assumed to be bounded. Let Q be the compact subset of [symbol] ⁶ given by Q=xᵢ₌₁⁶[aᵢ, bᵢ]. For i=1,2, . . . , 6, partition [aᵢ, bᵢ] into mᵢ equal subintervals, and let χⱼ^{m}^{ᵢ} denote the characteristic function of the j-th subinterval, j=1,2, . . . , mᵢ. For n=1,2, . . . let {φⱼⁿ}ⱼ₌₀ⁿ denote the standard linear B-splines on [0,1] with respect to the uniform mesh

$$\{ 0 , \frac{1}{n} , \frac{2}{n} , … , 1 \}$$

and set \hat{φ}ⱼⁿ=(φⱼⁿ(0), φⱼⁿ(1), φⱼⁿ)∈V. Let J denote a multi-index of the form J=(j₀, j₁, . . . j₆) where j₀∈{0,1,2, . . . , n} and jᵢ∈, i=1,2, . . . ,6. Then set Φ_{J}^{N}=\hat{φ}ⱼ_{₀}ⁿΠᵢ₌₁⁶χⱼ_{ᵢ}^{m}^{ᵢ}and let [symbol] ^{N}=span_{J}{Φ_{J}^{N}}, let [symbol] ^{N}: [symbol] → [symbol] ^{N} denote the orthogonal projection of [symbol] onto [symbol] ^{N}. Standard arguments from the theory of splines and piecewise constant approximation in L² can be used to argue that [symbol] ^{N} converges strongly to the identity in both [symbol] and [symbol] .

Since [symbol] ^{N} is a subspace of [symbol] (and [symbol] ) we simply set [symbol] ^{N}= [symbol] [symbol] ^{N} for all multi-indices N. We obtain [symbol] ^{N}∈ [symbol] ( [symbol] ^{N}, [symbol] ^{N}) via a Galerkin approach as described above and set [symbol] ^{N}=exp( [symbol] ^{N}τ)∈ [symbol] ( [symbol] ^{N}, [symbol] ^{N}). We then set [symbol] ^{N}=( [symbol] ^{N})⁻¹( [symbol] ^{N}− [symbol] ^{N}) [symbol] ^{N} [symbol] ∈ [symbol] ( [symbol] , [symbol] ^{N}). Similarly we set [symbol] ₁^{N}=( [symbol] ^{N})⁻¹( [symbol] ^{N}− [symbol] ^{N}) [symbol] ^{N} [symbol] ₁∈ [symbol] ( [symbol] ², [symbol] ^{N}), and then set \tilde{Q}^{N}= [symbol] ₁^{N}Σ( [symbol] ₁^{N})*∈ [symbol] ( [symbol] ^{N}, [symbol] ^{N}). We note also that \hat{Q}^{N}= [symbol] ^{N}\hat{Q} [symbol] ^{N}=( [symbol] ^{N})* [symbol] ^{N}, where \hat{D}^{N}= [symbol] [symbol] ^{N}. Arguments from functional analysis (specifically, linear semigroup theory) can then be used to argue the requisite convergence.

We then consider the sequence of finite-dimensional approximating LQR/LQG compensator problems on the infinite-time horizon to minimize

$${\hat{𝒥}}^{N} ( {u}^{N} ) = {\sum }_{k = {k}_{0}}^{∞} {〈 {\hat{Q}}^{N} {x}_{k}^{N} , {x}_{k}^{N} 〉}_{ℋ} + {\hat{r} ( {u}_{k}^{N} )}^{2}$$

subject to

xₖ₊₁^{N}= [symbol] ^{N}xₖ^{N}+ [symbol] ^{N}uₖ^{N}+ [symbol] ₁^{N}ω(kτ), x₀^{N}= [symbol] ^{N}\hat{φ}₀.

yₖ^{N}= [symbol] ^{N}xₖ^{N}+ζ(kτ), k=k₀k₀+1, k₀+2, . . .

As in the infinite-dimensional case, the unique solution to this problem is given in closed-loop linear state feedback form by [10] ūₖ^{N}= [symbol] ^{N}x̅ₖ=− [symbol] \hat{ƒ}^{N}, x̅ₖ^{N} [symbol] , k=k₀, k₀+1, . . . , where

[symbol]

^{N}={\hat{r}+( [symbol] ^{N})*\hat{Π}^{N}) [symbol] ^{N}}⁻¹( [symbol] ^{N})*\hat{Π}^{N} [symbol] ^{N}

and \hat{Π}^{N} is the unique positive semi-definite, symmetric solution to the approximating ARE,

\hat{Π}^{N}=( [symbol] ^{N})*[\hat{Π}^{N}−\hat{Π}^{N} [symbol] ^{N}(\hat{r}+( [symbol] ^{N})*\hat{Π}^{N} [symbol] ^{N})⁻¹( [symbol] ^{N})*\hat{Π}^{N}] [symbol] ^{N}+( [symbol] ^{N})* [symbol] ^{N}

where x̅ₖ^{N} denotes the trajectory given by 32 with uₖ^{N}=ūₖ^{N}, k=k₀, k₀+1, . . . and \hat{ƒ}^{N} denotes the optimal functional control gains. It follows that [symbol] ^{N}(ū^{N})= [symbol] \hat{Π}^{N} [symbol] ^{N}\hat{φ}₀, [symbol] ^{N}\hat{φ}₀ [symbol] [symbol] and that the optimal trajectory is given by x̅ₖ₊₁^{N}=( [symbol] ^{N}− [symbol] ^{N} [symbol] ^{N})x̅ₖ^{N}, x₀^{N}= [symbol] ^{N}\hat{φ}₀. We note that in actual practice, the control applied would be ūₖ^{N}=− [symbol] \hat{ƒ}^{N}, x̅ₖ [symbol] [symbol] , k=k₀k₀+1, . . . , where x̅ₖ denotes the trajectory given with uₖ=ūₖ^{N}, k=k₀, k₀+1, . . . The approximating observer is given by:

\tilde{x}ₖ₊₁^{N}= [symbol] ^{N}\tilde{x}ₖ^{N}+ [symbol] ^{N}uₖ^{N}+ [symbol] ^{N}(y(kτ)− [symbol] ^{N}\tilde{x}ₖ^{N})

\tilde{x}₀^{N}= [symbol] ^{N}\tilde{φ}₀

where [symbol] ^{N}∈ [symbol] ( [symbol] , [symbol] ^{N}) is given by:

[symbol]

^{N}= [symbol] ^{N}\tilde{Π}^{N} [symbol] *{σ²+ [symbol] ^{N}\tilde{Π}^{N}( [symbol] ^{N})*}⁻¹

with Π^{N} the unique positive semi-definite symmetric solution to the

\tilde{Π}^{N}= [symbol] ^{N}[\tilde{Π}^{N}−\tilde{Π}^{N}( [symbol] ^{N})*(σ²+ [symbol] ^{N}\tilde{Π}^{N}( [symbol] ^{N})*)⁻¹ [symbol] ^{N}\tilde{Π}^{N}]( [symbol] ^{N})*+ [symbol] ₁^{N}Σ( [symbol] ₁^{N})*.

The equations are operator equations, albeit finite dimensional ones. In order to actually carry out computations (i.e. by using standard ARE solvers) these equations must be converted to equivalent matrix equations. Since the basis we have chosen for W^{N} is not orthonormal, some care must be exercised in making this conversion so as to obtain a standard symmetric matrix ARE.

The approximating compensator is then given by

$$\begin{matrix}{\overline{u}}_{k}^{N} = {\hat{ℱ}}^{N} {\tilde{x}}_{k}^{N} = - {〈 {\hat{f}}^{N} , {\tilde{x}}_{k}^{N} 〉}_{ℋ} \\ = - {\int }_{Q} \{ \begin{matrix}\frac{{q}_{1}}{{q}_{3}} {\hat{f}}_{3}^{N} ( 0 , q ) {\tilde{x}}_{3 , k}^{N} ( 0 , q ) + \frac{{q}_{1}}{{q}_{3}} {\hat{f}}_{3}^{N} ( 1 , q ) {\tilde{x}}_{3 , k}^{N} ( 1 , q ) + \\ {\int }_{0}^{1} {\hat{f}}_{3}^{N} ( η , q ) {\tilde{x}}_{2 , k}^{N} ( η , q ) d η\end{matrix} \} \\ d π ( q ) , k = {k}_{0} , {k}_{0} + 1 , … ,\end{matrix}$$

where in the above expression we have used the following notational convention \hat{ƒ}^{N}=(\hat{ƒ}₁^{N}, \hat{ƒ}₂^{N}, \hat{ƒ}₃^{N})∈ [symbol] ^{N} and \tilde{x}ₖ^{N}=(\tilde{x}_{1,k}^{N}, \tilde{x}_{2,k}^{N}, \tilde{x}_{3,k}^{N})∈ [symbol] ^{N}. We note that because the generator [symbol] ^{N} of the approximating semigroup {exp( [symbol] ^{N}t):t≥0}, was constructed using a Galerkin approach, we are guaranteed the existence of unique positive semi-definite symmetric solutions to the AREs for the same reasons that this is true in the infinite-dimensional case stated in the previous sub-section. In addition, the convergence results given herein apply and finally we note that approximating closed loop eigenvalues can be obtained as

$$\begin{matrix}σ ( {δ}^{N} ) = σ ( [ \begin{matrix}{\hat{𝒜}}^{N} & - {\hat{ℬ}}^{N} {\hat{ℱ}}^{N} \\ {\tilde{ℒ}}^{N} {\hat{𝒞}}^{N} & {\hat{𝒜}}^{N} - {\hat{ℬ}}^{N} {\hat{ℱ}}^{N} - {\tilde{ℒ}}^{N} {\hat{𝒞}}^{N}\end{matrix} ] ) \\ = σ ( {\hat{𝒜}}^{N} - {\hat{ℬ}}^{N} {\hat{ℋ}}^{N} ) ⋃ σ ( {\hat{𝒜}}^{N} - {\tilde{ℒ}}^{N} {\hat{𝒞}}^{N} )\end{matrix}$$

Numerical Results (Section 15). We consider a system of the general form of the one given previously. In particular we let q₁=0.2, q₂=0.5, q₃=0.5, q₄=0.5, q₅=0.5, q₆=0.5, σ₁=0.05, σ₂=0.05, σ=0.05 k₀=0, k₁=∞, ρ=0, and \hat{r}=0.1. We assume further that we do not actually know the precise value of q₁, but rather only that it is random with q₁˜Beta (α, β) with α=3 and β=2. We take the sampling interval to be τ=0.1 and the discretization level of η∈[0,1] and q₁∈[0,1] to be given by the multi-index N=(n, m).

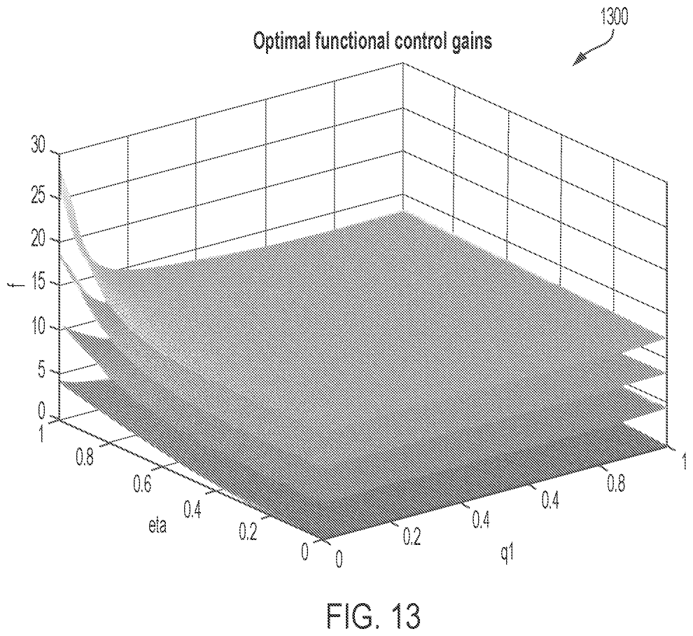

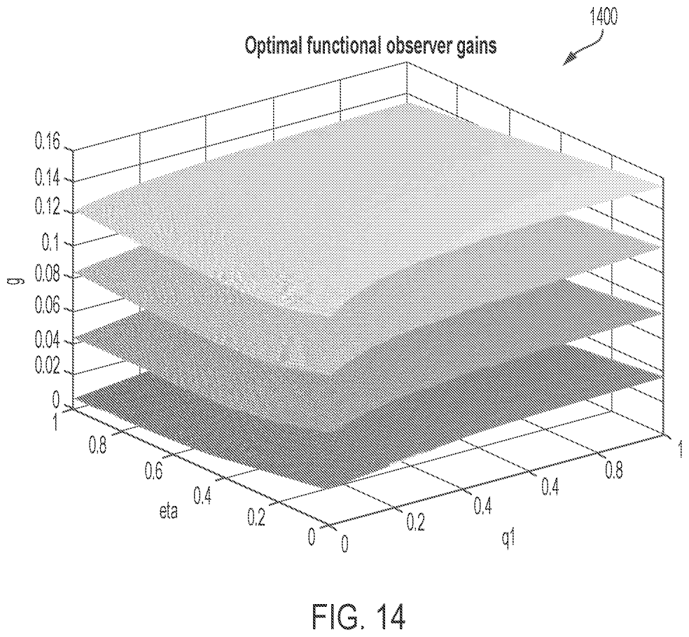

In FIG. 13 and FIG. 14 we plot the functional control and observer gains for (from lower to upper) n=m=4,8,16, and 32. FIG. 13 depicts functional control 1300 gains and FIG. 14 depicts observer gains 1400. The plots have been off-set so that they can be distinguished from one another. Table I contains the L² norm of the difference between the approximating optimal functional control gains and the infinite-dimensional (computed with n=m=32) control gains. Tables II contains the L² norm of the difference between the approximating optimal functional control gains and the infinite-dimensional (computed with n=m=32) observer gains.

TABLE I

| m = n | 4 | 8 | 12 | 16 | 20 | 24 | 28 |
|---|---|---|---|---|---|---|---|
| Norm | 18.00 | 10.00 | 5.18 | 2.61 | 1.25 | 0.54 | 0.17 |
| (×10⁻⁴) | | | | | | | |

TABLE II

| m = n | 4 | 8 | 12 | 16 | 20 | 24 | 28 |
|---|---|---|---|---|---|---|---|
| Norm | 12.62 | 7.39 | 4.81 | 3.21 | 2.09 | 1.24 | 0.56 |
| (×10⁻⁴) | | | | | | | |

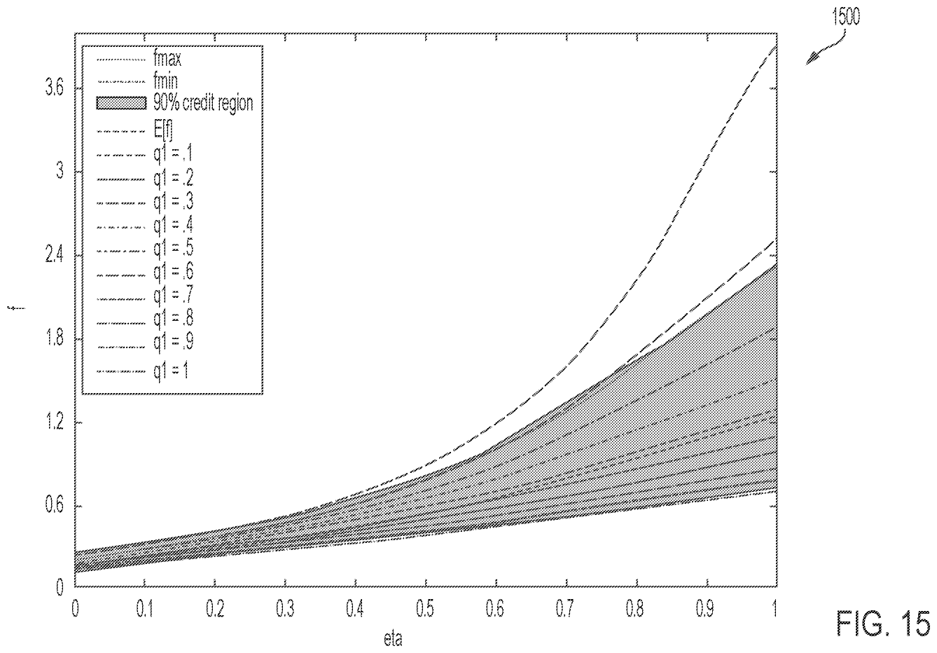

In Table III we show the optimal functional control gains \hat{ƒ}₁ and \hat{ƒ}₂ and iwe have plotted the optimal functional control gains \hat{ƒ}₃ for the full state feedback controller when q₁=0.1j,j=1,2, . . . ,9,10 all computed with n=32. In the same table and figure we have also tabulated and plotted the expected value of the optimal functional control gains, [symbol] _{π}[\hat{ƒ}] computed using our approach with n=m=16 and q₁˜Beta (α, β) with α=3 and β=2. In addition, since our scheme yields the approximating optimal control (and observer) gains as a function of q₁, we can readily compute 90% credible intervals and bands for the optimal control gains computed with our method. In FIG. 15, chart 1500 has a shaded region and the shaded region is the 90% credible band centered at the mean for the optimal functional control gains \hat{ƒ}₃ computed using our method.

In Table IV we show the values of the performance index, J(u), when the system was simulated with different approximating optimal controllers/compensators. We took x(0, η)=1.0,0≤η≤1, w(0)=1.0, v(0)=1.0 and computed the approximating controllers with either n=32 or n=m=32. We took the plant parameter values to be q=[0.2,0.5,0.5,0.5.0.5,0.5]^{T}, the final time to be T=10.0, and the length of the sampling interval to be τ=0.1. The standard deviations of the noise processes were taken to be σ₁=σ₂=σ=0.05 and the control penalty weight was r\hat{ }=0.1. We set the seed in Matlab's random number generator to be equal to one in all of the simulations. We simulated the linearized plant using our spline model with n=64 and for our scheme we assumed that q₁=q₁ was random with q₁˜Beta(3, 2).

TABLE III

| q₁ | 0.1 | 0.3 | 0.5 | 0.7 | 0.9 | Eπ[q₁] |
|---|---|---|---|---|---|---|
| f₁ | 0.2630 | 0.2128 | 0.1740 | 0.1462 | 0.1258 | 0.1603 |
| f₂ | 3.9239 | 1.8792 | 1.2699 | 0.9654 | 0.7807 | 1.2339 |

TABLE IV

| Con/Comp | 1 | 2 | 3 | 6 |
|---|---|---|---|---|
| J(u) | 9.07 | 5.08 | 7.78 | 5.60 |

TABLE V

| Con/Comp | 1 | 2 | 3 | 4 | 5 | 6 |
|---|---|---|---|---|---|---|
| J(u) | 21.99 | 10.88 | 10.89 | 11.92 | 11.70 | 11.57 |

In Table IV Controller/Compensator 1 was no control (i.e. uₖ=0, k=0,1,2, . . . ,99), Controller/Compensator 2 was the optimal infinite-dimensional (n=64) full state feedback controller computed with the plant's value for q₁ to be q₁=0.2, Controller/Compensator 3 was the optimal finite-dimensional (n=32) output feedback compensator computed with the plant's value for q₁ to be the plant value of q₁, q₁=0.2. Controller/Compensator 4 was the optimal finite-dimensional (n=32) output feedback compensator but computed with the incorrect value for q₁, q₁=0.8, Controller/Compensator 5 was the optimal finite-dimensional (n=32) output feedback compensator but computed with q₁= [symbol] [q₁]=0.6, and finally Controller/Compensator 6 was the optimal finite-dimensional (n=32, m=32) output feedback compensator computed using the approach we developed.

Finally, in Table V we show results of simulating controller/compensator 1, 2, and 3 along with compensator 6, the one developed here, for the case where q₁=q_{1,k}˜Beta (α, β) with α=3 and β=2, k=0,1,2, . . .

We have demonstrated the optimality and convergence of approximating finite-dimensional compensators for a plant of the form herein. As can be seen from the numerical studies in the previous section, we have also demonstrated that our finite-dimensional compensators perform well in both the case where the plant system parameters are fixed but unknown (with known distribution) and where they take on a different random value in each sampling interval. However, the rigorous analysis of the performance of the actual closed loop system (e.g. could the finite-dimensional compensator destabilize the infinite dimensional plant or is the compensator in any sense optimal, etc.) in each of these cases, at present, remains open.

An extension of our results for the LQG compensator problem for random parabolic systems developed here may be contemplated to the LQG tracking problem for random parabolic systems. As was the case with the results presented here, this effort is again motivated by problems involving transdermal alcohol transport and sensing. Specifically, there are two problems of particular interest to us; one is a control problem and the other is an estimation or filtering problem. The first problem is the natural extension of the results presented here for the control of the alcohol clamping studies to experiments whose aim is to have the subject's BAC track or follow a pre-specified trajectory. Once again sensing would be based on observations of transdermal alcohol level. As in the case of the clamping studies, the resulting control problem is complicated by the fact that the underlying model is population-based with only the distribution of the model parameters known.

The second problem of interest to us involves the estimation of BAC or BrAC from TAC measurements. The technology to measure TAC is relatively new. Consequently, researchers and clinicians working in the area of alcohol use disorders have traditionally based their studies and diagnoses almost exclusively on observations of BAC or BrAC. In addition, BAC and BrAC are the preferred measure of intoxication in the consumer (i.e. wearable technology) and forensic (e.g. DUI) communities. Observations of BAC and BrAC are difficult or impossible to collect in a naturalistic setting in the field, while through the use of this new technology, TAC can be. Thus, a reliable means to convert TAC into equivalent BAC/BrAC is desired. The approach we are looking at is to formulate the TAC to BAC/BrAC conversion as an LQG tracking problem wherein the input (i.e. the BAC or BrAC) that forces the model (rather than the plant!) to track the biosensor measured TAC is determined. The underlying diffusion and transport model is augmented with actuator dynamics so that the input penalty term in the quadratic performance index can serve as regularization to mitigate over-fitting. Again, the underlying dynamics are in the form of a population model with only the distributions rather than the actual values of the parameters known.

What is claimed is:

1. A method for converting transdermal alcohol concentration (TAC) to blood or breath alcohol concentration (BAC/BrAC), the method comprising:
measuring, using a biosensor, the TAC of a human;
receiving, by a processor, data corresponding to one or more drinking curves for a population of humans;
receiving, by the processor, data corresponding to at least one of (i) static characteristics of the human, (ii) physiological characteristics of the human, and (iii) current environmental conditions; and
converting, using the processor, the TAC to BAC/BrAC using the data from one or more drinking curves, and the at least one of (i) the static characteristics of the human, (ii) the physiological characteristics of the human, and (iii) the current environmental conditions.

2. The method of claim 1, wherein the data corresponding to the one or more drinking curves includes a measurement of TAC and a measurement of at least one of BAC and BrAC.

3. The method of claim 1, wherein the data corresponding to the one or more drinking curves includes a time sequence of measurements of TAC and a time sequence of measurements of BAC or BrAC, and wherein the method is performed in real time.

4. The method of claim 1, wherein the data corresponding to the static characteristics includes a measurement of at least one of age, sex, ethnicity, height, weight, body fat and muscle, skin color, skin thickness, and skin tortuosity,
wherein the data corresponding to the physiological characteristics includes a measurement of at least one of sweat, skin conductance, skin hydration, exercise, heart rate, blood pressure, blood flow, and stomach content, and
wherein the data corresponding to the current environmental conditions includes a measurement of at least one of ambient temperature, humidity, pressure, GPS, weather, and climate.

5. The method of claim 1, wherein the converting is performed using a deterministic or stochastic finite dimensional autoregressive moving average with exogenous input (ARMAX) input/output model.

6. The method of claim 1, wherein the converting is performed using a blind or Bayesian deconvolution scheme.

7. The method of claim 1, wherein the converting is performed using a lattice filter-based recursive identification scheme.

8. The method of claim 1, wherein the converting is performed using an artificial neural network (ANN) by the processor, wherein the processor is remote from the biosensor and connected to the biosensor by a network.

9. The system of claim 1, wherein the converting is performed using a hidden Markov model (HMM) or a physics-informed hidden Markov model (PIHMM) by the processor.

10. The system of claim 1, wherein the converting is performed using a deconvolution filter based on output feedback linear quadratic Gaussian tracking gain computed by the processor.

11. The system of claim 1, wherein the converting is performed using first principles physics-based forward model with random parameters having distributions fit to population BrAC/TAC data and wherein the fitting the distributions is based on a naïve pooled or mixed effects statistical model using either maximum likelihood, method of moments, or Bayesian techniques by the processor.

12. A system for converting transdermal alcohol concentration (TAC) to blood or breath alcohol concentration (BAC/BrAC), wherein the converting is in real-time with progressive forecasting and modeling techniques and recursive updating methods, the system comprising:
a biosensor for measuring the TAC of a human; and
a processor configured to:
receive data from one or more drinking curves from a population of humans;
receive data corresponding to at least one of (i) static characteristics of the human, (ii) physiological characteristics of the human, and (iii) the current environmental conditions; and
convert, by the processor, in real-time the TAC to BAC/BrAC using the data from one or more drinking curves and the at least one of (i) the static characteristics of the human, (ii) the physiological characteristics of the human, and (iii) the current environmental conditions.

13. The system of claim 10, wherein the processor is remote from the biosensor and is connected to the biosensor via a network.

14. The system of claim 10, further comprising a remote database containing the one or more drinking curves from the population of humans connected to the processor via a network.

15. The system of claim 10, wherein the system comprises a plurality of further biosensors connected to the processor via a network, wherein the processor coverts, in real-time the TAC to BAC/BrAC for each of the plurality of further biosensors.

16. The system of claim 10, wherein the data corresponding to the one or more drinking curves includes a measurement of TAC and a measurement of at least one of BAC and BrAC.

17. The system of claim 10, wherein the data corresponding to the static characteristics includes a measurement of at least one of age, sex, ethnicity, height, weight, body fat and muscle, skin color, thickness, and tortuosity,
wherein the data corresponding to the physiological characteristics includes a measurement of at least one of sweat, skin conductance, skin hydration, exercise, heart rate, blood pressure, blood flow, and stomach content, and
wherein the data corresponding to the current environmental conditions includes a measurement of at least one of ambient temperature, humidity, pressure, GPS location data, weather, and climate.

18. The system of claim 10, wherein the converting is performed in real-time using a deterministic or stochastic finite dimensional autoregressive moving average with exogenous input (ARMAX) input/output model.

19. The system of claim 10, wherein the converting is performed using an artificial neural network (ANN) or a physics-informed neural network (PINN) by the processor.

20. A biosensor device for converting transdermal alcohol concentration (TAC) to blood or breath alcohol concentration (BAC/BrAC), the device comprising:
a wearable sensor contactable to a human skin to measure the TAC of the human;
a processor connected to the wearable sensor and connectable to a network, the processor configured to receive, via the network, data corresponding to one or more drinking curves for a population of humans;
the processor configured to convert TAC to BAC/BrAC using (i) the data from one or more drinking curves and (ii) the measured TAC.
